(19) United States
(12) Patent Application Publication
(10) Pub. No.: US 2008/0254059 A1
(43) Pub. Date: Oct. 16, 2008

(54) Adenovirus Serotype 26 Vectors, Nucleic Acid and Viruses Produced Thereby

(76) Inventors: Andrew J. Bett, Lansdale, PA (US); Danilo R. Casimiro, Harleysville, PA (US); John W. Shiver, Doylestown, PA (US); Emilio A. Emini, Wayne, PA (US); Michael Chastain, Seattle, WA (US); David C. Kaslow, Wayne, PA (US)

Correspondence Address: MERCK AND CO., INC, P O BOX 2000, RAHWAY, NJ, 07065-0907, US

(21) Appl. No.: 11/884,086

(22) PCT Filed: Feb. 7, 2006
(86) PCT No.: PCT/US2006/004060

§ 371 Date: Aug. 8, 2007

Related U.S. Application Data
(60) Provisional application No. 60/652,041, filed on Feb. 11, 2005.

(51) Int. Cl. A61K 39/00; C12N 15/00; C12N 5/06; C12N 15/87; A61P 37/00; C12N 7/00; A61K 35/76; A61K 31/70

(52) U.S. Cl. 424/199.1; 435/320.1; 435/325; 435/455; 435/235.1; 424/93.2; 514/44

(57) ABSTRACT

Adenoviral serotypes differ in their natural tropism. The various serotypes of adenovirus have been found to differ in at least their capsid proteins (e.g., penton-base and hexon proteins), proteins responsible for cell binding (e.g., fiber proteins), and proteins involved in adenovirus replication. This difference in tropism and capsid proteins among serotypes has led to many research efforts aimed at redirecting the adenovirus tropism by modification of the capsid proteins. The present invention bypasses such requirement for capsid protein modification as it presents a recombinant, replication-defective adenovirus of serotype 26, a rare adenoviral serotype, and methods for generating the alternative, recombinant adenovirus. Additionally, means of employing the recombinant adenovirus for delivery and expression of heterologous genes are provided.

### CROSS-REFERENCE TO RELATED APPLICATIONS

This application claims the benefits of U.S. provisional application Ser. No. 60/652,041, filed Feb. 11, 2005.

### BACKGROUND OF THE INVENTION

Adenoviruses are nonenveloped, icosahedral viruses that have been identified in several avian and mammalian hosts; Home et al., 1959 J. Mol. Biol. 1:84-86; Horwitz, 1990 In Virology, eds. B. N. Fields and D. M. Knipe, pps. 1679-1721. The first human adenoviruses (Ads) were isolated over four decades ago. Since then, over 100 distinct adenoviral serotypes have been isolated which infect various mammalian species, 51 of which are of human origin; Straus, 1984, In The Adenoviruses, ed. H. Ginsberg, pps. 451-498, New York: Plenus Press; Hierholzer et al., 1988 J. Infect. Dis. 158:804-813; Schnurr and Dondero, 1993, Intervirology; 36:79-83; De Jong et al., 1999 J. Clin. Microbiol., 37:3940-5. The human serotypes have been categorized into six subgenera (A-F) based on a number of biological, chemical, immunological and structural criteria which include hemagglutination properties of rat and rhesus monkey erythrocytes, DNA homology, restriction enzyme cleavage patterns, percentage G+C content and oncogenicity; Straus, supra; Horwitz, supra.

Adenoviruses are attractive targets for the delivery and expression of heterologous genes. Adenoviruses are able to infect a wide variety of cells (dividing and non-dividing), and are very efficient in introducing their DNA into infected host cells. Adenoviruses have not been found to be associated with severe human pathology in immuno-competent individuals. The viruses can be produced at high virus titers in large quantities. The adenovirus genome is very well characterized, consisting of a linear double-stranded DNA molecule of approximately 30,000-45,000 base pairs. Furthermore, despite the existence of several distinct serotypes, there is some general conservation found amongst the various serotypes.

Safety in utilizing adenoviruses as gene delivery vehicles can be enhanced by rendering the viruses replication-defective through deletion/modification of the essential early-region 1 (“E1”) of the viral genomes, rendering the viruses devoid (or essentially devoid) of E1 activity and, thus, incapable of replication in the intended host/vaccinee; see, e.g., Brody et al, 1994 Ann NY Acad Sci., 716:90-101. Deletion of adenoviral genes other than E1 (e.g., in E2, E3, and/or E4), furthermore, creates adenoviral vectors with greater capacity for heterologous gene inclusion. Presently, two well-characterized adenovirus serotypes of subgroup C, serotypes 5 (“Ad5”) and 2 (“Ad2”) form the basis of the most widely used gene delivery vectors.

One concern surrounding the use of adenovectors relates to the potential for cellular and humoral immune responses elicited by and directed towards the viruses themselves (Chirmule et al., 1999 Gene Ther. 6:1574-1583). Although an immune response associated with the initial administration of a vector may be advantageous (Zhang et al., 2001 Mol. Ther. 3:697-707), the generation of systemic levels of adenovirus-specific neutralizing antibodies may cause poor transduction when the vectors are readministered (booster immunizations; Kass-Eisler et al., 1996 Gene Ther. 3:154-162; Chirmule et al., 1999 J. Immunol. 163:448-455). The scientific literature and data from our own epidemiological studies suggest that most North Americans have anti-Ad5 neutralizing antibody titers, and about one third have relatively high titers (>200). Other parts of the world typically exhibit higher frequencies and levels of anti-Ad5 antibodies. Serospecific antibodies to these and other adenoviral serotypes resulting from such natural adenovirus infections in humans may affect the extent of response to the administration of heterologous polypeptides by adenovectors; Chirmule et al., 1999 Gene Ther. 6:1574-1583. Accordingly, there is a need to develop adenoviral vectors based on alternate adenovirus serotypes as gene transfer vectors, particularly where the serotypes are less prevalent than adenovirus serotypes 2 and 5.

Adenovirus serotype 26, a subgroup D adenovirus, was originally isolated in 1961 and established as a recognized reference strain in 1963 (L. Rosen et al., 1961 J. Proc. Soc. Exp. Biol. Med. 107:434-437; H. G. Pereira et al., 1963 Virology 20:613-620). Its antigenic relationship to 46 other human adenoviruses determined in reference horse antisera has been discussed; J. C. Hierholzer et al., 1991 Arch. Virol. 121:179-197. There is some sequence information published for Ad26. Partial sequences for Ad26 hexon protein (1129 and 916 bps) were disclosed in Takeuchi et al., 1999 J. Clin. Microbiol. 37:3392-3394 (GenBank Accession No. AB023554); and Shimada et al., 2004 J. Clin. Microbiol. 42:1577-1584 (GenBank Accession No. AB099360). Sequence for the virus associated RNA region for Ad26 and partial sequence for the pre-terminal protein and 52/55K proteins (521 bp) was disclosed in Ma & Matthews, 1996 J. Virol., 70:5083-5099, and GenBank (Accession No. U52546).

The fields of vaccines and gene therapy would greatly benefit from additional knowledge concerning alternative adenoviral serotypes, particularly those serotypes such as Ad26 which are not well represented in the human population. Of particular interest are recombinant adenoviral vectors based on alternative adenoviral serotypes, and means of obtaining such recombinant adenoviral vectors. This need in the art is met with the disclosure of the present application related to recombinant adenoviral vectors based on adenoviral serotype 26.

### SUMMARY OF THE INVENTION

The present invention relates to recombinant, replication-defective adenovirus vectors of serotype 26, a rare adenoviral serotype, and methods for generating recombinant adenoviral vectors based on the alternative serotype. Additionally, means of employing said recombinant adenoviral vectors in the delivery and expression of heterologous genes are provided. Recombinant, replication-defective adenoviral vectors of serotype 26 wherein the vectors comprise one or more transgenes (heterologous genes) operatively linked to regulatory sequences are, furthermore, disclosed herein. Host administration of such recombinant adenovirus serotype 26 vectors, whether administered alone or in a combined modality and/or prime boost regimen, results in the efficient expression of the incorporated transgene and effectively induces an immune response capable of specifically recognizing the particular antigen administered. Recombinant viruses in accordance with this description have an innate ability to evade pre-existing immunity directed towards adenovirus serotypes which tend to be more prevalent in the human population (e.g., Ad5 and Ad2). Use of such recombinant adenoviruses, therefore, offers an enhanced means for expressing a particular heterologous nucleic acid of interest.

### BRIEF DESCRIPTION OF THE DRAWINGS

FIGS. 1A-1 through 1A-11 illustrate a nucleic acid sequence for adenovirus serotype 26 (SEQ ID NO: 1). Available ATCC product numbers for Ad26 are as follows: VR-1104, VR-1104AS/RB, VR-1104PI/RB, and VR-224.

FIG. 2 illustrates a homologous recombination scheme utilized to recover pAd26ΔE1ΔE4Ad5Orf6.

FIGS. 3A-1 through 3A-9 illustrate a nucleic acid sequence for pAd26ΔE1ΔE4Ad5Orf6 (SEQ ID NO: 2).

FIG. 4 illustrates a homologous recombination scheme utilized to recover pAd26ΔE1ΔE3ΔE4Ad5Orf6.

FIG. 5 illustrates, in tabular format, levels of Gag-specific T cell responses induced in macaques using Ad26ΔE1gagΔE4Ad5Orf6 and MRKAd5gag vectors in a prime-boost vaccination protocol. Values reflect the mock-subtracted numbers of IFN-γ secreting cells per million PBMC; wk, week.

FIG. 6 illustrates, in tabular format, the numbers of CD4+ and CD8+Gag-specific T cells per million lymphocytes in Ad26-immunized macaques at week 8.

FIG. 7 illustrates the nucleic acid sequence (SEQ ID NO: 3) of an optimized human HIV-1 gag open reading frame.

FIG. 8 illustrates the nucleic acid sequence encoding the gag expression cassette (SEQ ID NO: 4). The various regions of the figure are as follows: (1) a first underlined segment of nucleic acid sequence encoding the immediate early gene promoter region from human cytomegalovirus; (2) a first segment of lowercase letters which is not underlined, which segment of DNA contains a convenient restriction enzyme site; (3) a region in caps which contains the coding sequence of HIV-1 gag; (4) a second segment of lowercase letters which is not underlined, which segment of DNA contains a convenient restriction enzyme site; and (5) a second underlined segment, this segment containing nucleic acid sequence encoding a bovine growth hormone polyadenylation signal sequence.

FIGS. 9A-1 through 9A-2 illustrate a codon optimized wt-pol sequence, wherein sequences encoding protease (PR) activity are deleted, leaving codon optimized “wild type” sequences which encode RT (reverse transcriptase and RNase H activity) and IN integrase activity (SEQ ID NO: 6). The open reading frame starts at an initiating Met residue at nucleotides 10-12 and ends at a termination codon at nucleotides 2560-2562.

FIGS. 10A-1 through 10A-2 illustrate the open reading frame (SEQ ID NO: 7) of the wild type pol construct disclosed as SEQ ID NO: 6.

FIGS. 11A-1 through 11A-3 illustrate the nucleotide (SEQ ID NO: 8) and amino acid sequence (SEQ ID NO: 9) of IA-Pol. Underlined codons and amino acids denote mutations, as listed in Table 1 herein.

FIG. 12 illustrates a codon optimized version of HIV-1 jrfl nef (SEQ ID NO: 10). The open reading frame starts at an initiating methionine residue at nucleotides 12-14 and ends at a “TAA” stop codon at nucleotides 660-662.

FIG. 13 illustrates the open reading frame (SEQ ID NO: 11) of codon optimized HIV jrfl Nef.

FIGS. 14A-1 through 14A-2 illustrate a nucleotide sequence comparison between wild type nef (jrfl) and codon-optimized nef. The wild type nef gene from the jrfl isolate consists of 648 nucleotides capable of encoding a 216 amino acid polypeptide. WT, wild type sequence (SEQ ID NO: 12); opt, codon-optimized sequence (contained within SEQ ID NO: 10). The Nef amino acid sequence is shown in one-letter code (SEQ ID NO: 11).

FIG. 15 illustrates nucleic acid (herein, “opt nef (G2A, LLAA)”; SEQ ID NO: 13) which encodes optimized HIV-1 Nef wherein the open reading frame encodes for modifications at the amino terminal myristylation site (Gly-2 to Ala-2) and substitution of the Leu-174-Leu-175 dileucine motif to Ala-174-Ala-175. The open reading frame starts at an initiating methionine residue at nucleotides 12-14 and ends at a “TAA” stop codon at nucleotides 660-662.

FIG. 16 illustrates the open reading frame (SEQ ID NO: 14) of opt nef (G2A, LLAA).

FIG. 17 illustrates nucleic acid (herein, “opt nef (G2A)”; SEQ ID NO: 15) which encodes optimized HIV-1 Nef wherein the open reading frame encodes for modifications at the amino terminal myristylation site (Gly-2 to Ala-2). The open reading frame starts at an initiating methionine residue at nucleotides 12-14 and ends at a “TAA” stop codon at nucleotides 660-662.

FIG. 18 illustrates the open reading frame (SEQ ID NO: 16) of opt nef (G2A).

FIG. 19 illustrates a schematic presentation of nef and nef derivatives. Amino acid residues involved in Nef derivatives are presented. Glycine 2 and Leucine 174 and 175 are the sites involved in myristylation and dileucine motif, respectively.

FIG. 20 illustrates the nucleic acid sequence encoding the SEAP expression cassette (SEQ ID NO: 17). The various regions of the figure are as follows: (1) a first underlined segment of nucleic acid sequence encoding the immediate early gene promoter region from human cytomegalovirus; (2) a first segment of lowercase letters which is not underlined, which segment of DNA contains a convenient restriction enzyme site; (3) a region in caps which contains the coding sequence of the human placental SEAP gene; (4) a second segment of lowercase letters which is not underlined, which segment of DNA contains a convenient restriction enzyme site; and (5) a second underlined segment, this segment containing nucleic acid sequence encoding a bovine growth hormone polyadenylation signal sequence.

### DETAILED DESCRIPTION OF THE INVENTION

Rare adenoviral serotypes possess an inherent advantage over the more commonly exploited adenoviral serotypes such as adenoviral serotypes 2 and 5 primarily because preexisting immunity is unlikely to limit their efficient delivery to, and expression of heterologous genes at, their target site. Different adenoviral serotypes also exhibit distinct tropisms by reason of their varying capsid structure and, thus, present the potential for targeting different tissues and possibly leading to the elicitation of superior immune responses when used for vaccine or gene therapy purposes. These rare serotypes when rendered replication-defective, however, have typically been difficult to propagate and rescue and, therefore, have not been fully characterized to date.

Applicants have recently managed to successfully rescue and propagate one such rare, replication-defective alternative serotype, adenovirus serotype 26, a subgroup D adenovirus, and herein demonstrate the effective functioning of these adenoviruses in the delivery and expression of heterologous transgenes.

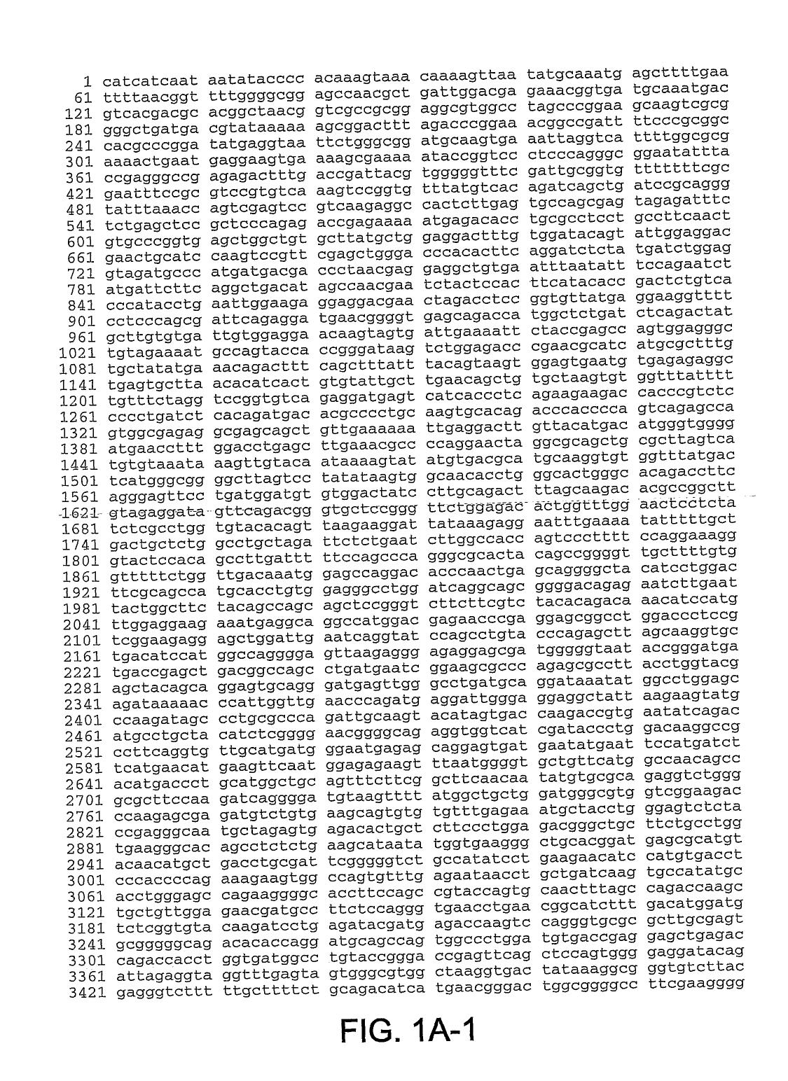

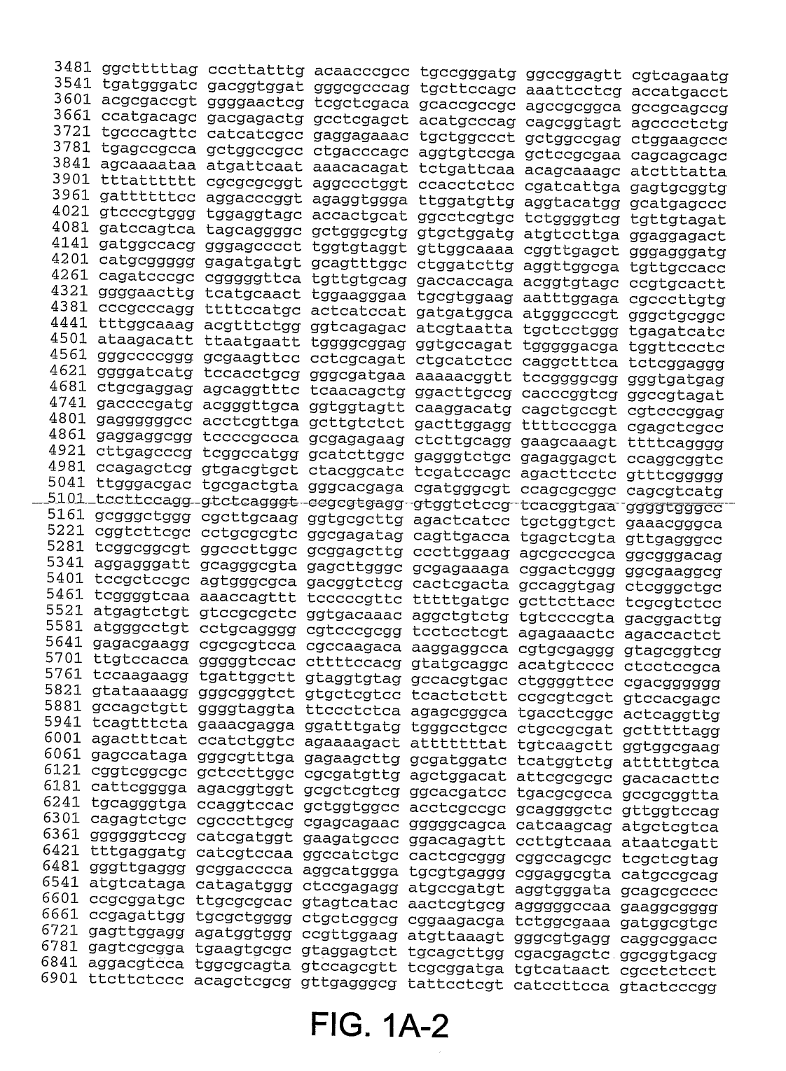

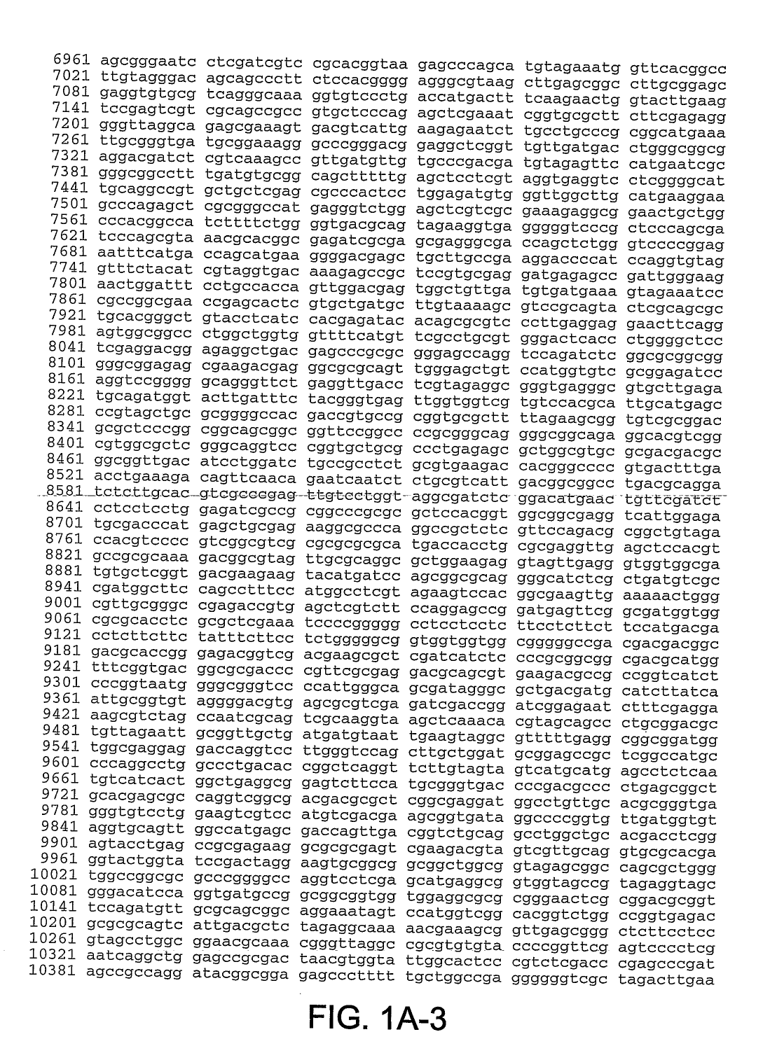

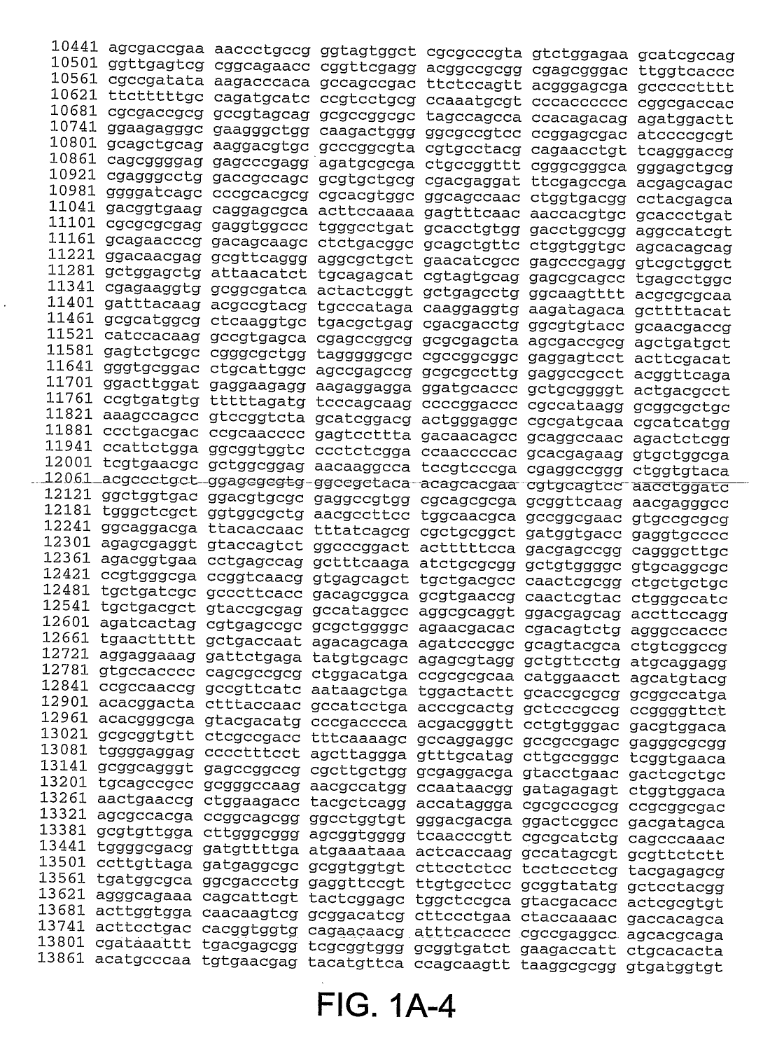

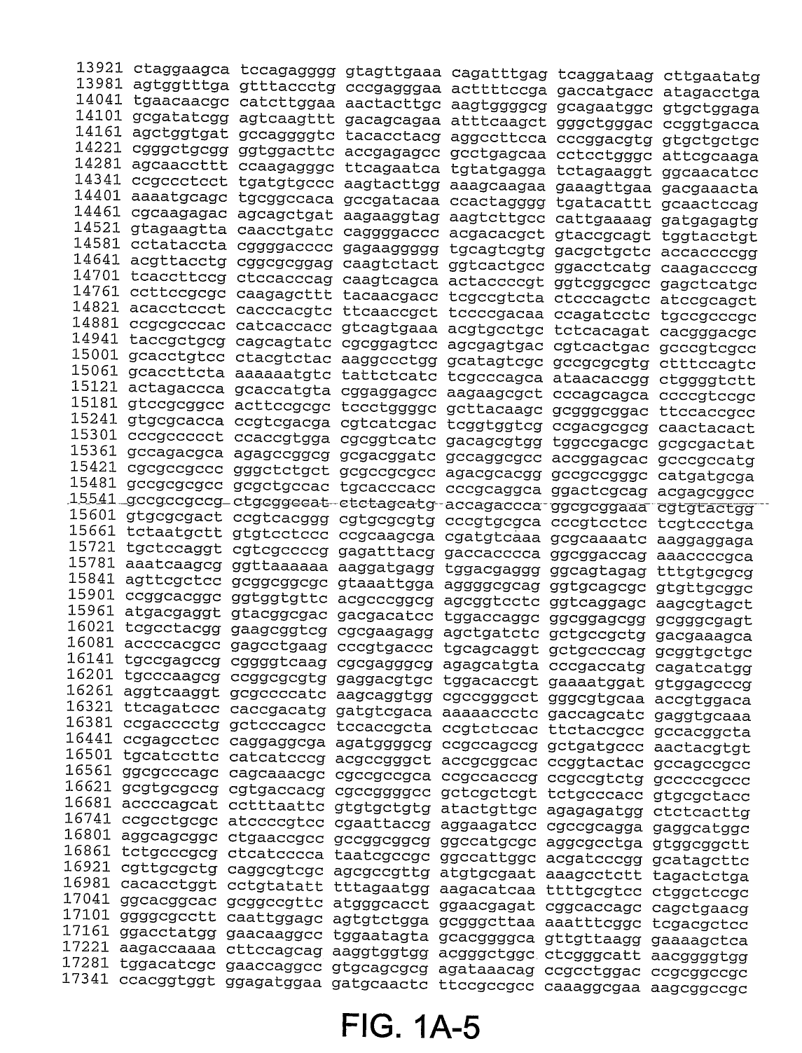

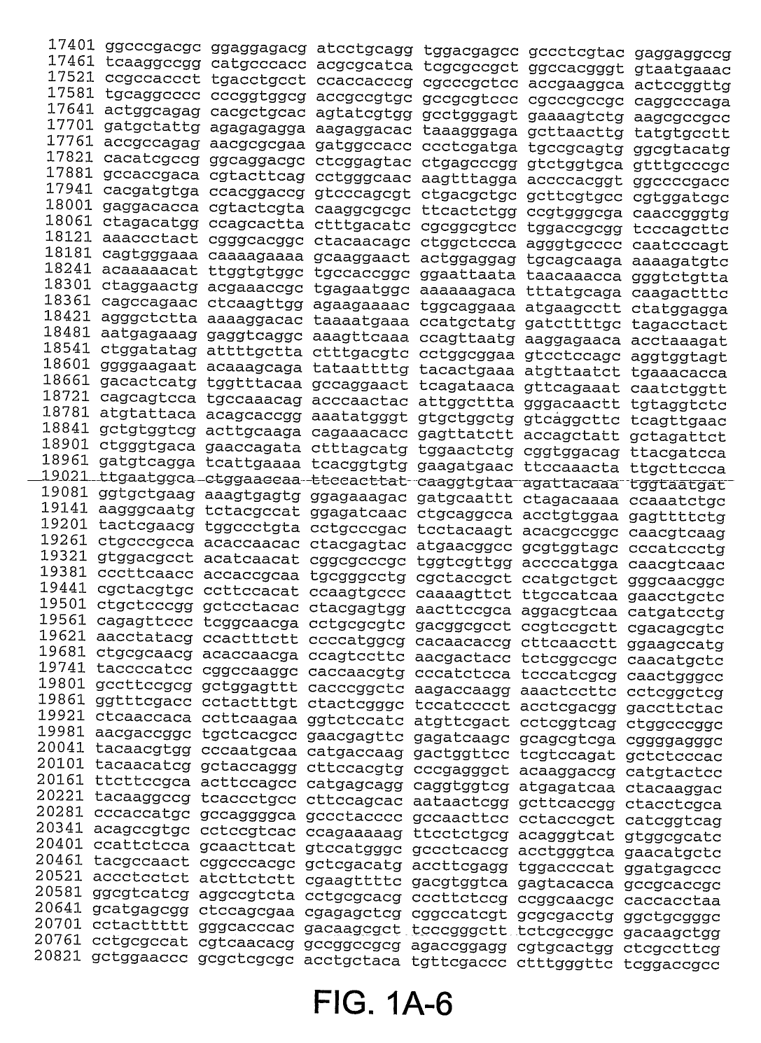

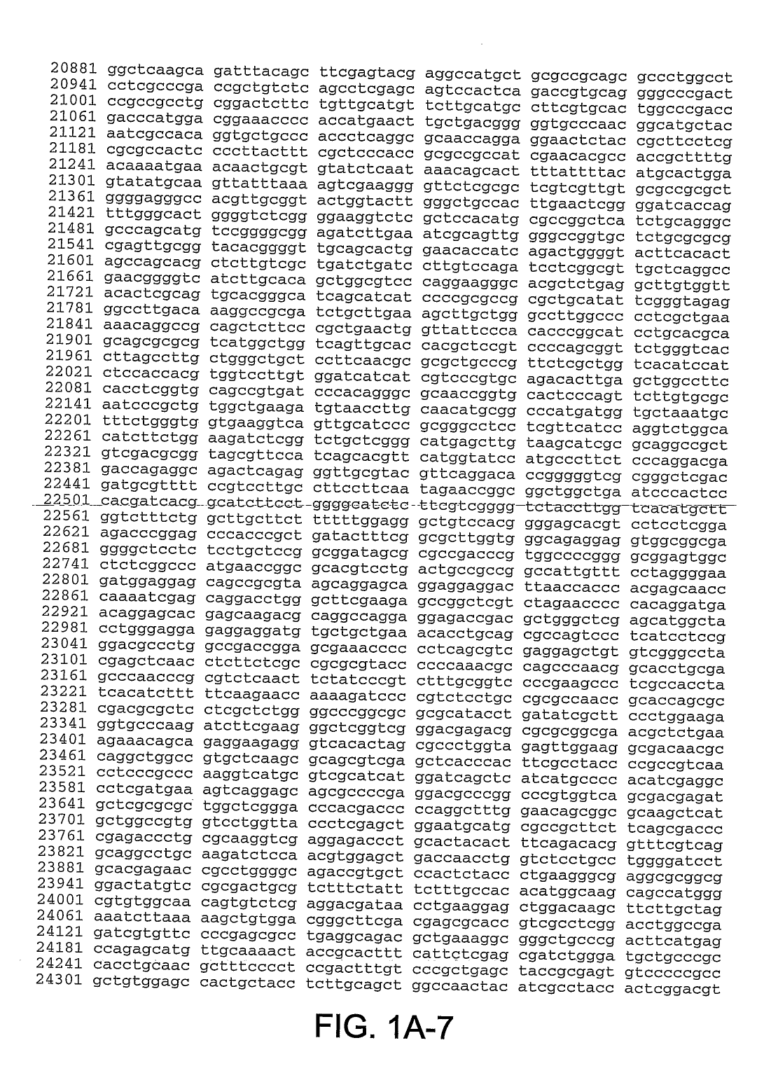

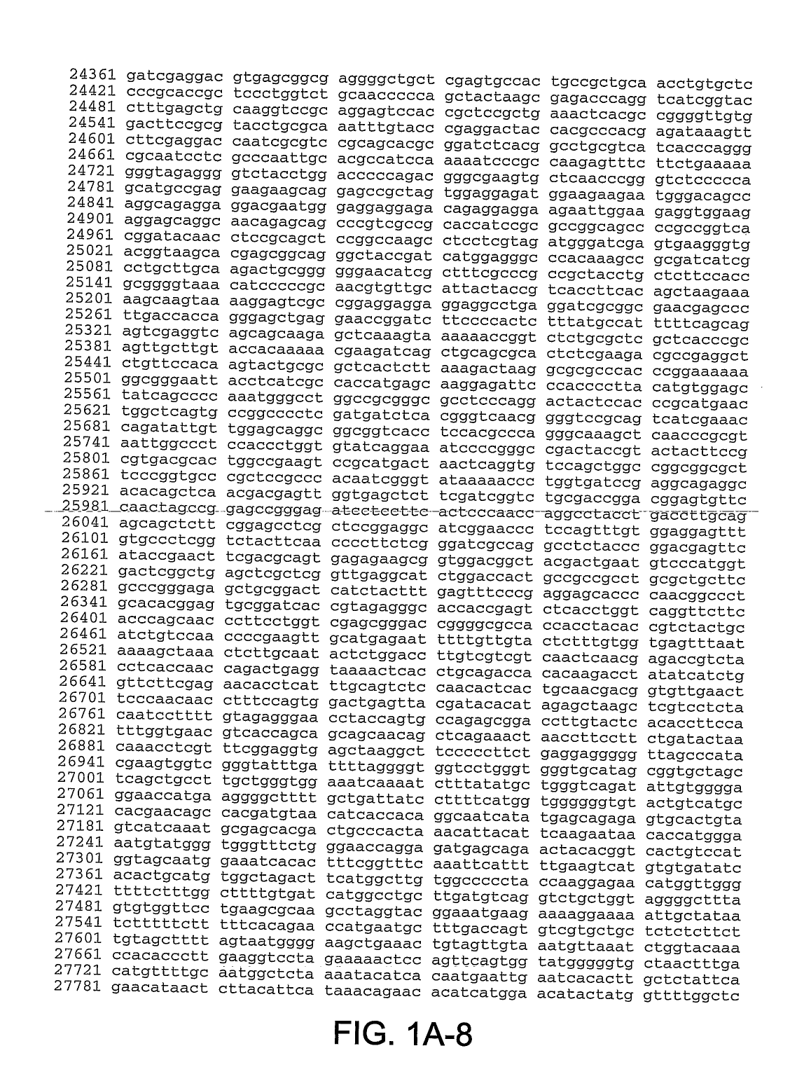

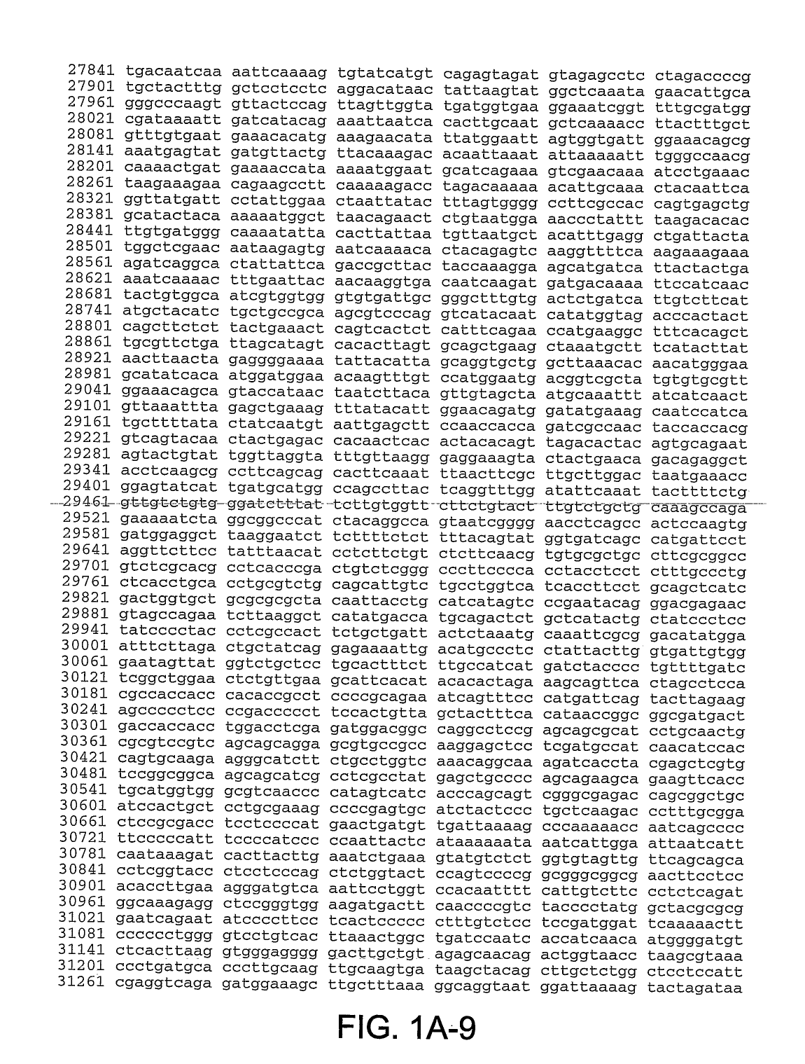

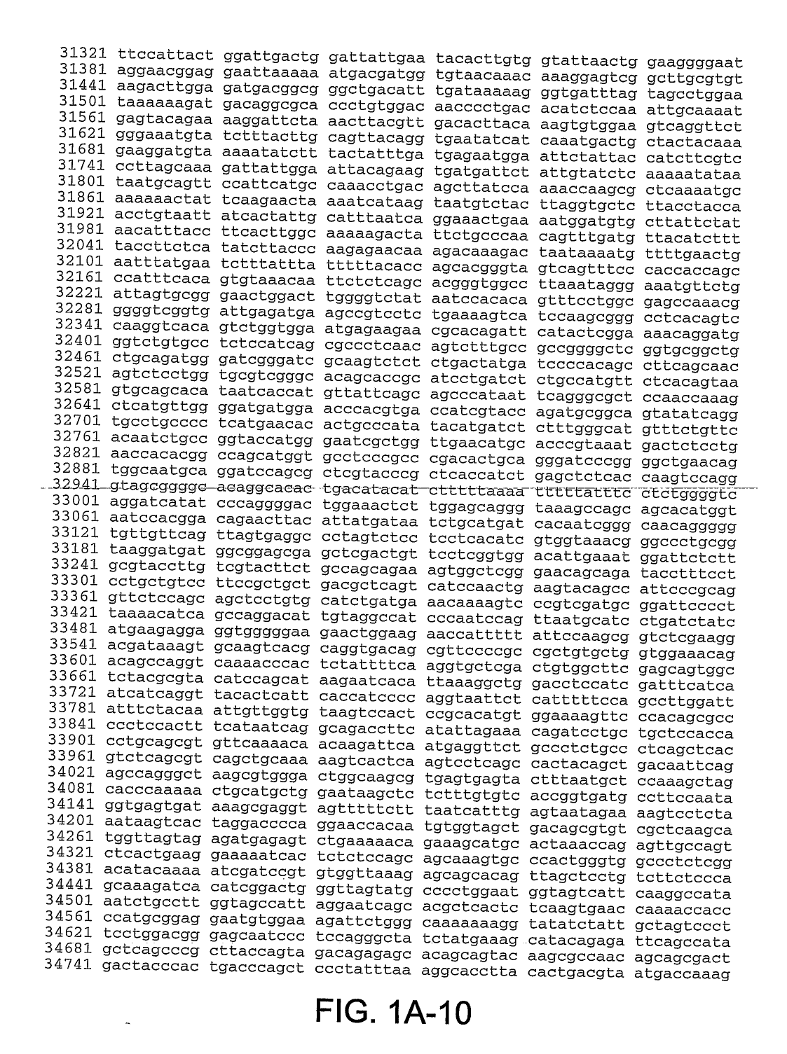

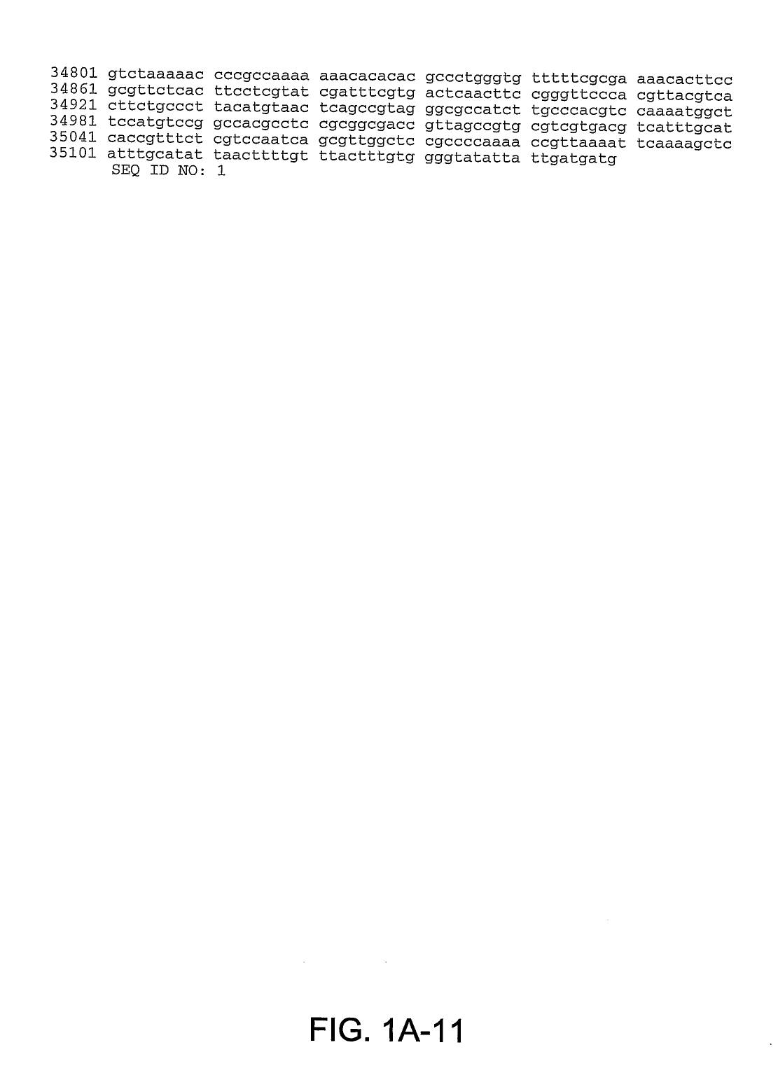

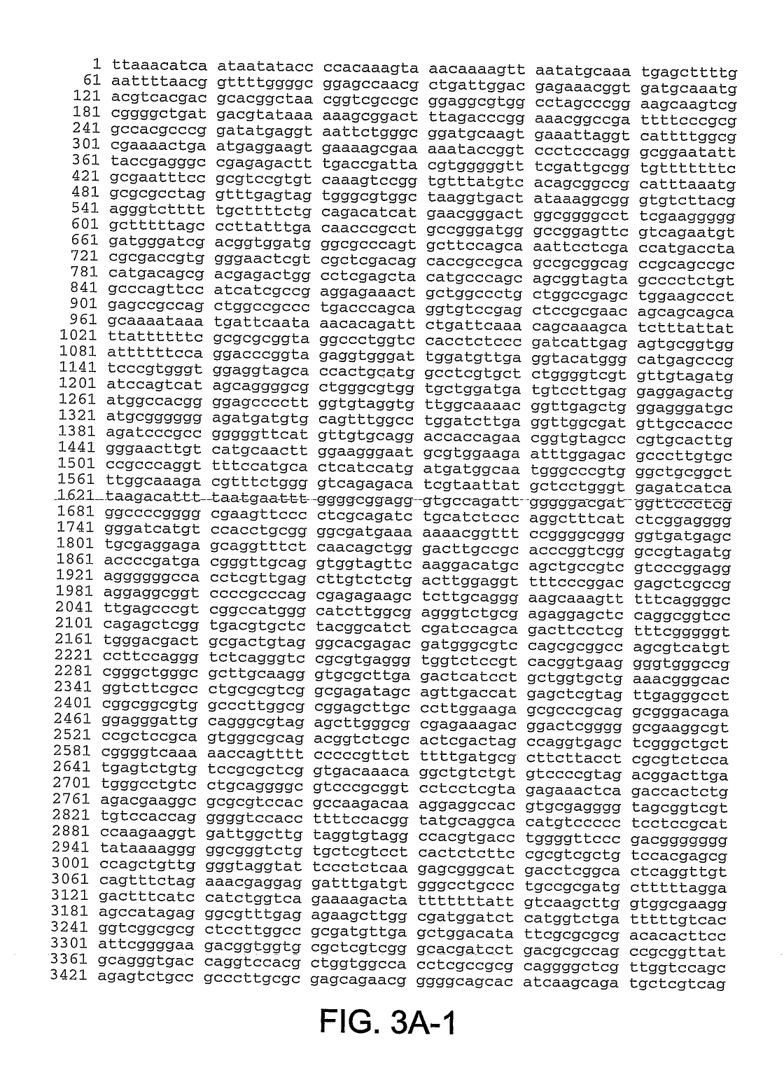

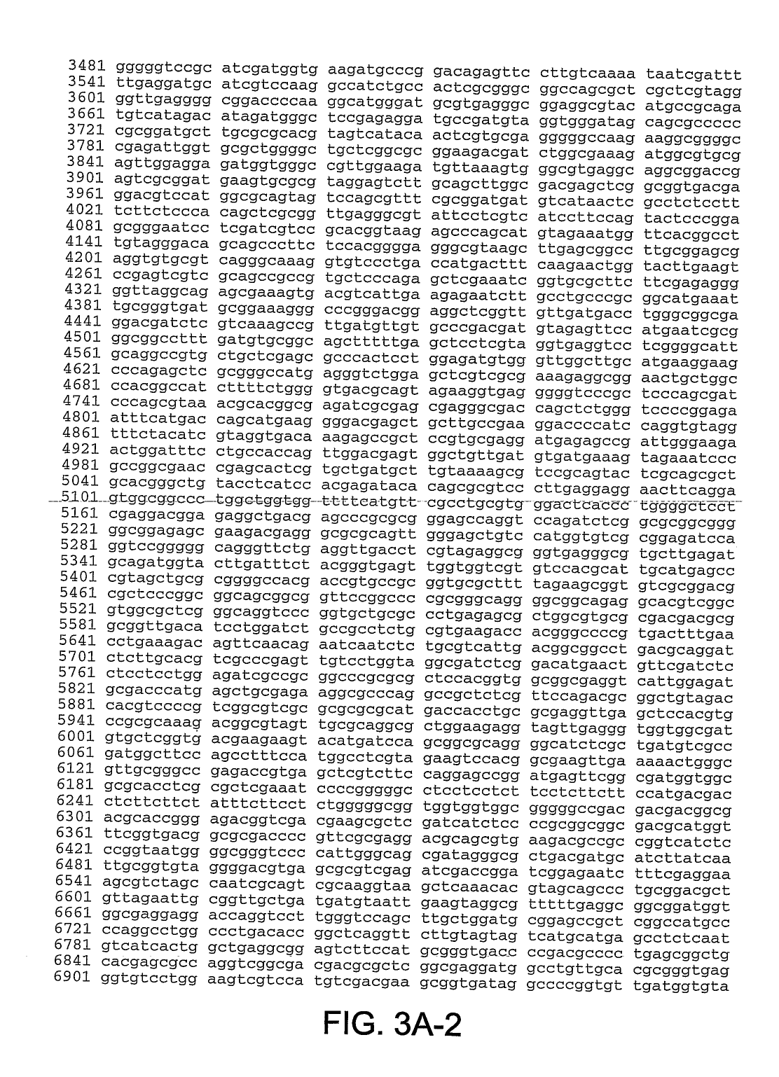

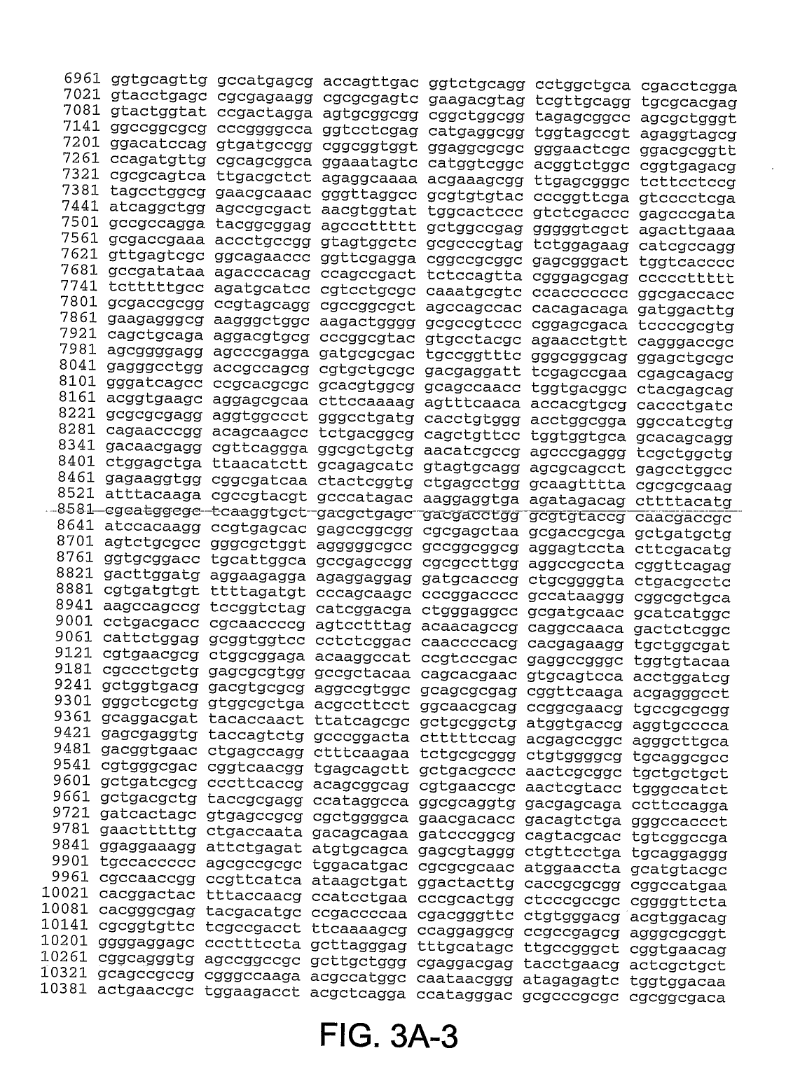

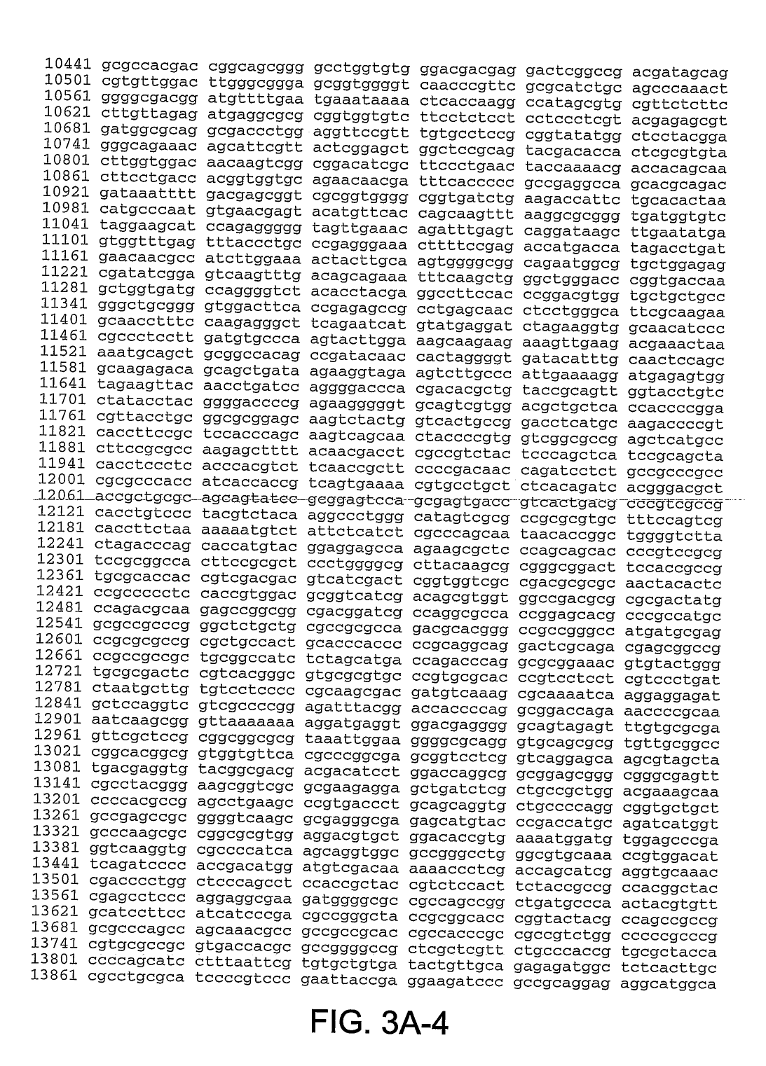

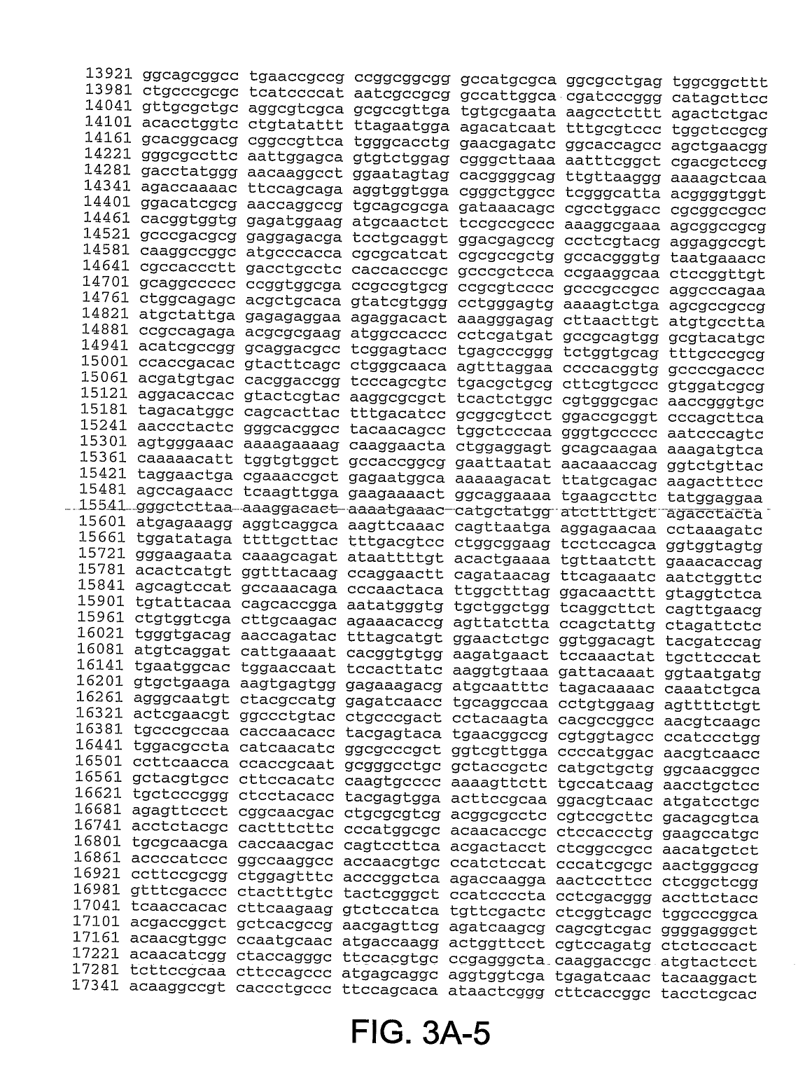

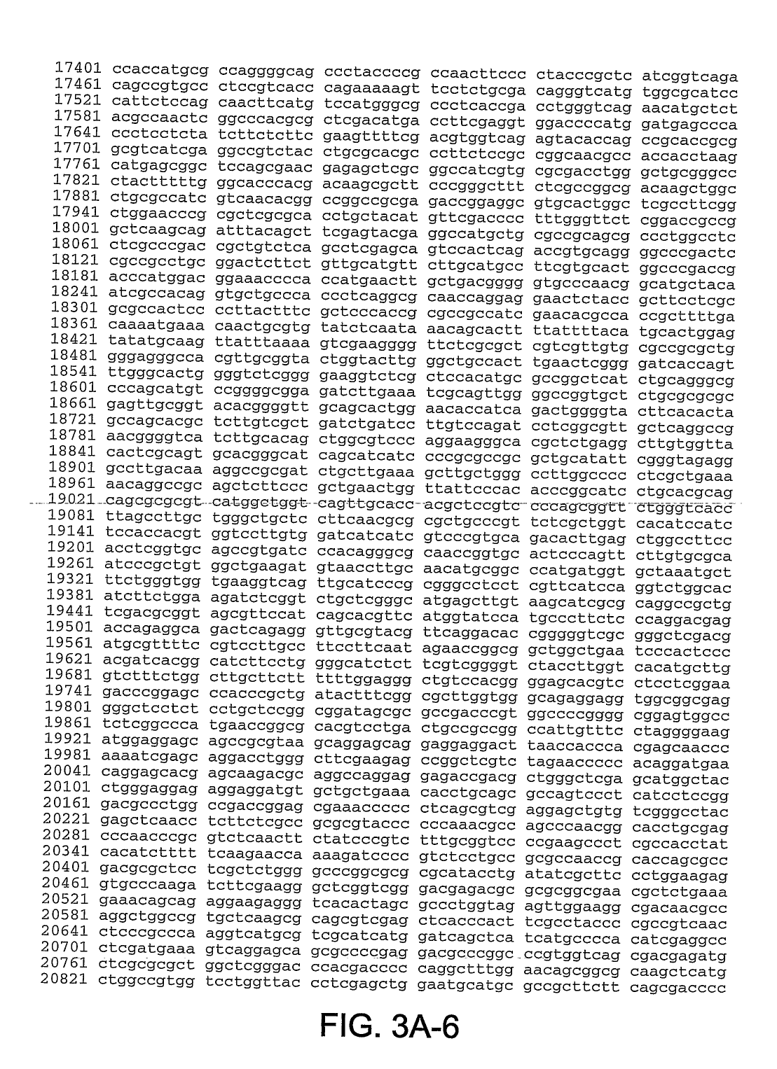

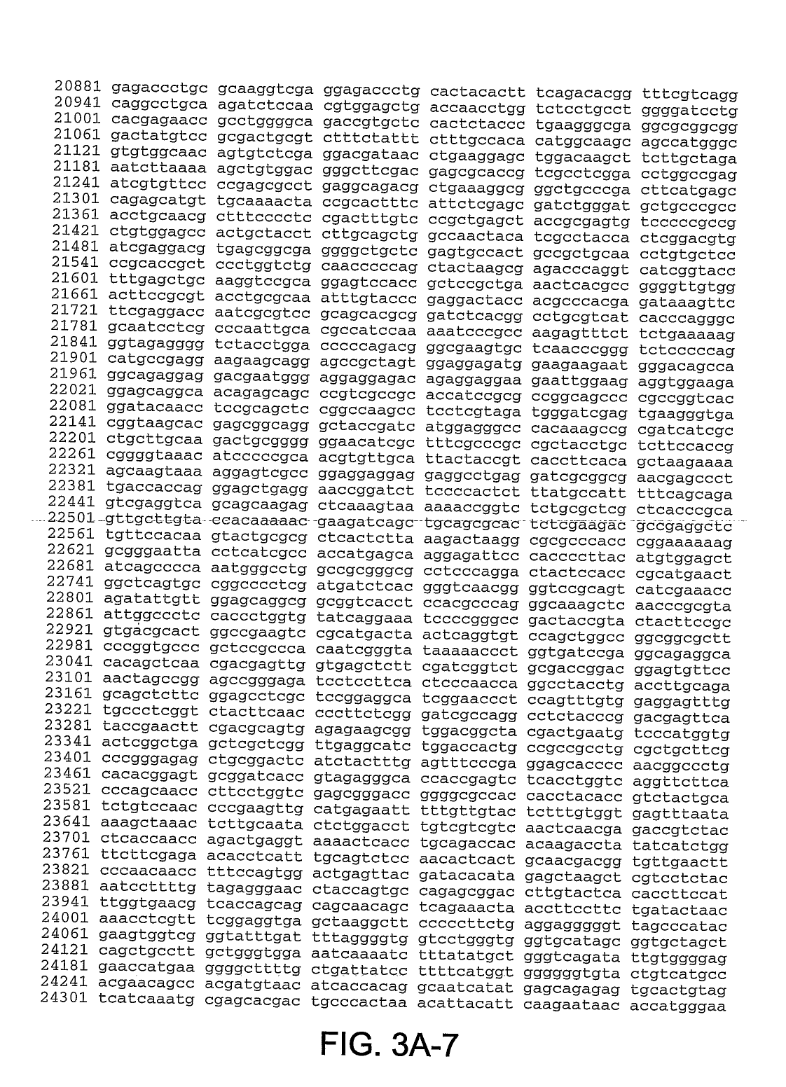

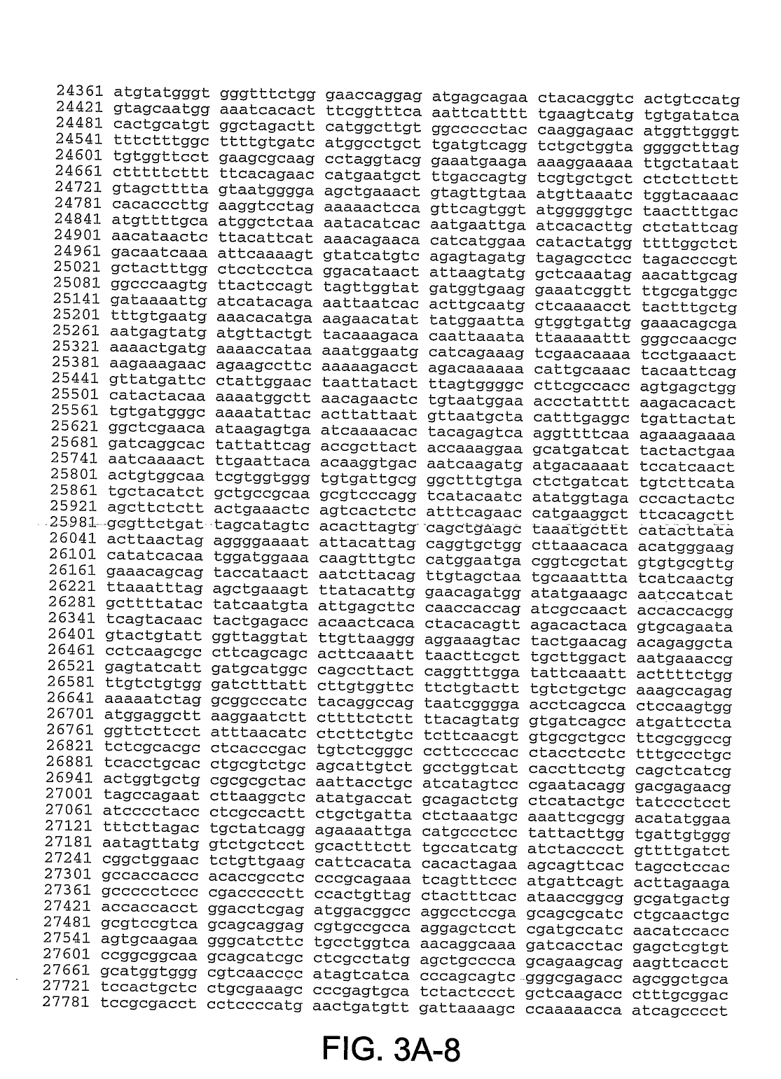

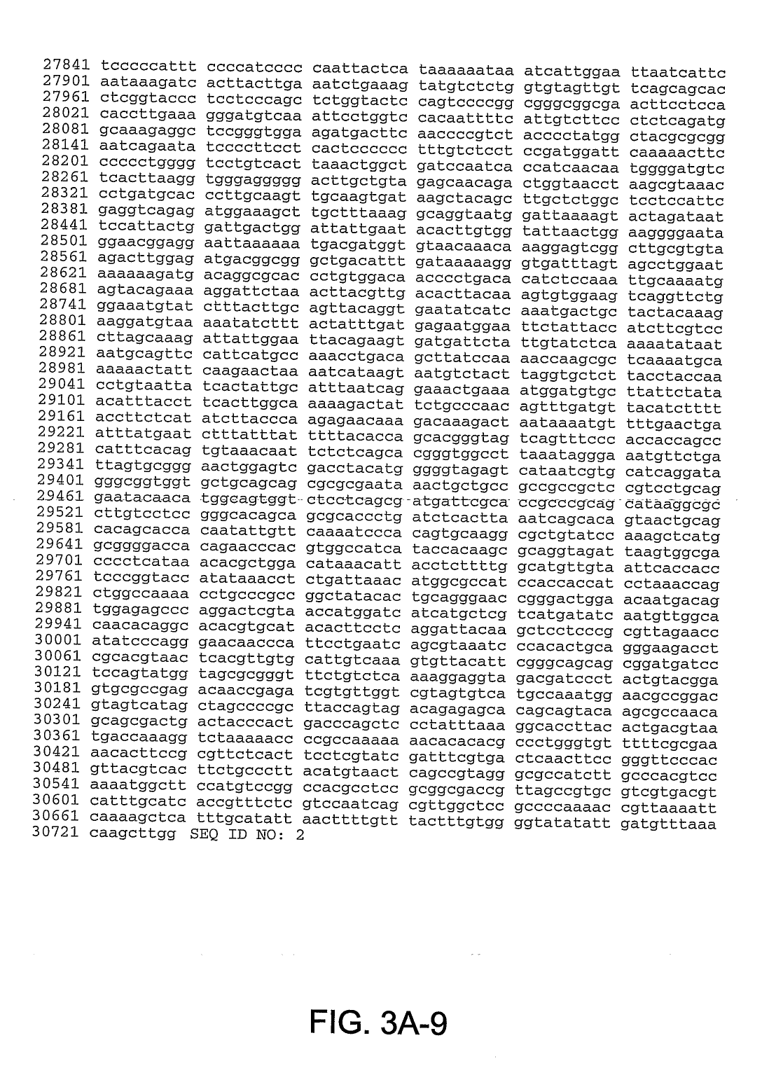

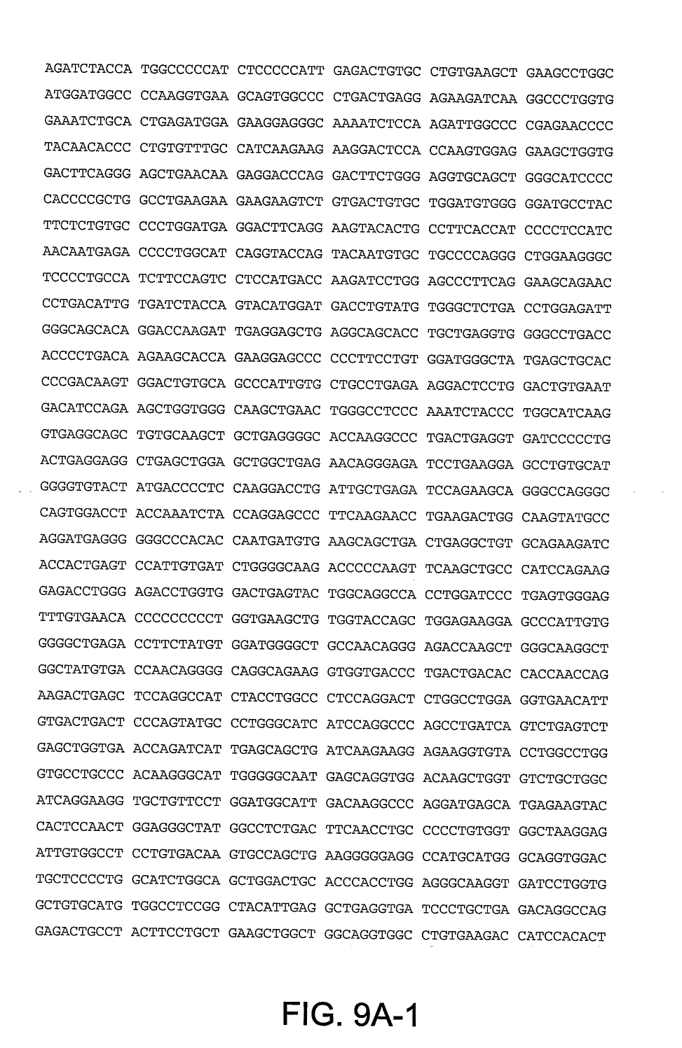

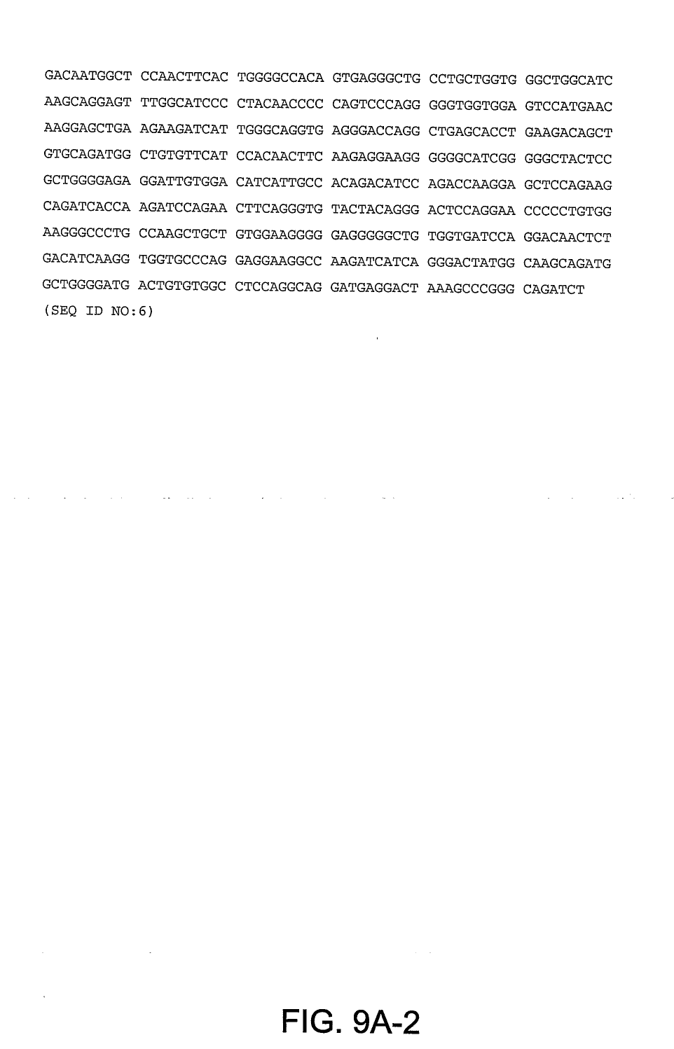

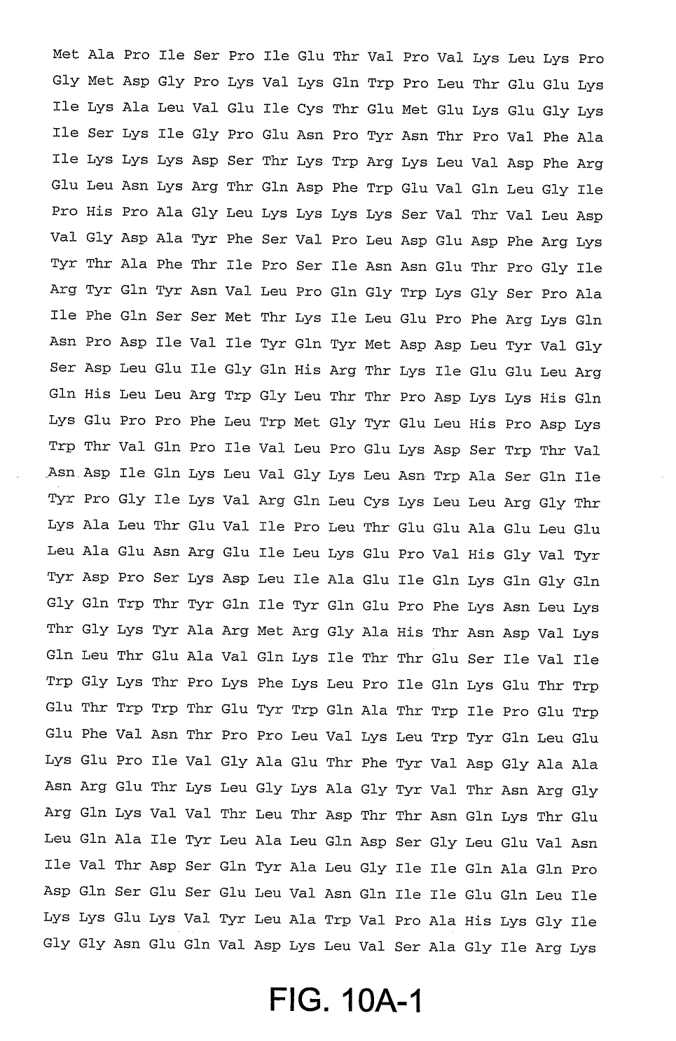

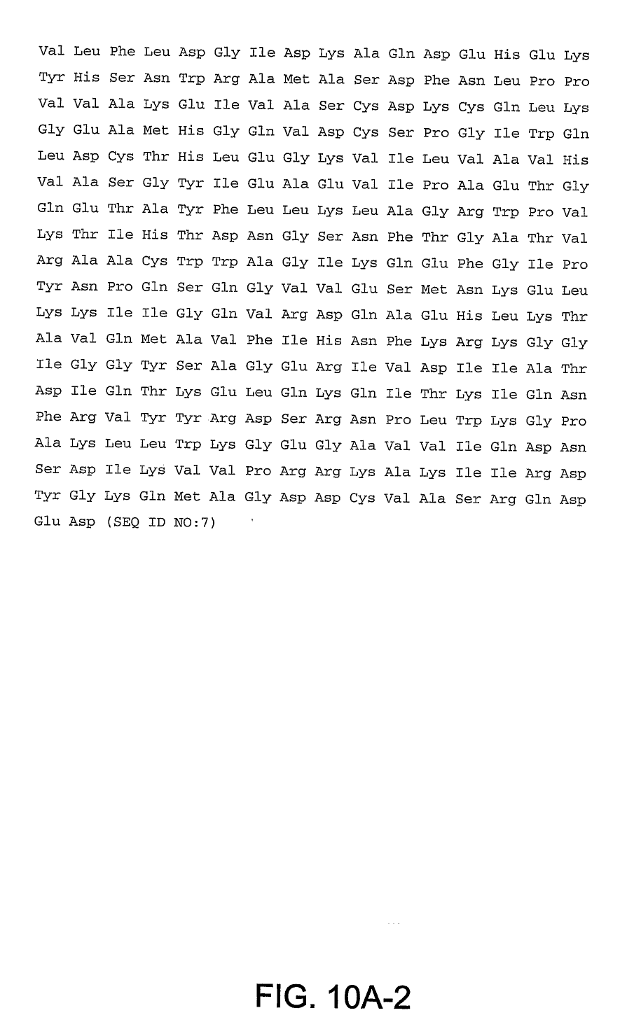

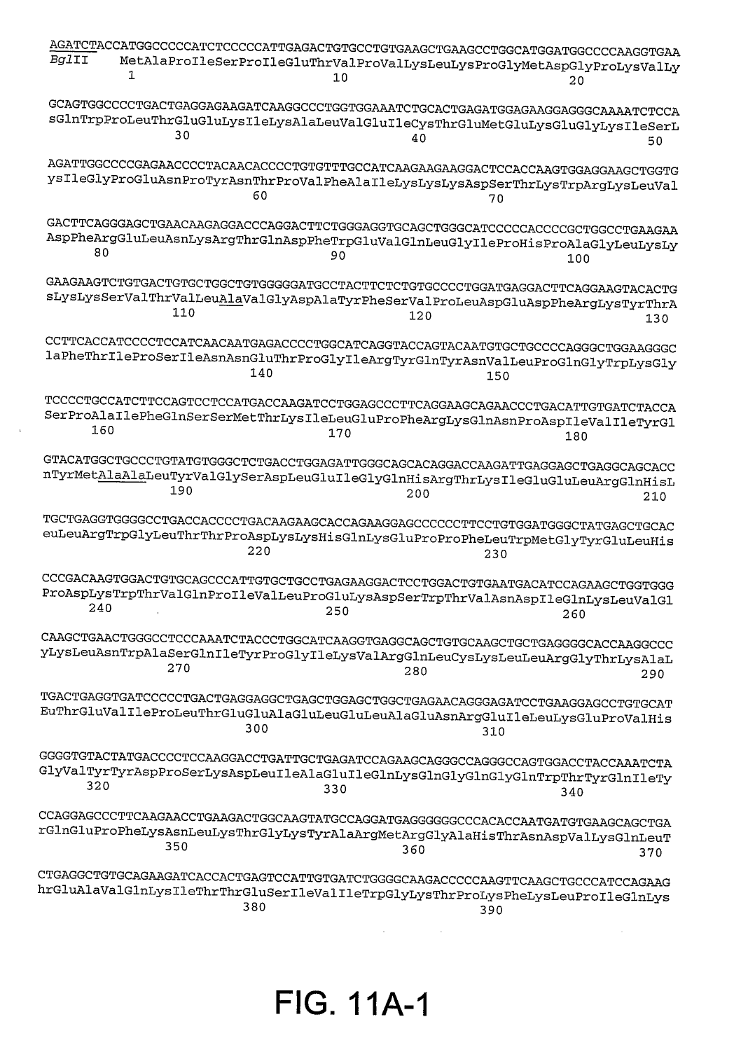

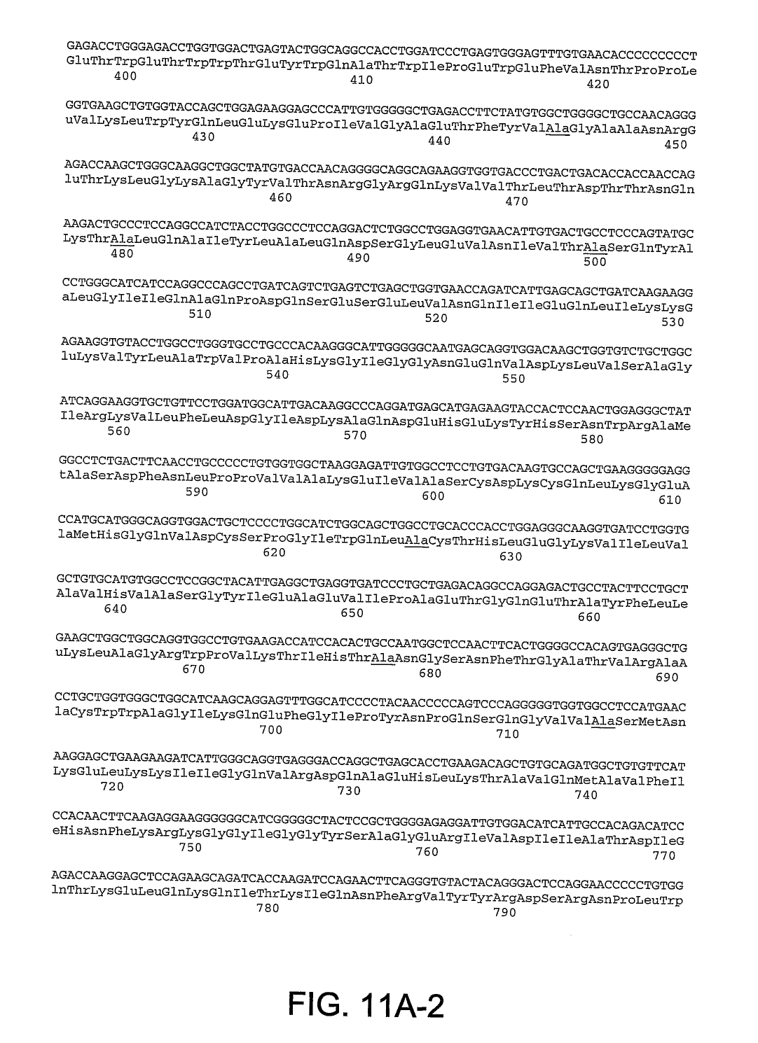

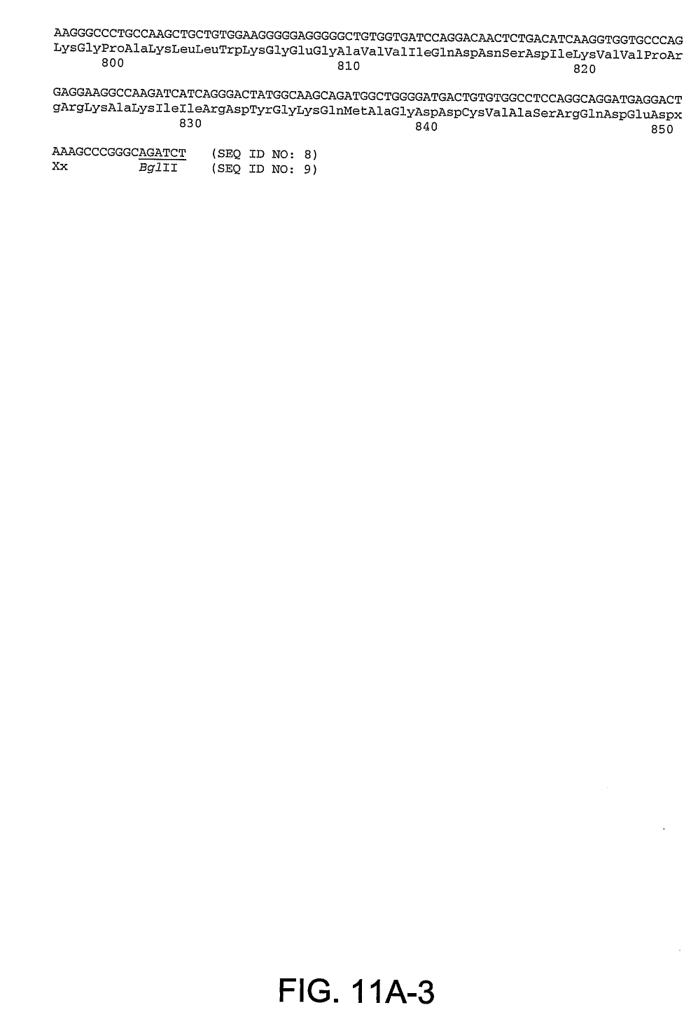

Accordingly, the present invention relates to recombinant adenoviral vectors of serotype 26 suitable for use in gene therapy or vaccination protocols. A nucleic acid sequence disclosed herein for adenovirus serotype 26 (SEQ ID NO: 1) is illustrated in FIGS. 1A-1 to 1A-11, although any functional homologue or different strain of adenovirus serotype 26 forms an embodiment hereof, and can be utilized in accordance with the vectors, methods and compositions of the present invention; as one of ordinary skill in the art will appreciate. Sequence variation within Ad26 sequence has been noted by Applicants. Accordingly, Ad26 vectors possessing sequence variation are encompassed as embodiments hereof. The following are some examples of sequence variation that can be found within Ad26 (base pair numbers in reference to SEQ ID NO: 1): (1) base pair 5972 as a “T” rather than a “G”; (2) base pair 7632 as a “G” rather than an “A”; (3) base pair 7668 as a “G” rather than a “T”; (4) base pair 11095 as a “G” rather than a “C”; (5) base pair 11560 as a “G” rather than an “A”; (6) base pair 12215 as a “G” rather than an “A”; (7) base pair 12296 as a “T” rather than a “G”; (8) base pair 12320 as a “G” rather than a “T”; (9) base pair 12357 as a “C” rather than a “T”; (10) base pair 12392 as a “C” rather than a “T”; (11) base pair 12437 as a “G” rather than an “A”; (12) base pair 12470 as an “A” rather than a “G”; (13) base pair 12539 as a “C” rather than a “T”; (14) base pair 12608 as a “G” rather than a “T”; (15) base pair 12734 as a “C” rather than a “T”; (16) base pair 12764 as an “A” rather than a “G”; (17) base pair 12767 as a “T” rather than a “C”; (18) base pair 12794 as a “T” rather than a “C”; (19) base pair 12842 as a “T” rather than a “C”; (20) base pair 12879 as a “C” rather than a “T”; (21) base pair 13038 as a “G” rather than an “A”; (22) base pairs 13085-13088 as “CCGC” rather than “GAGG”; (23) base pair 13094 as an “A” rather than a “C”; (24) base pair 13216 as a “T” rather than a “C”; (25) base pair 13448 as a “G” rather than an “A”; (26) base pair 15297 as a “T” rather than a “C”; (27) base pair 15300 as a “C” rather than a “T”; (28) base pair 16226 as an “A” rather than a “C”; (29) base pair 16237 as a “G” rather than a “C”; (30) base pair 16379 as a “G” rather than an “A”; (31) base pair 16897 as a “C” rather than a “T”; (32) base pair 19626 as a “C” rather than an “A”; (33) base pair 19662 as a “C” rather than a “T”; (34) base pair 19665 as a “C” rather than an “A”; (35) base pair 19669 as a “C” rather than a “T”; (36) base pair 19785 as a “C” rather than an “A”; (37) base pair 19848 as a “C” rather than a “T”; (38) base pair 19851 as a “T” rather than a “C”; (39) base pair 19857 as a “T” rather than a “C”; (40) base pair 20205 as an “A” rather than a “G”; (41) base pair 20253 as a “C” rather than a “T”; (42) base pair 20277 as a “G” rather than a “C”; (43) base pair 21598 as a “G” rather than an “A”; (44) base pair 21601 as a “G” rather than an “A”; (45) base pair 21757 as an “A” rather than a “1”; (46) base pair 21688 as a “G” rather than a “T”; (47) base pair 21790 as a “G” rather than an “A”; (48) base pair 22176 as a “G” rather than a “T”; (49) an additional 3 base pairs (TTC) between base pairs 22518 and 22519; (50) base pair 22567 as a “C” rather than a “T”; (51) base pair 22571 as an “A” rather than a “G”; (52) an additional 6 base pairs (GGCAGT) between base pairs 22582 and 22583; (53) base pair 22597 as a “T” rather than a “C”; (54) base pair 22605 as a “C” rather than a “G”; (55) base pair 22748 as a “T” rather than a “C”; (56) base pair 23206 as a “G” rather than an “A”; (57) base pair 26536 as an “A” rather than a “G”; and (58) base pairs 30217-30231 deleted.

One of skill in the art, provided with the sequence information disclosed herein, can furthermore identify other variants of adenovirus serotype 26 sequences. Serotype classification is well understood in the art. Adenovirus serotypes have been distinguished in the art via a number of art-appreciated biological, chemical, immunological and structural criteria including but not limited to hemagglutination properties of rat and rhesus monkey erythrocytes, DNA homology, restriction enzyme cleavage patterns, percentage G+C content and oncogenicity; Straus, supra; Horwitz, supra. A given serotype can be identified by any number of methods including, but not limited to, restriction mapping of viral DNA; analyzing mobility of viral DNA; analyzing mobility of virion polypeptides on SDS-polyacrylamide gels following electrophoresis; comparison of sequence information to known sequences particularly from capsid genes (e.g., hexons) which contain sequences that define a serotype; and comparison of sequence information with reference sera for a particular serotype available from the ATCC. Classification of adenovirus serotypes by SDS-PAGE has been discussed in Wadell et al., 1980 Ann. N.Y. Acad. Sci. 354:16-42. Classification of adenovirus serotypes by restriction mapping has been discussed in Wadell et al., Current Topics in Microbiology and Immunology 110: 191-220.

Adenovirus serotype 26 vectors in accordance with the present invention are at least partially deleted/mutated in E1 such that any resultant viruses are devoid (or essentially devoid) of E1 activity, rendering the vectors incapable of replication in the intended host. Preferably, the E1 region is completely deleted or inactivated. The adenoviruses may contain additional deletions in E3, and other early regions, albeit in situations where E2 and/or E4 is deleted, E2 and/or E4 complementing cell lines may be required to generate recombinant, replication-defective adenoviral vectors.

Adenoviral vectors of use in the methods of the present invention can be constructed using well known techniques, such as those reviewed in Graham & Prevec, 1991 In Methods in Molecular Biology Gene Transfer and Expression Protocols, (Ed. Murray, E. J.), p. 109; and Hitt et al., 1997 “Human Adenovirus Vectors for Gene Transfer into Mammalian Cells” Advances in Pharmacology 40:137-206.

E1-complementing cell lines used for the propagation and rescue of recombinant adenoviruses as described herein should provide elements essential for the viruses to replicate, whether the elements are encoded in the cell's genetic material or provided in trans. It is, furthermore, preferable that the E1-complementing cell lines and the vectors not contain overlapping elements which could enable homologous recombination between the nucleic acid of the vector and the nucleic acid of the cell line, potentially leading to replication competent virus (or replication competent adenovirus “RCA”). Often, propagation cells are human cells derived from the retina or kidney, although any cell line capable of expressing the appropriate E1 and any other critical deleted region(s) can be utilized to generate adenovirus suitable for use in the methods of the present invention. Embryonal cells such as amniocytes have been shown to be particularly suited for the generation of E1 complementing cell lines. Several cell lines are available and include but are not limited to the known cell lines PER.C6® (ECACC deposit number 96022940), 911, 293, and E1 A549. PER.C6® cell lines are described in WO 97/00326 (published Jan. 3, 1997) and issued U.S. Pat. No. 6,033,908. PER.C6® is a primary human retinoblast cell line transduced with an E1 gene segment that complements the production of replication defective (FG) adenovirus, but is designed to prevent generation of replication competent adenovirus by homologous recombination. 293 cells are described in Graham et al., 1977 J. Gen. Virol. 36:59-72. For the propagation and rescue of non-group C adenoviral vectors, a cell line expressing an E1 region which is complementary to the E1 region deleted in the viruses being propagated can be utilized. For example, a specific example of cells suitable for the propagation of recombinant Ad26 E1-deleted vectors express the early region 1 (E1) of adenovirus 26 or another group D serotype. Alternatively, a cell line expressing regions of E1 and E4 derived from the same serotype can be employed; see, e.g., U.S. Pat. No. 6,270,996. Another alternative would be to propagate non-group C adenovirus in available E1-expressing cell lines (e.g., PER.C6®, A549 or 293). This latter method involves the incorporation of a critical E4 region into the adenovirus to be propagated. The critical E4 region is native to a virus of the same or highly similar serotype as that of the E1 gene product(s) (particularly the E1B 55K region) of the complementing cell line, and comprises typically, at a minimum, E4 open reading frame 6 (“ORF6”); see PCT/US2003/026145, published Mar. 4, 2004. One of skill in the art can readily appreciate and carry out numerous other methods suitable for the production of recombinant, replication-defective adenoviruses of use in the methods of the present invention. Following viral production in whatever means employed, viruses may be purified, formulated and stored prior to host administration.

Such methods for producing recombinant, replication-defective adenoviruses of serotype 26 are considered part of the present invention. Particularly, such methods comprising the acts of (1) introducing recombinant, replication-defective adenoviral vectors of serotype 26 into appropriate adenoviral E1-complementing cells and (2) allowing for the production of viral particles. Viral particles so produced and host cells comprising recombinant, replication-defective adenoviral serotype 26 vectors of the present invention form additional aspects of the present invention. “Isolated host cells” are defined herein as a population of cells not including a transgenic human being.

Adenoviral vectors in accordance with the present invention are very well suited to effectuate the expression of heterologous polypeptides, especially in situations where an individual's immune response effectively prevents administration or readministration via the more commonly employed adenoviral serotypes. Accordingly, specific embodiments of the present invention are recombinant, replication-defective adenoviral vectors of serotype 26 which comprise a heterologous nucleic acid encoding a polypeptide(s) of interest. The expressed nucleic acid can be DNA and/or RNA, and can be double or single stranded. The nucleic acid can be inserted in an E1 parallel (transcribed 5′ to 3′ relative to the vector backbone) or anti-parallel (transcribed 3′ to 5′ relative to the vector backbone) orientation. The nucleic acid can be codon-optimized for expression in the desired host (e.g., a mammalian host). The heterologous nucleic acid can be in the form of an expression cassette. A gene expression cassette can contain (a) nucleic acid encoding a protein or antigen of interest; (b) a heterologous promoter operatively linked to the nucleic acid encoding the protein/antigen; and (c) a transcription termination signal.

In specific embodiments, the heterologous promoter is recognized by a eukaryotic RNA polymerase. One example of a promoter suitable for use in the present invention is the immediate early human cytomegalovirus promoter (Chapman et al., 1991 Nucl. Acids Res. 19:3979-3986). Further examples of promoters that can be used in the present invention are the strong immunoglobulin promoter, the EFI alpha promoter, the murine CMV promoter, the Rous Sarcoma Virus promoter, the SV40 early/late promoters and the beta actin promoter, albeit those of skill in the art can appreciate that any promoter capable of effecting expression of the heterologous nucleic acid in the intended host can be used in accordance with the methods of the present invention. The promoter may comprise a regulatable sequence such as the Tet operator sequence. Sequences such as these that offer the potential for regulation of transcription and expression are useful in circumstances where repression/modulation of gene transcription is sought. The adenoviral gene expression cassette may comprise a transcription termination sequence; specific embodiments of which are the bovine growth hormone termination/polyadenylation signal (bGHpA) or the short synthetic polyA signal (SPA) of 49 nucleotides in length defined as follows: AATAAAAGATCTTTATTTTCATTAGATCTGTGTGTTGGTTTTTTGTGTG (SEQ ID NO: 5). A leader or signal peptide may also be incorporated into the transgene. In specific embodiments, the leader is derived from the tissue-specific plasminogen activator protein, tPA.

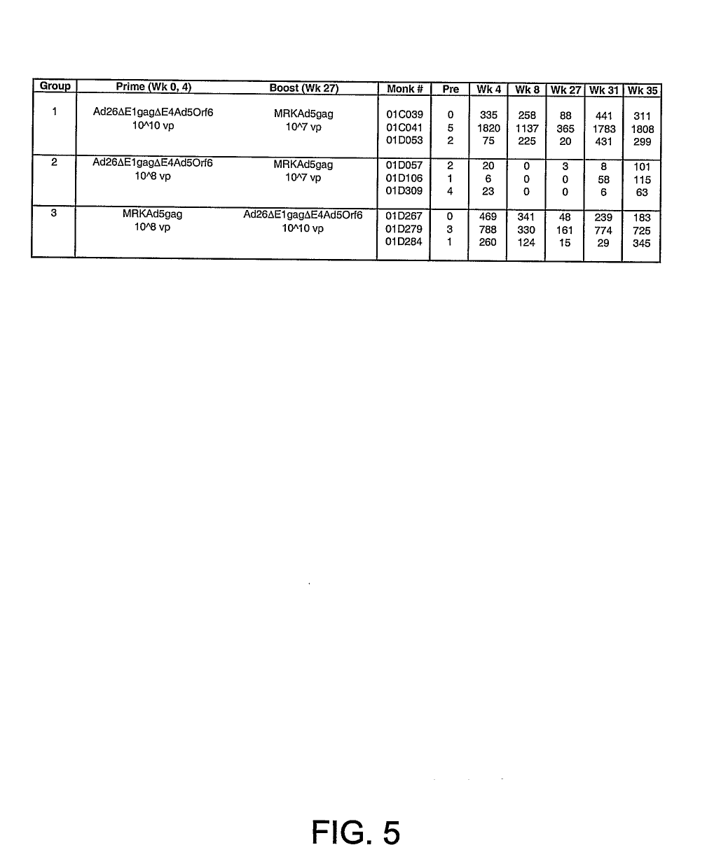

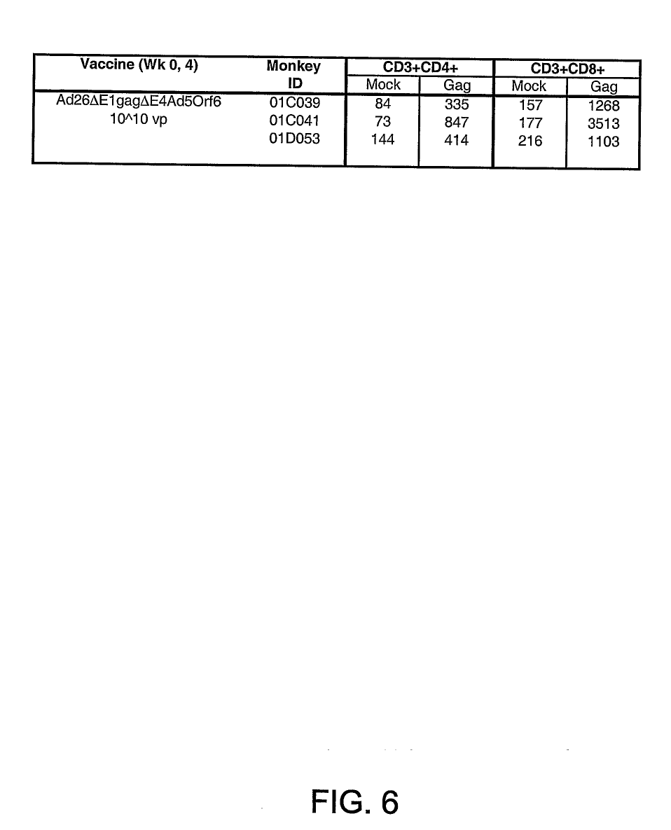

Heterologous nucleic acids of interest typically encode immunogenic and/or therapeutic proteins. Preferred therapeutic proteins are those which elicit some measurable therapeutic benefit in the individual host upon administration. Preferred immunogenic proteins are those proteins which are capable of eliciting a protective and/or beneficial immune response in an individual. A specific embodiment of the present invention, illustrated herein, is the delivery of nucleic acid encoding representative immunogenic proteins (HIV Gag, Nef and/or Pol) by the vectors, methods and compositions disclosed, albeit any gene encoding a therapeutic or immunogenic protein can be used in accordance with the methods disclosed herein and form important embodiments hereof. The vectors, methods and compositions of the present invention can be used to effectuate the delivery of any polypeptide whose presence/function brings about a desired effect in a given host, particularly a therapeutic/immunogenic effect useful in the treatment/alteration/modification of various conditions associated with, caused by, effected by (positively or negatively), exacerbated by, or modified by the presence or absence of a particular nucleic acid, protein, antigen, fragment, or activity associated with any of the foregoing. Adenovirus serotype 26 vectors were found to induce significant levels of gag-specific T cells; FIG. 5. Moreover, the results indicated that immunization with the disclosed vectors was able to elicit both HIV-specific CD4+ and CD8+ T cells; FIG. 6. These results were particularly manifested when Ad26 was used in a prime-boost protocol with an adenoviral vector of serotype 5.

One aspect of the present invention, therefore, relates to adenovirus serotype 26-based vectors carrying heterologous nucleic acid encoding an HIV antigen(s)/protein(s), vector compositions, and methods of using same. Human Immunodeficiency Virus (“HIV”) is the etiological agent of acquired human immune deficiency syndrome (AIDS) and related disorders. HIV is an RNA virus of the Retroviridae family and exhibits the 5′LTR-gag-pol-env-LTR 3′ organization of all retroviruses. The integrated form of HIV, known as the provirus, is approximately 9.8 Kb in length. Each end of the viral genome contains flanking sequences known as long terminal repeats (LTRs).

Heterologous nucleic acid encoding an HIV antigen/protein may be derived from any HIV strain including, but not limited to, HIV-1 and HIV-2, strains A, B, C, D, E, F, G, H, I, O, IIIB, LAV, SF2, CM235, and US4; see, e.g., Myers et al., eds. “Human Retroviruses and AIDS”: 1995 (Los Alamos National Laboratory, Los Alamos N. Mex. 97545). Another IV strain suitable for use in the methods disclosed herein is HIV-1 strain CAM-1; Myers et al, eds. “Human Retroviruses and AIDS”: 1995, IIA3-IIA19. This gene closely resembles the consensus amino acid sequence for the clade B (North American/European) sequence. IV gene sequence(s) may be based on various clades of HIV-1; specific examples of which are Clades A, B, and C. Sequences for genes of many IV strains are publicly available from GenBank and primary, field isolates of HIV are available from the National Institute of Allergy and Infectious Diseases (NIAID) which has contracted with Quality Biological (Gaithersburg, Md.) to make these strains available. Strains are also available from the World Health Organization (WHO), Geneva Switzerland.

HIV genes encode at least nine proteins and are divided into three classes; the major structural proteins (Gag, Pol, and Env), the regulatory proteins (Tat and Rev); and the accessory proteins (Vpu, Vpr, Vif and Nef). The gag gene encodes a 55-kilodalton (kDa) precursor protein (p55) which is expressed from the unspliced viral mRNA and is proteolytically processed by the HIV protease, a product of the pol gene. The mature p55 protein products are p17 (matrix), p24 (capsid), p9 (nucleocapsid) and p6. The pol gene encodes proteins necessary for virus replication-protease (Pro, P10), reverse transcriptase (RT, P50), integrase (IN, p31) and RNAse H (RNAse, p15) activities. These viral proteins are expressed as a Gag or Gag-Pol fusion protein which is generated by a ribosomal frame shift. The 55 kDa gag and 160 kDa gagpol precursor proteins are then proteolytically processed by the virally encoded protease into their mature products. The nef gene encodes an early accessory HIV protein (Nef) which has been shown to possess several activities such as down regulating CD4 expression, disturbing T-cell activation and stimulating HIV infectivity. The env gene encodes the viral envelope glycoprotein that is translated as a 160-kilodalton (kDa) precursor (gp160) and then cleaved by a cellular protease to yield the external 120-kDa envelope glycoprotein (gp120) and the transmembrane 41-kDa envelope glycoprotein (gp41). Gp120 and gp41 remain associated and are displayed on the viral particles and the surface of HIV-infected cells. The tat gene encodes a long form and a short form of the Tat protein, a RNA binding protein which is a transcriptional transactivator essential for HIV replication. The rev gene encodes the 13 kDa Rev protein, a RNA binding protein. The Rev protein binds to a region of the viral RNA termed the Rev response element (RRE). The Rev protein promotes transfer of unspliced viral RNA from the nucleus to the cytoplasm. The Rev protein is required for HIV late gene expression and in turn, HIV replication.

Nucleic acid encoding any HIV antigen may be utilized in the methods and compositions of the present invention (specific examples of which include but are not limited to the aforementioned genes, nucleic acid encoding active and/or immunogenic fragments thereof, and/or modifications/derivatives of any of the foregoing). The present invention contemplates as well the various codon-optimized forms of nucleic acid encoding HIV antigens, including codon-optimized HIV gag (including but by no means limited to p55 versions of codon-optimized full length (“FL”) Gag and tPA-Gag fusion proteins), HIV pol, HIV nef, HIV env, HIV tat, HIV rev, and modifications/derivatives of immunological relevance. Embodiments exemplified herein employ nucleic acid encoding codon-optimized Nef antigens; codon-optimized p55 Gag antigens; and codon-optimized Pol antigens. Codon-optimized HIV-1 gag genes are disclosed in PCT International Application PCT/US00/18332, published Jan. 11, 2001 (WO 01/02607). Codon-optimized HIV-1 env genes are disclosed in PCT International Applications PCT/US97/02294 and PCT/US97/10517, published Aug. 28, 1997 (WO 97/31115) and Dec. 24, 1997 (WO 97/48370), respectively. Codon-optimized HIV-1 pol genes are disclosed in U.S. application Ser. No. 09/745,221, filed Dec. 21, 2000 and PCT International Application PCT/US00/34724, also filed Dec. 21, 2000. Codon-optimized HIV-1 nef genes are disclosed in U.S. application Ser. No. 09/738,782, filed Dec. 15, 2000 and PCT International Application PCT/US00/34162, also filed Dec. 15, 2000. It is well within the purview of the skilled artisan to choose an appropriate nucleotide sequence including but not limited to those cited above which encodes a specific HIV antigen, or immunologically relevant portion or modification/derivative thereof. “Immunologically relevant”, “immunogenic” or “antigenic” as defined herein means (1) with regard to a viral antigen, that the protein is capable, upon administration, of eliciting a measurable immune response within an individual sufficient to retard the propagation and/or spread of the virus and/or to reduce/contain viral load within the individual; or (2) with regards to a nucleotide sequence, that the sequence is capable of encoding for a protein capable of the above. One of skill in the art can, furthermore, appreciate that any nucleic acid encoding for a protein, antigen, derivative or fragment capable of effectuating a desired result (sequences that may or may not be codon-optimized) is of use in the methods and compositions of the instant invention.

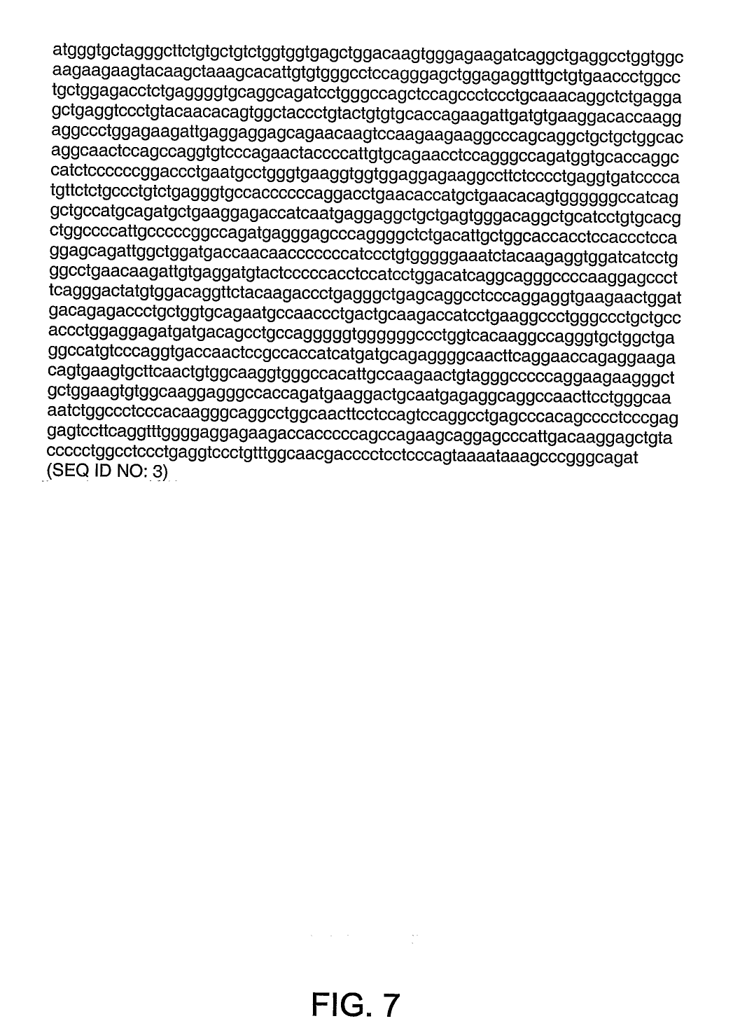

An example of a codon-optimized gag gene that can be utilized in the vectors, methods and compositions of the present invention is that disclosed in PCT/US00/18332, published Jan. 11, 2001 (see FIG. 7; SEQ ID NO: 3). The sequence is derived from HIV-1 strain CAM-1 and encodes full-length p55 gag. The gag gene of HIV-1 strain CAM-1 was selected as it closely resembles the consensus amino acid sequence for the clade B (North American/European) sequence (Los Alamos HIV database). The sequence was designed to incorporate human preferred (“humanized”) codons in order to maximize in vivo mammalian expression (Lathe, 1985, J. Mol. Biol. 183:1-12).

Open reading frames for various synthetic pol genes comprising coding sequences for reverse transcriptase (or RT which consists of a polymerase and RNase H activity) and integrase (IN) are disclosed in PCT/US00/34724. The protein sequences therein are based on Hxb2r, a clonal isolate of IIIB. This sequence has been shown to be closest to the consensus clade B sequence with only 16 nonidentical residues out of 848 (Korber, et al., 1998, Human retroviruses and AIDS, Los Alamos National Laboratory, Los Alamos, N. Mex.).

A particular embodiment of this portion of the invention relates to vectors comprising codon optimized nucleotide sequences which encode wt-pol constructs (herein, “wt-pol” or “wt-pol (codon optimized)”) wherein sequences encoding the protease (PR) activity are deleted, leaving codon optimized “wild type” sequences which encode RT (reverse transcriptase and RNase H activity) and IN integrase activity. A DNA molecule which encodes this protein is disclosed herein as SEQ ID NO: 6 (FIGS. 9A-1 to 9A-2), the open reading frame being contained from an initiating Met residue at nucleotides 10-12 to a termination codon from nucleotides 2560-2562. The open reading frame of the wild type pol construct (SEQ ID NO: 7; FIGS. 10A-1 to 10A-2) contains 850 amino acids.

Alternative specific embodiments relate to vectors comprising codon optimized HIV-1 pol wherein, in addition to deletion of the portion of the wild type sequence encoding the protease activity, a combination of active site residue mutations are introduced which are deleterious to HIV-1 pol (RT-RH-IN) activity of the expressed protein. Therefore, the present invention relates to adenoviral vectors comprising HIV-1 pol devoid of sequences encoding PR activity and containing a mutation(s) which at least partially, and preferably substantially, abolishes RT, RNase and/or IN activity. One type of HIV-1 pol mutant which is part and parcel of an adenoviral vector construct of use in the methods and compositions disclosed herein may include but is not limited to a mutated nucleic acid molecule comprising at least one nucleotide substitution which results in a point mutation which effectively alters an active site within the RT, RNase and/or IN regions of the expressed protein, resulting in at least substantially decreased enzymatic activity for the RT, RNase H and/or IN functions of HIV-1 Pol. In a specific embodiment of this portion of the invention, a HIV-1 DNA pol construct contains a mutation (or mutations) within the Pol coding region which effectively abolishes RT, RNase H and IN activity. A specific HIV-1 pol-containing construct contains at least one point mutation which alters the active site of the RT, RNase H and IN domains of Pol, such that each activity is at least substantially abolished. Such a HIV-1 Pol mutant will most likely comprise at least one point mutation in or around each catalytic domain responsible for RT, RNase H and IN activity, respectfully. To this end, specific embodiments relate to adenoviral vectors comprising HIV-1 pol wherein the encoding nucleic acid comprises nine codon substitution mutations which result in an inactivated Pol protein (IA Pol: SEQ ID NO: 8, FIGS. 11A-1 to 11A-3) which has no PR, RT, RNase or IN activity, wherein three such point mutations reside within each of the RT, RNase and IN catalytic domains. Therefore, one exemplification contemplated employs an adenoviral vector construct which comprises, in an appropriate fashion, a nucleic acid molecule which encodes IA-Pol, which contains all nine mutations as shown below in Table 1. An additional amino acid residue for substitution is Asp551, localized within the RNase domain of Pol. Any combination of the mutations disclosed herein may be suitable and therefore may be utilized in the vectors, methods and compositions of the present invention. While addition and deletion mutations are contemplated and within the scope of the invention, the preferred mutation is a point mutation resulting in a substitution of the wild type amino acid with an alternative amino acid residue.

TABLE 1

| wt aa | aa residue | mutant aa | enzyme function |
|---|---|---|---|
| Asp | 112 | Ala | RT |
| Asp | 187 | Ala | RT |
| Asp | 188 | Ala | RT |
| Asp | 445 | Ala | RNase H |
| Glu | 480 | Ala | RNase H |
| Asp | 500 | Ala | RNase H |
| Asp | 626 | Ala | IN |
| Asp | 678 | Ala | IN |
| Glu | 714 | Ala | IN |

It is preferred that point mutations be incorporated into the IApol mutant adenoviral vector constructs of the present invention so as to lessen the possibility of altering epitopes in and around the active site(s) of HIV-1 Pol. To this end, SEQ ID NO: 8 (FIGS. 11A-1 to 11A-3) discloses the nucleotide sequence which codes for a codon optimized pol in addition to the nine mutations shown in Table 1 and referred to herein as “IApol”.

In one specific example of an adenoviral vector comprising pol (see Table 1), all residues that comprise the catalytic triad of the polymerase, namely Asp112, Asp187, and Asp 188, can be substituted with alanine (Ala) residues (Larder, et al., Nature 1987, 327: 716-717; Larder, et al., 1989, Proc. Natl. Acad. Sci. 1989, 86: 4803-4807). Three additional mutations can be introduced at Asp445, Glu480 and Asp500 to abolish RNase H activity, with each residue being substituted for an Ala residue, respectively (Davies, et al., 1991, Science 252, 88-95; Schatz, et al., 1989, FEBS Lett. 257: 311-314; Mizrahi, et al., 1990, Nucl. Acids. Res. 18: pp. 5359-5353). HIV pol integrase function can be abolished through three mutations at Asp626, Asp678 and Glu714. Again, each of these residues can be substituted with an Ala residue (Wiskerchen, et al., 1995, J. Virol. 69: 376-386; Leavitt, et al., 1993, J. Biol. Chem. 268: 2113-2119). Amino acid residue Pro3 of SEQ ID NO: 8 marks the start of the RT gene. The complete amino acid sequence of IA-Pol is disclosed herein as SEQ ID NO: 8 and shown in FIGS. 11A-1 to 11A-3.

It will be understood that any combination of mutations may be suitable and therefore utilized in the adenoviral HIV constructs, methods and compositions disclosed herein either administered alone, with other heterologous genes, in a combined modality regime and/or as part of a prime-boost regimen. For example, it may be possible to mutate only 2 of the 3 residues within the respective reverse transcriptase, RNase H, and integrase coding regions while still abolishing these enzymatic activities.

Another feature of the present invention are methods, vectors and compositions employing adenoviral vector constructs comprising codon optimized HIV-1 Pol comprising a eukaryotic trafficking signal peptide or a leader peptide such as that found in highly expressed mammalian proteins such as immunoglobulin leader peptides. Any functional leader peptide may be tested for efficacy. The respective DNA may be modified by known recombinant DNA methodology. In the alternative, as noted above, a nucleotide sequence which encodes a leader/signal peptide may be inserted into a DNA vector housing the open reading frame for the Pol protein of interest. Regardless of the cloning strategy, the end result is a vector construct which comprises vector components for effective gene expression in conjunction with nucleotide sequences which encode a modified HIV-1 Pol protein of interest, including but not limited to a HIV-1 Pol protein which contains a leader peptide.

The design of gene sequences disclosed herein incorporates the use of human preferred (“humanized”) codons for each amino acid residue in the sequence in order to maximize in vivo mammalian expression (Lathe, 1985, J. Mol. Biol. 183:1-12). As can be discerned by inspecting the codon usage in SEQ ID NOs: 6 and 8, the following codon usage for mammalian optimization is preferred: Met (ATG), Gly (GGC), Lys (AAG), Trp (TGG), Ser (TCC), Arg (AGG), Val (GTG), Pro (CCC), Thr (ACC), Glu (GAG); Leu (CTG), His (CAC), Ile (ATC), Asn (AAC), Cys (TGC), Ala (GCC), Gln (CAG), Phe (TTC) and Tyr (TAC). For an additional discussion relating to mammalian (human) codon optimization, see WO 97/31115 (PCT/US97/02294). It is intended that the skilled artisan may use alternative versions of codon optimization or may omit this step when generating HIV vaccine constructs within the scope of the present invention. Therefore, the present invention also relates to vectors, methods and compositions comprising/utilizing non-codon optimized or partially codon optimized versions of nucleic acid molecules and associated recombinant adenoviral HIV constructs which encode the various wild type and modified forms of the HIV proteins. However, codon optimization of these constructs constitutes a preferred embodiment of this invention.

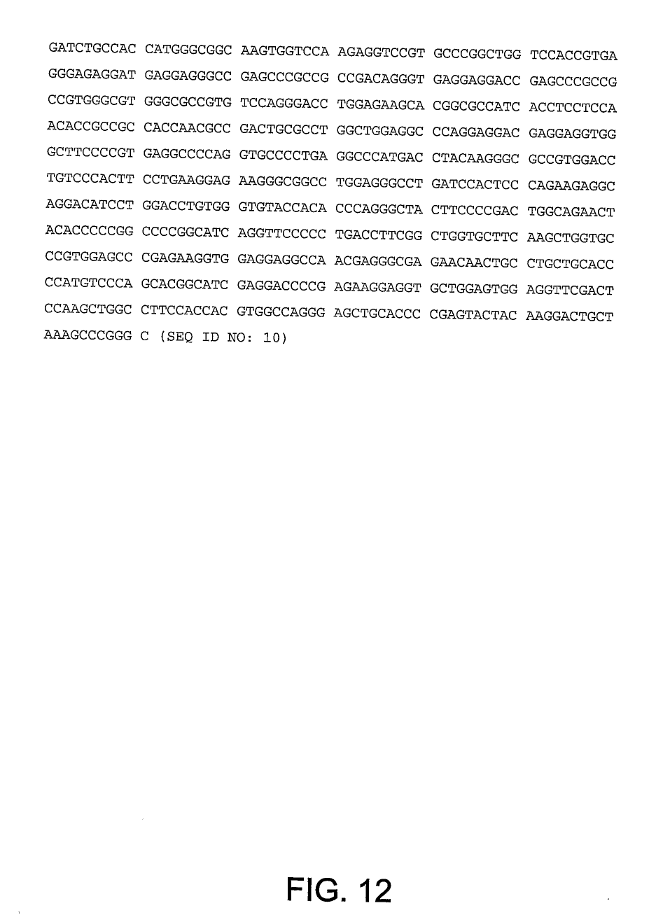

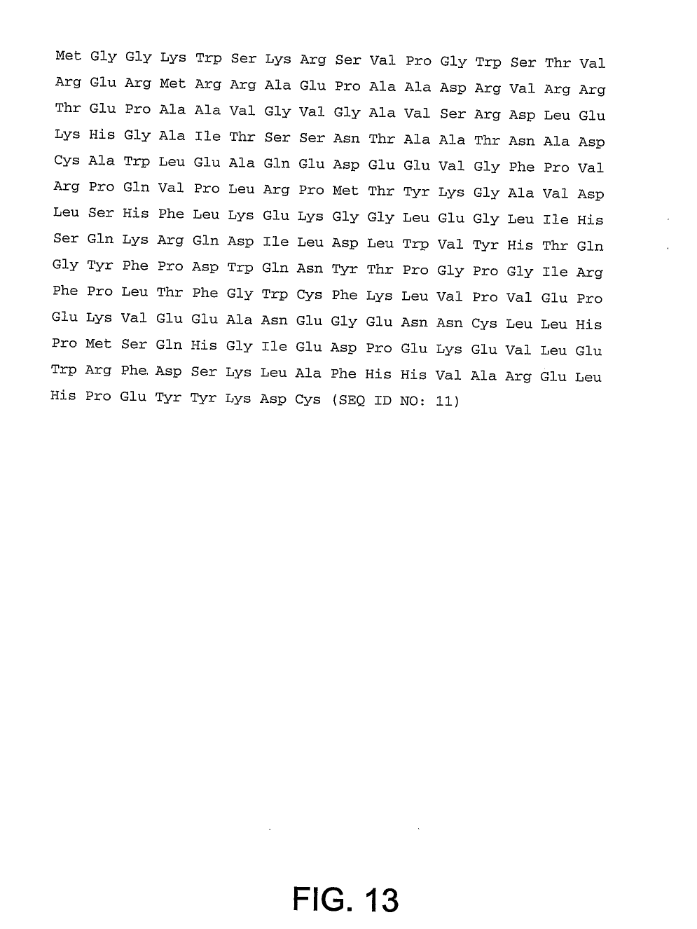

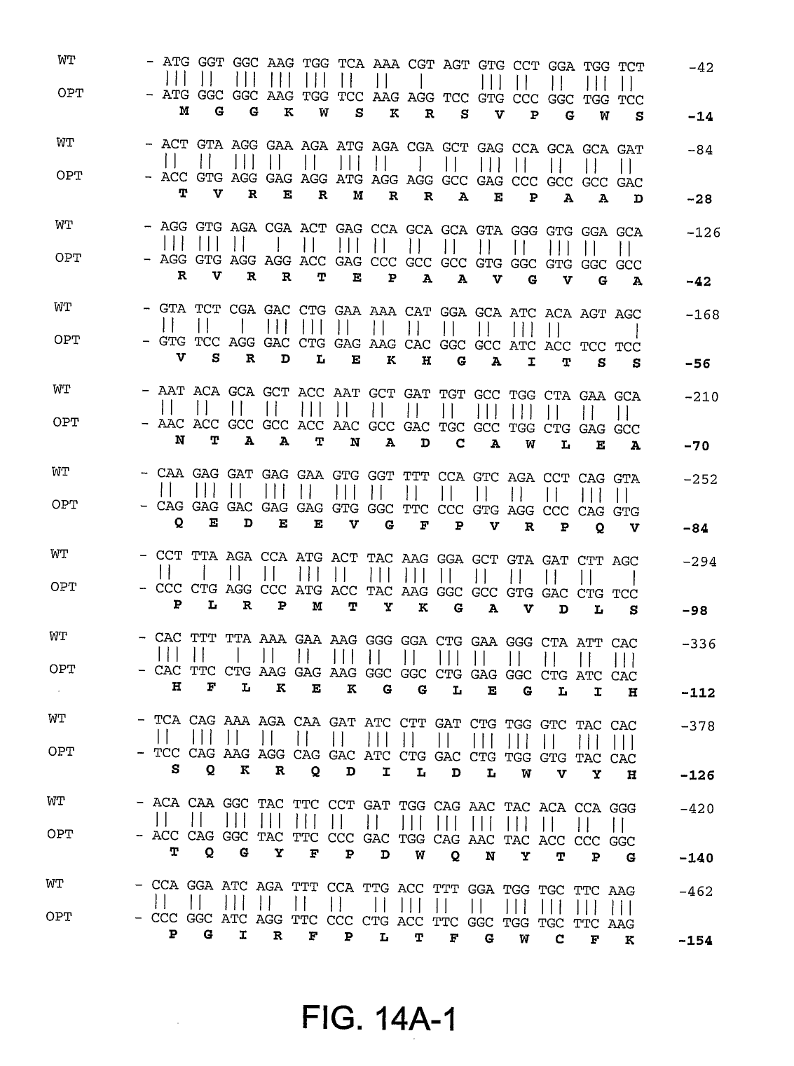

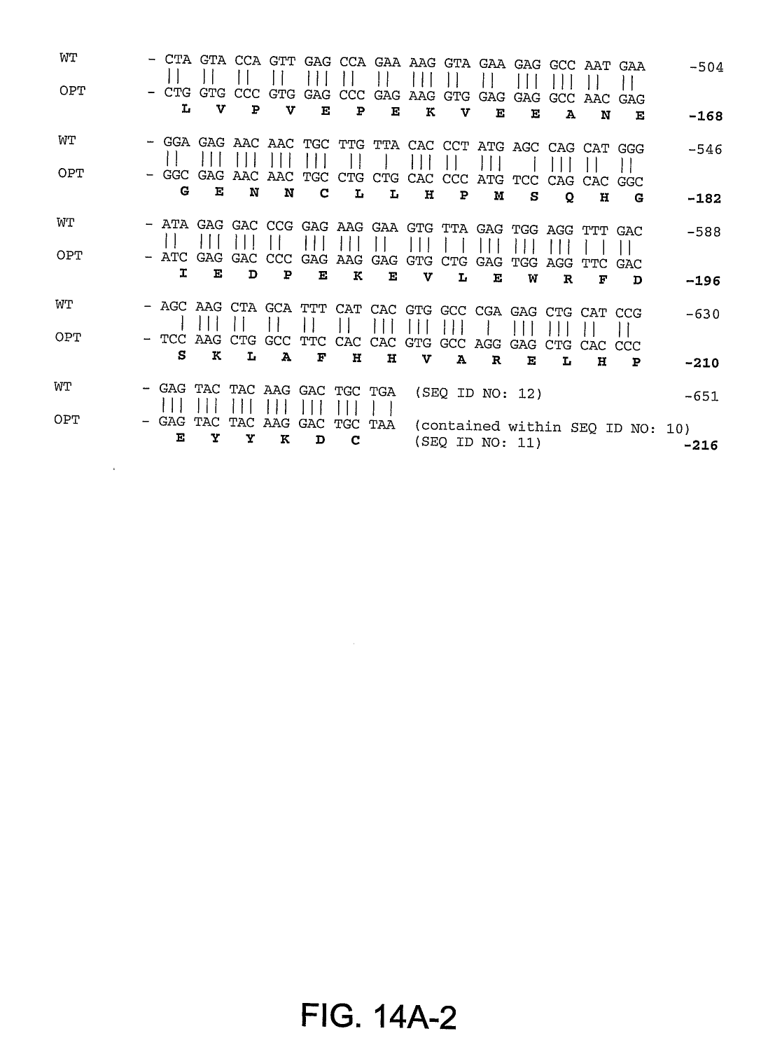

Codon optimized versions of HIV-1 nef and HIV-1 nef modifications of use in specific embodiments herein can be found in U.S. application Ser. No. 09/738,782, filed Dec. 15, 2000 and PCT International Application PCT/US00/34162, also filed Dec. 15, 2000. Specific codon optimized nef and nef modifications relate to nucleic acid encoding HIV-1 Nef from the HIV-1 jrfl isolate wherein the codons are optimized for expression in a mammalian system such as a human. A DNA molecule which encodes this protein is disclosed herein as SEQ ID NO: 10 (FIG. 12), while the expressed open reading frame is disclosed herein as SEQ ID NO: 11. FIGS. 14A-1 to 14A-2 illustrate a comparison of wild type vs. codon optimized nucleotides comprising the open reading frame of HIV-nef. The open reading frame for SEQ ID NO: 10 comprises an initiating methionine residue at nucleotides 12-14 and a “TAA” stop codon from nucleotides 660-662. The open reading frame of SEQ ID NO: 10 provides a 216 amino acid HIV-1 Nef protein expressed through utilization of a codon optimized DNA vaccine vector. The 216 amino acid HIV-1 Nef (jrfl) protein is disclosed herein as SEQ ID NO: 11; FIG. 13. A modified nef optimized coding region forming an additional embodiment herein relates to a nucleic acid molecule encoding optimized HIV-1 Nef wherein the open reading frame codes for modifications at the amino terminal myristylation site (Gly-2 to Ala-2) and substitution of the Leu-174-Leu-175 dileucine motif to Ala-174-Ala-175, herein described as opt nef (G2A, LLAA). A DNA molecule which encodes this protein is disclosed herein as SEQ ID NO: 13, while the expressed open reading frame is disclosed herein as SEQ ID NO: 14. Yet another modified nef optimized coding region forming an embodiment hereof relates to a nucleic acid molecule encoding optimized HIV-1 Nef wherein the open reading frame codes for modifications at the amino terminal myristylation site (Gly-2 to Ala-2), herein described as opt nef (G2A). A DNA molecule which encodes this protein is disclosed herein as SEQ ID NO: 15, while the expressed open reading frame is disclosed herein as SEQ ID NO: 16.

HIV-1 Nef is a 216 amino acid cytosolic protein which associates with the inner surface of the host cell plasma membrane through myristylation of Gly-2 (Franchini et al., 1986, Virology 155: 593-599). While not all possible Nef functions have been elucidated, it has become clear that correct trafficking of Nef to the inner plasma membrane promotes viral replication by altering the host intracellular environment to facilitate the early phase of the HIV-1 life cycle and by increasing the infectivity of progeny viral particles. In one aspect of the invention, the methods, vectors and compositions of the present invention employ an adenoviral vector(s) comprising codon-optimized nef sequence modified to contain a nucleotide sequence which encodes a heterologous leader peptide such that the amino terminal region of the expressed protein will contain the leader peptide. The diversity of function that typifies eukaryotic cells depends upon the structural differentiation of their membrane boundaries. To generate and maintain these structures, proteins must be transported from their site of synthesis in the endoplasmic reticulum to predetermined destinations throughout the cell. This requires that the trafficking proteins display sorting signals that are recognized by the molecular machinery responsible for route selection located at the access points to the main trafficking pathways. Sorting decisions for most proteins need to be made only once as they traverse their biosynthetic pathways since their final destination, the cellular location at which they perform their function, becomes their permanent residence. Maintenance of intracellular integrity depends in part on the selective sorting and accurate transport of proteins to their correct destinations. Defined sequence motifs exist in proteins which can act as ‘address labels’. A number of sorting signals have been found associated with the cytoplasmic domains of membrane proteins. An effective induction of CTL responses often requires sustained, high level endogenous expression of an antigen. As membrane-association via myristylation is an essential requirement for most of Nef's function, mutants lacking myristylation, by glycine-to-alanine change, change of the dileucine motif and/or by substitution with a leader sequence, will be functionally defective, and therefore will have improved safety profile compared to wild-type Nef for use as an HIV-1 vaccine component.

In specific embodiments, therefore, the nucleotide sequence is modified to include a leader or signal peptide of interest. This may be accomplished by known recombinant DNA methodology. In the alternative, as noted above, insertion of a nucleotide sequence may be inserted into a DNA vector housing the open reading frame for the Nef protein of interest.

It has been shown that myristylation of Gly-2 in conjunction with a dileucine motif in the carboxy region of the protein is essential for Nef-induced down regulation of CD4 (Aiken et al., 1994, Cell 76: 853-864) via endocytosis. It has also been shown that Nef expression promotes down regulation of MHCI (Schwartz et al., 1996, Nature Medicine 2(3): 338-342) via endocytosis. The present invention contemplates adenoviral vectors which comprise sequence encoding a modified Nef protein altered in trafficking and/or functional properties and the use thereof in the methods and compositions of the present invention. The modifications introduced into the adenoviral vector HIV constructs of the present invention include but are not limited to additions, deletions or substitutions to the nef open reading frame which results in the expression of a modified Nef protein which includes an amino terminal leader peptide, modification or deletion of the amino terminal myristylation site, and modification or deletion of the dileucine motif within the Nef protein and which alter function within the infected host cell.

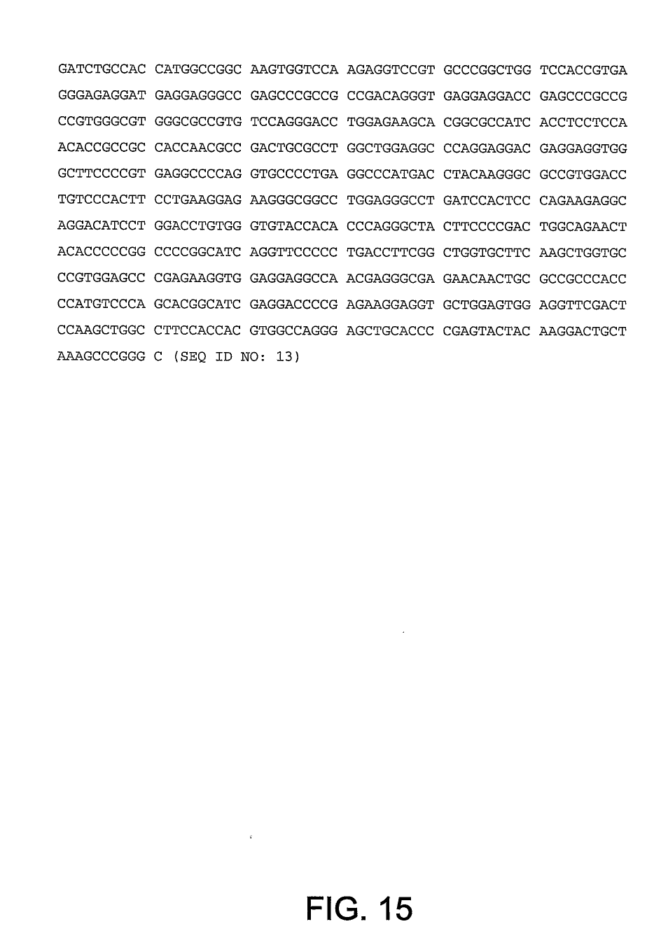

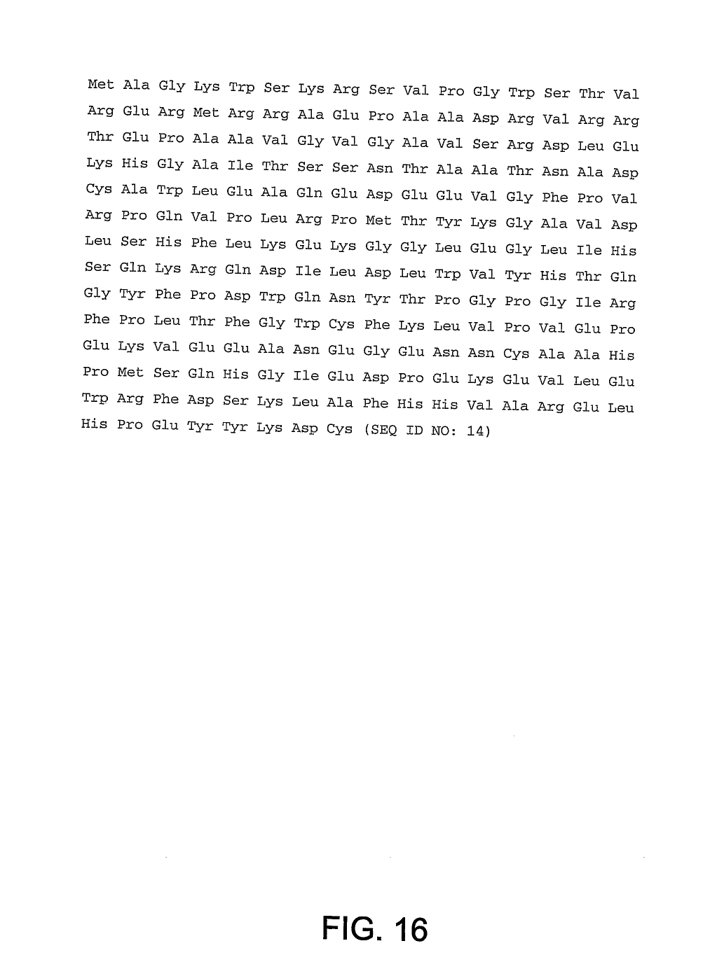

A recombinant adenoviral construct of use in accordance with the methods and compositions disclosed herein can comprise sequence encoding optimized HIV-1 Nef with modifications at the amino terminal myristylation site (Gly-2 to Ala-2) and substitution of the Leu-174-Leu-175 dileucine motif to Ala-174-Ala-175. This open reading frame is herein described as opt nef (G2A,LLAA) and is disclosed as SEQ ID NO: 13, which comprises an initiating methionine residue at nucleotides 12-14 and a “TAA” stop codon from nucleotides 660-662. The nucleotide sequence of this codon optimized version of HIV-1 jrfl nef gene with the above mentioned modifications is disclosed herein as SEQ ID NO: 13; FIG. 15. The open reading frame of SEQ ID NO: 13 encodes Nef (G2A,LLAA), disclosed herein as SEQ ID NO: 14; FIG. 16.

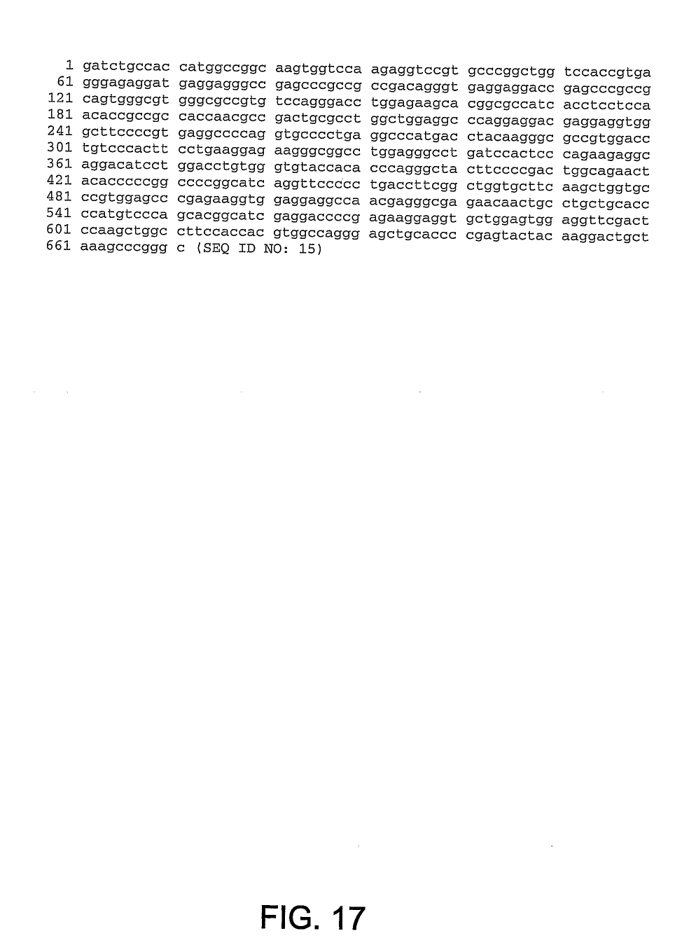

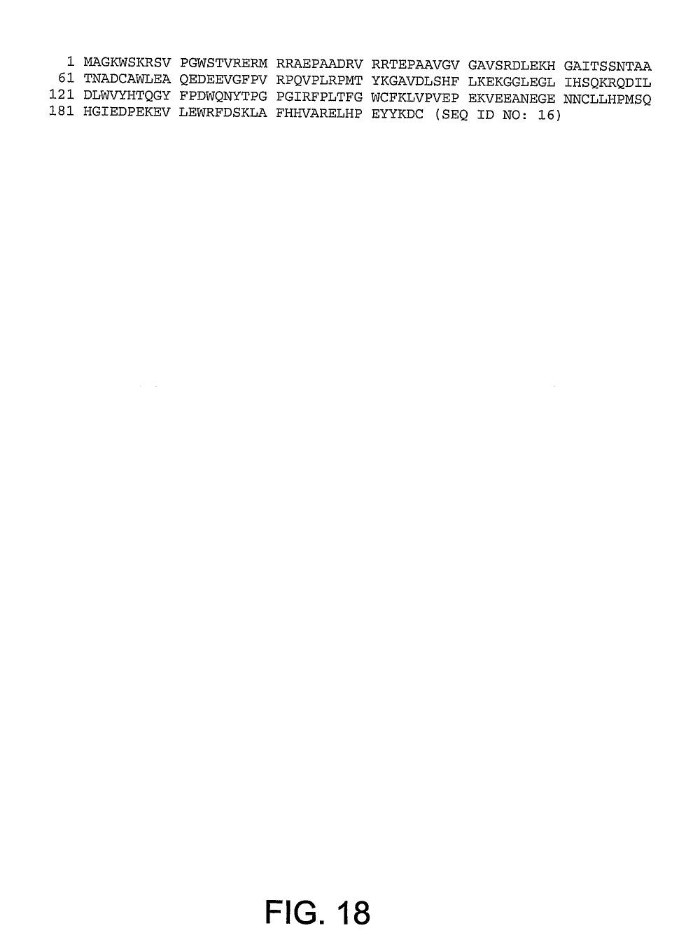

Another recombinant adenoviral construct of use in accordance with the methods and compositions disclosed herein can comprise sequence encoding optimized HIV-1 Nef with modifications at the amino terminal myristylation site (Gly-2 to Ala-2). This open reading frame is herein described as opt nef (G2A) and is disclosed as SEQ ID NO: 16, which comprises an initiating methionine residue at nucleotides 12-14 and a “TAA” stop codon from nucleotides 660-662. The nucleotide sequence of this codon optimized version of HIV-1 jrfl nef gene with the above mentioned modification is disclosed herein as SEQ ID NO: 15; FIG. 17. The open reading frame of SEQ ID NO: 15 encodes Nef (G2A), disclosed herein as SEQ ID NO: 16; FIG. 18.

FIG. 19 shows a schematic presentation of nef and nef derivatives. Amino acid residues involved in Nef derivatives are presented. Glycine 2 and Leucine 174 and 175 are the sites involved in myristylation and dileucine motif, respectively.

Adenoviral vectors of use in the methods and compositions of the present invention may comprise one or more HIV genes/encoding nucleic acid. The administration of at least one recombinant adenoviral vector(s) comprising two or more HIV genes, their derivatives, or modifications are anticipated as well as exemplified herein. Two or more HIV genes can be expressed on at least one recombinant adenoviral vector construct and/or two or more HIV genes can be expressed across two or more constructs. Therefore, the present invention offers the possibility of using the methods and compositions of the present invention to evade/bypass host immunity and effectuate a multi-valent HIV gene administration, specific examples, but not limitations of which, include the administration of adenoviral vectors comprising nucleic acid sequence encoding (1) Gag and Nef polypeptides, (2) Gag and Pol polypeptides, (3) Pol and Nef polypeptides, and (4) Gag, Pol and Nef polypeptides.

Multiple genes/encoding nucleic acid may be ligated into a proper shuttle plasmid for generation of a pre-adenoviral plasmid comprising multiple open reading frames. Open reading frames for the multiple genes/encoding nucleic acid can be operatively linked to distinct promoters and transcription termination sequences. In other embodiments, the open reading frames may be operatively linked to a single promoter, with the open reading frames operatively linked by an internal ribosome entry sequence (IRES; as disclosed in WO 95/24485), or suitable alternative allowing for transcription of the multiple open reading frames to run off of a single promoter. In certain embodiments, the open reading frames may be fused together by stepwise PCR or suitable alternative methodology for fusing together two open reading frames. Various combined modality administration regimens suitable for use in the present invention are disclosed in PCT/US01/28861, published Mar. 21, 2002.

Multi-valent vectors of this description form an important aspect of the present invention as are methods of using same in eliciting cellular-mediated immune responses specific for the HIV antigens contained therein. It is well within the purview of one of skill in the art to arrive at and effectively utilize various fusion/multi-valent constructs.

The present invention encompasses methods for (1) effectuating a therapeutic response in an individual and (2) inducing an immune response (including a cellular-mediated immune response) comprising administering to an individual a recombinant adenovirus serotype 26 vector in accordance with the present invention. One aspect of the present invention are methods for generating an enhanced immune response against one or more antigens (bacterial, viral (e.g., HIV) or other (e.g., cancer)) which comprise the administration of a recombinant adenovirus serotype 26 vehicle expressing the antigen of interest. Administration of recombinant Ad26 vectors in this manner provides for improved cellular-mediated immune response, particularly where there is pre-existing immunity in a given host to the more well-represented adenovirus serotypes (e.g., Ad2 and Ad5). An effect of the improved vaccine administration methods should be a lower transmission rate to (or occurrence rate in) previously uninfected individuals (i.e., prophylactic applications) and/or a reduction in the levels of virus/bacteria/foreign agent within an infected individual (i.e., therapeutic applications). As relates to HIV indications, an effect of the improved vaccine administration methods should be a lower transmission rate to previously uninfected individuals (i.e., prophylactic applications) and/or a reduction in the levels of viral loads within an infected individual (i.e., therapeutic applications) so as to prolong the asymptomatic phase of HIV infection. Administration, intracellular delivery and expression of the recombinant Ad26 vectors elicits a host CTL and Th response.

Accordingly, the present invention relates to methodology regarding administration of the recombinant Ad26 viral vectors (or immunogenic compositions thereof, herein termed vaccines) to provide effective immunoprophylaxis, to prevent establishment of an infection following exposure to the viral (for instance, HIV), bacterial or other agent, or as a post-infection therapeutic vaccine to mitigate infection to result in the establishment of a lower virus/bacteria/other load with beneficial long term consequences.

The recombinant adenovirus serotype 26 vectors disclosed herein may form the subject of a single administration or be part of a broader prime/boost-type administration regimen. Prime-boost regimens can employ different viruses (including but not limited to different viral serotypes and viruses of different origin), viral vector/protein combinations, and combinations of viral and polynucleotide administrations. In this type of scenario, an individual is first administered a priming dose of a protein/antigen/derivative/modification utilizing a certain vehicle (be that a viral vehicle, purified and/or recombinant protein, or encoding nucleic acid). Multiple primings, typically 1-4, are usually employed, although more may be used. The priming dose(s) effectively primes the immune response so that, upon subsequent identification of the protein/antigen(s) in the circulating immune system, the immune response is capable of immediately recognizing and responding to the protein/antigen(s) within the host. Following some period of time, the individual is administered a boosting dose of at least one of the previously delivered protein(s)/antigen(s), derivatives or modifications thereof (administered by viral vehicle/protein/nucleic acid). The length of time between priming and boost may typically vary from about four months to a year, albeit other time frames may be used as one of ordinary skill in the art will appreciate. The follow-up or boosting administration may also be repeated at selected time intervals. A mixed modality prime and boost inoculation scheme should result in an enhanced immune response, specifically where there is pre-existing anti-vector immunity.

Selection of the alternate administration vehicle (be it viral/nucleic acid/protein) to be employed in conjunction with the vectors disclosed herein in a prime-boost administration regimen is not critical to the successful practice hereof. Any vehicle capable of delivering the antigen (or effectuating expression of the antigen) to sufficient levels such that a cellular and/or humoral-mediated response is elicited should be sufficient to prime or boost the presently disclosed administration. Suitable viral vehicles include but are not limited to distinct serotypes of adenovirus, including but not limited to adenovirus serotypes 5, 6, 24, 34 and 35 (see, e.g., PCT/US00/18332, published Jan. 11, 2001 (Ad5); PCT/US01/28861, published Mar. 21, 2002 (Ad5); PCT/US02/32512, published Apr. 17, 2003 (Ad6); PCT/US2003/026145, published Mar. 4, 2004 (Ad24, Ad34); PCT/NL00/00325, published Nov. 23, 2000 (Ad35)). Alternatively, the adenoviral administration can be followed or preceded by a viral vehicle of diverse origin. Examples of different viral vehicles include but are not limited to adeno-associated virus (“AAV”; see, e.g., Samulski et al., 1987 J. Virol. 61:3096-3101; Samulski et al., 1989 J. Virol. 63:3822-3828); retrovirus (see, e.g., Miller, 1990 Human Gene Ther. 1:5-14; Ausubel et al., Current Protocols in Molecular Biology); pox virus (including but not limited to replication-impaired NYVAC, ALVAC, TROVAC and MVA vectors, see, e.g., Panicali & Paoletti, 1982 Proc. Natl. Acad. Sci. USA 79:4927-31; Nakano et al. 1982 Proc. Natl. Acad. Sci. USA 79: 1593-1596; Piccini et al., In Methods in Enzymology 153:545-63 (Wu & Grossman, eds., Academic Press, San Diego); Sutter et al., 1994 Vaccine 12:1032-40; Wyatt et al., 1996 Vaccine 15:1451-8; and U.S. Pat. Nos. 4,603,112; 4,769,330; 4,722,848; 4,603,112; 5,110,587; 5,174,993; and 5,185,146); and alpha virus (see, e.g., WO 92/10578; WO 94/21792; WO 95/07994; and U.S. Pat. Nos. 5,091,309 and 5,217,879). Prime-boost protocols exploiting adenoviral and pox viral vectors for delivery of HIV antigens are discussed in International Application No. PCT/US03/07511, published Sep. 18, 2003. An alternative to the above immunization schemes would be to employ polynucleotide administrations (including but not limited to “naked DNA” or facilitated polynucleotide delivery) in conjunction with an adenoviral prime and/or boost; see, e.g., Wolff et al., 1990 Science 247:1465, and the following patent publications: U.S. Pat. Nos. 5,580,859; 5,589,466; 5,739,118; 5,736,524; 5,679,647; WO 90/11092 and WO 98/04720. Another alternative would be to employ purified/recombinant protein administration in a prime-boost scheme along with adenovirus.

Potential hosts/vaccinees/individuals that can be administered the recombinant adenoviral vectors of the present invention include, but are not limited to, primates and especially humans and non-human primates, and include any non-human mammal of commercial or domestic veterinary importance.

Compositions of adenoviral vectors including, but not limited to, vaccine compositions, administered in accordance with the methods and compositions of the present invention may be administered alone or in combination with other viral- or non-viral-based DNA/protein vaccines. They also may be administered as part of a broader treatment regimen. The present invention, thus, encompasses those situations where the disclosed adenovirus constructs are administered in conjunction with other therapies; including but not limited to other antimicrobial (e.g., antiviral, antibacterial) agent treatment therapies. Any specific antimicrobial agent(s) is not critical to successful practice of the methods disclosed herein. The antimicrobial agent can, for example, be based on/derived from an antibody, a polynucleotide, a polypeptide, a peptide, or a small molecule. Any antimicrobial agent that effectively reduces microbial replication/spread/load within an individual is sufficient for the uses described herein.

Antiviral agents antagonize the functioning/life cycle of a virus, and target a protein/function essential to the proper life cycle of the virus; an effect that can be readily determined by an in vivo or in vitro assay. Some representative antiviral agents which target specific viral proteins are protease inhibitors, reverse transcriptase inhibitors (including nucleoside analogs; non-nucleoside reverse transcriptase inhibitors; and nucleotide analogs), and integrase inhibitors. Protease inhibitors include, for example, indinavir/CRIXIVAN®; ritonavir/NORVIR®; saquinavir/FORTOVASE®; nelfinavir/VIRACEPT®; amprenavir/AGENERASE®; lopinavir and ritonavir/KALETRA®. Reverse transcriptase inhibitors include, for example, (1) nucleoside analogs, e.g., zidovudine/RETROVIR® (AZT); didanosine/VIDEX® (ddI); zalcitabine/HIVID® (ddC); stavudine/ZERIT® (d4T); lamivudine/EPIVIR® (3TC); abacavir/ZIAGEN® (ABC); (2) non-nucleoside reverse transcriptase inhibitors, e.g., nevirapine/VIRAMUNE® (NVP); delavirdine/RESCRIPTOR® (DLV); efavirenz/SUSTIVA® (EFV); and (3) nucleotide analogs, e.g., tenofovir DF/VIREAD® (TDF). Integrase inhibitors include, for example, the molecules disclosed in U.S. Application Publication No. US2003/0055071, published Mar. 20, 2003; and International Application WO 03/035077. The antiviral agents, as indicated, can target as well a function of the virus/viral proteins, such as, for instance the interaction of regulatory proteins tat or rev with the trans-activation response region (“TAR”) or the rev-responsive element (“RRE”), respectively. An antiviral agent is, preferably, selected from the class of compounds consisting of: a protease inhibitor, an inhibitor of reverse transcriptase, and an integrase inhibitor. Preferably, the antiviral agent administered to an individual is some combination of effective antiviral therapeutics such as that present in highly active anti-retroviral-therapy (“HAART”) a term generally used in the art to refer to a cocktail of inhibitors of viral protease and reverse transcriptase.

One of skill in the art can appreciate that the present invention can be employed in conjunction with any pharmaceutical composition useful for the treatment of microbial infections. Antimicrobial agents are typically administered in their conventional dosage ranges and regimens as reported in the art, including the dosages described in the Physicians' Desk Reference, 54^{th} edition, Medical Economics Company, 2000.

Compositions comprising the recombinant viral vectors may contain physiologically acceptable components including, but not limited to, buffer, normal saline or phosphate buffered saline, sucrose, other salts and polysorbate. In specific embodiments the viral particles are formulated in A195 formulation buffer. In certain embodiments, the formulation has: 2.5-10 mM TRIS buffer, preferably about 5 mM TRIS buffer; 25-100 mM NaCl, preferably about 75 mM NaCl; 2.5-10% sucrose, preferably about 5% sucrose; 0.01-2 mM MgCl₂; and 0.001%-0.01% polysorbate 80 (plant derived). The pH should range from about 7.0-9.0, preferably about 8.0. One skilled in the art will appreciate that other conventional vaccine excipients may also be used in the formulation. In specific embodiments, the formulation contains 5 mM TRIS, 75 mM NaCl, 5% sucrose, 1 mM MgCl₂, 0.005% polysorbate 80 at pH 8.0. This has a pH and divalent cation composition which is near the optimum for virus stability and minimizes the potential for adsorption of virus to glass surface. It does not cause tissue irritation upon intramuscular injection. It is preferably frozen until use.

The amount of viral particles in the vaccine composition(s) to be introduced into a vaccine recipient will depend on the strength of the transcriptional and translational promoters used and on the immunogenicity of the expressed gene product(s). In general, an immunologically or prophylactically effective dose of 1×10⁷ to 1×10¹² particles and preferably about 1×10¹⁰ to 1×10¹¹ particles per adenoviral vector is administered directly into muscle tissue. Subcutaneous injection, intradermal introduction, impression through the skin, and other modes of administration such as intraperitoneal, intravenous, or inhalation delivery are also contemplated.

Administration of additional agents able to potentiate or broaden the immune response (e.g., the various cytokines, interleukins), concurrently with or subsequent to parenteral introduction of the viral vectors of this invention, is appreciated herein as well and can be advantageous.

The benefits of administration as described herein should be (1) a comparable or broader population of individuals successfully immunized/treated with recombinant adenoviral vectors, and (2) in situations of immunization, a lower transmission rate to (or occurrence rate in) previously uninfected individuals (i.e., prophylactic applications) and/or a reduction in/control of the levels of virus/bacteria/foreign agent within an infected individual (i.e., therapeutic applications).

The following non-limiting Examples are presented to better illustrate the workings of the invention.

### EXAMPLE 1

### Construction of pAd26ΔE1ΔE4Ad5Orf6

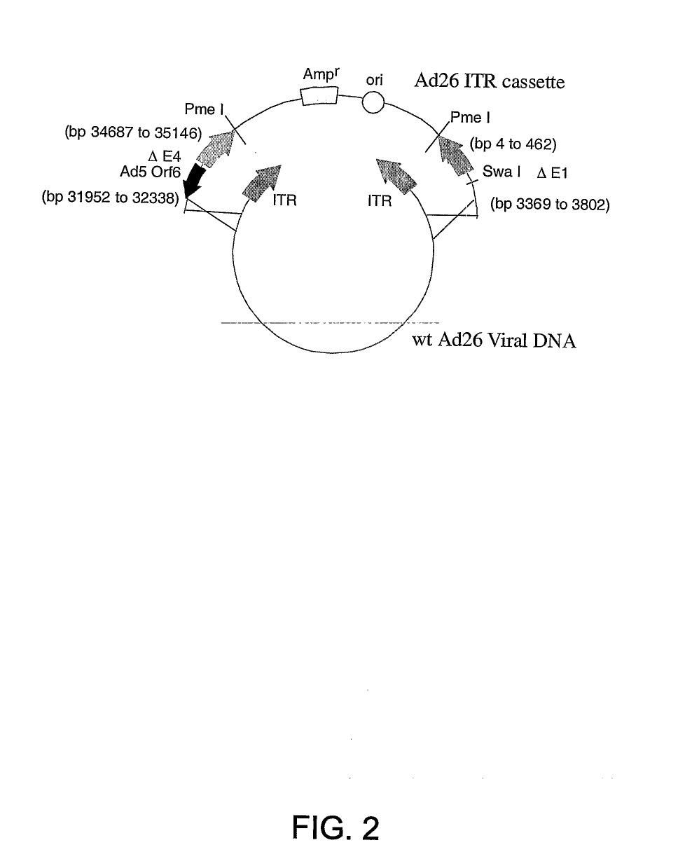

To construct pAd26ΔE1ΔE4Ad5Orf6 (An Ad26 pre-Ad plasmid containing an E1 deletion and an E4 deletion substituted with Ad5 Orf6 in order to enable efficient propagation in existing group C/Ad5 E1 complementing cell lines), an Ad26 ITR cassette was constructed containing sequences from the right (bp 31952 to 32338 and bp 34687 to 35146) and left (bp 4 to 462 and bp 3369 to 3802) end of the Ad26 genome (see FIGS. 1A-1 to 1A-11) separated by plasmid sequences containing a bacterial origin of replication and an ampicillin resistance gene. The four segments were generated by PCR and cloned sequentially into pNEB193, generating pNEBAd26-4a. Next the Ad5 Orf6 open reading frame was generated by PCR and cloned between Ad26 bp 32338 and 34687 generating pNEBAd26-4aAdSOrf6 (the ITR cassette). PNEB 193 is a commonly used commercially available cloning plasmid (New England Biolabs cat# N3051S) containing a bacterial origin of replication, ampicillin resistance gene and a multiple cloning site into which the PCR products were introduced. The ITR cassette contains a deletion of E1 sequences from Ad26 bp 463 to 3368 with a unique Swa I restriction site located in the deletion and an E4 deletion from Ad26 bp 32339 to 34686 into which Ad5 Orf6 was introduced in an E4 parallel orientation. In this construct Ad5Orf6 expression is driven by the Ad26 E4 promoter. The Ad26 sequences (bp 31952 to 32338 and bp 3369 to 3802) in the ITR cassette provided regions of homology with the purified Ad26 viral DNA in which bacterial recombination could occur following cotransformation into BJ 5183 bacteria (FIG. 2). The ITR cassette was also designed to contain unique restriction enzyme sites (PmeI) located at the end of the viral ITR's so that digestion would release the recombinant Ad26 genome from the plasmid sequences. Potential clones were screened by restriction analysis and one clone was selected as pAd26ΔE1ΔE4Ad5Orf6. Pre-Adenovirus plasmid pAd26ΔE1ΔE4Ad5Orf6 contains Ad26 sequences from bp 4 to 462; bp 3369 to bp 32338 and bp 34687 to bp 35146 with Ad5 Orf6 cloned between bp 32338 and bp 34686. This plasmid was completely sequenced (see FIGS. 3A-1 to 3A-9). The bp numbering in the above description refers to the wt sequence for both Ad26 and Ad5.

### EXAMPLE 2

### Insertion of HIV-1 gag and SEAP Transgenes into pAd26ΔE1ΔE4Ad5Orf6

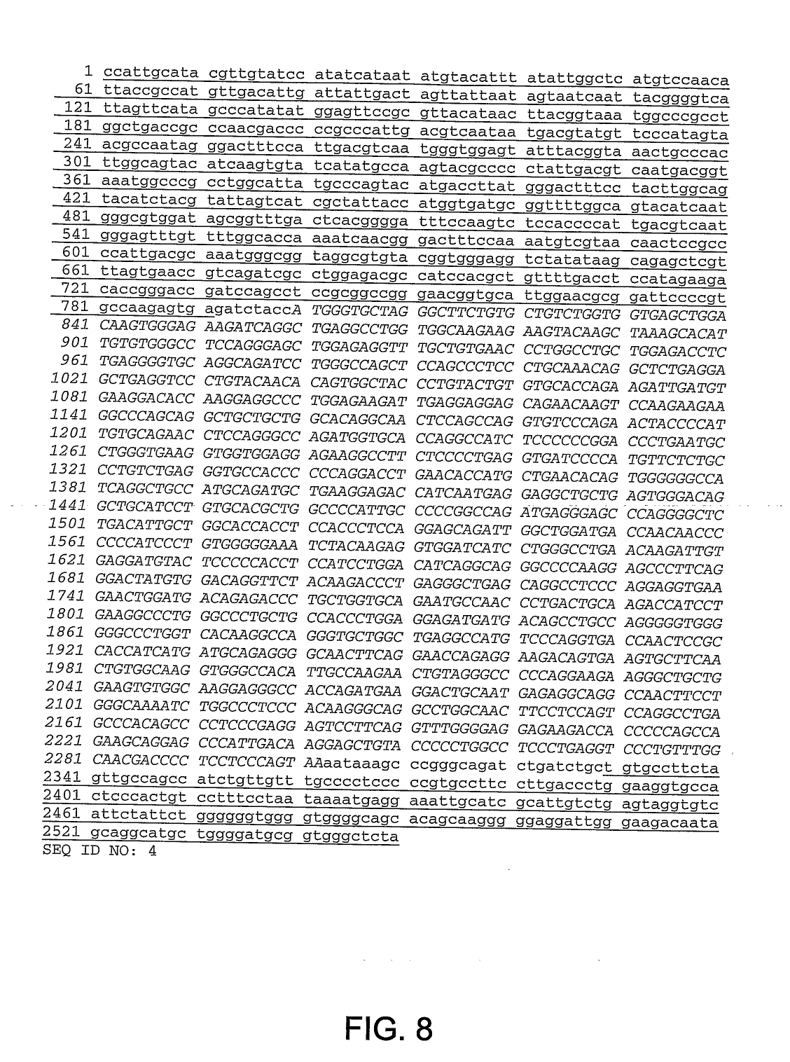

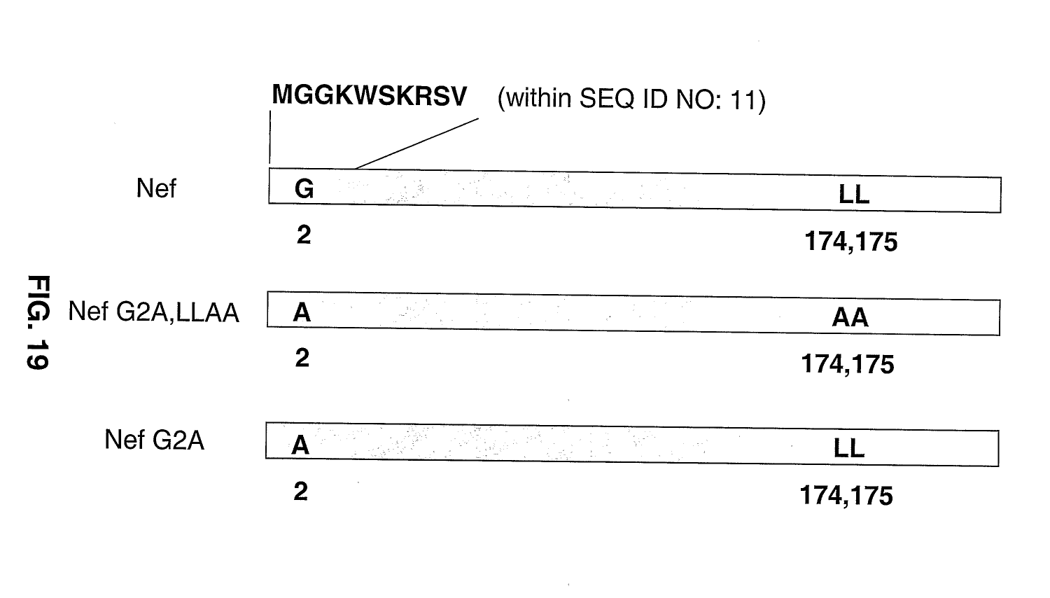

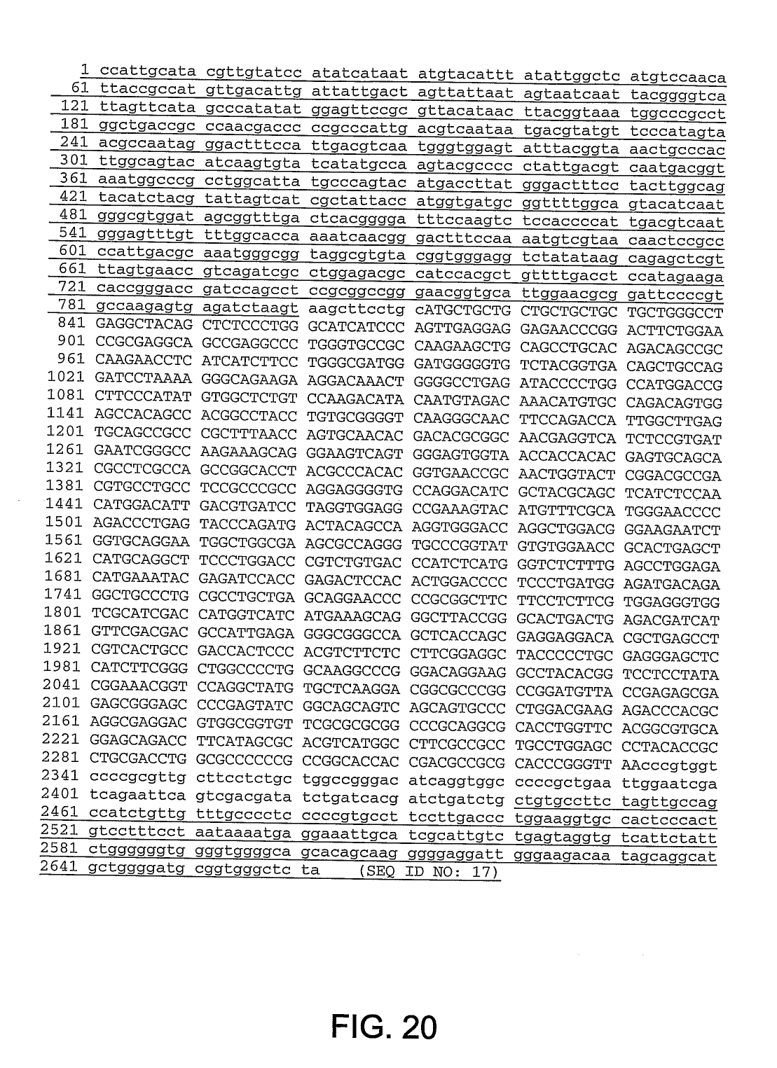

In order to introduce a gag or SEAP expression cassette (see FIGS. 8 and 20, respectively) into the E1 region of pAd26ΔE1ΔE4Ad5Orf6, bacterial recombination was again used. An HIV-1 gag expression cassette consisting of the following: 1) the immediate early gene promoter from human cytomegalovirus, 2) the coding sequence of the human immunodeficiency virus type 1 (HIV-1) gag (strain CAM-1; 1526 bp) gene, and 3) the bovine growth hormone polyadenylation signal sequence, was cloned into the E1 deletion in Ad26 shuttle plasmid, pNEBAd26-2 (a precursor to the Ad26 ITR cassette described above), generating pNEBAd26CMVgagBGHpA. pNEBAd26-2 contains Ad26 sequences from the left end of the genome (bp 4 to 462 and bp 3369 to 3802) that define the E1 deletion. The gag expression cassette was obtained from a previously constructed plasmid and cloned into the E1 deletion between bp 462 and 3369 in the E1 parallel orientation. The shuttle vector containing the gag transgene was digested to generate a DNA fragment consisting of the gag expression cassette flanked by Ad26 bp 4 to 462 and bp 3369 to 3802 and the fragment was purified after electrophoresis on an agarose gel. Cotransformation of BJ 5183 bacteria with the shuttle vector fragment and pAd26ΔE1ΔE4Ad5Orf6, linearized in the E1 region by digestion with Swa I, resulted in the generation of the Ad26 gag-containing pre-Adenovirus plasmid pAd26ΔE1gagΔE4Ad5Orf6 by homologous recombination. Potential clones were screened by restriction analysis.

A similar strategy was used to generate Ad26 pre-Ad plasmids containing a SEAP expression cassette. In this case a SEAP expression cassette consisting of: 1) the immediate early gene promoter from human cytomegalovirus, 2) the coding sequence of the human placental SEAP gene, and 3) the bovine growth hormone polyadenylation signal sequence was cloned into the E1 deletion in the Ad26 shuttle plasmid, pNEBAd26-2, generating pNEBAd26CMVSEAPBGHpA. The transgene was then recombined into pAd26ΔE1ΔE4Ad5Orf6 as described above for the gag transgene.

### EXAMPLE 3

### Rescue of pAd26ΔE1ΔE4Ad5Orf6 pAd26ΔE1gagΔE4Ad5Orf6 and pAd26ΔE1SEAPΔE4Ad5Orf6 into Virus

In order to rescue pre-adenovirus plasmids pAd26ΔE1ΔE4Ad5Orf6, pAd26ΔE1gagΔE4Ad5Orf6 and pAd26ΔE1SEAPΔE4Ad5Orf6 into virus, the plasmids were each digested with Pme I and transfected into T-25 flasks of PER.C6® cells using the calcium phosphate co-precipitation technique (Cell Phect Transfection Kit, Amersham Pharmacia Biotech Inc). PmeI digestion releases the viral genome from plasmid sequences allowing viral replication to occur after cell entry. When cytopathic effect (CPE) was complete, approximately 7-10 days post transfection, the infected cells and media were harvested, freeze/thawed three times and the cell debris pelleted by centrifugation. The cell lysate was then used to infect T-225 flasks of PER.C6® cells at 80-90% confluence. Once CPE was reached, infected cells and media were harvested, freeze/thawed three times and the cell debris pelleted by centrifugation. Clarified cellysates were then used to infect 2-layer NUNC cell factories of PER.C6® cells. Following complete CPE, the virus was purified by ultracentrifugation on CsCl density gradients. In order to verify the genetic structure of the rescued viruses, viral DNA was extracted using pronase treatment followed by phenol chloroform extraction and ethanol precipitation. Viral DNA was then digested with HindIII and treated with Klenow fragment to end-label the restriction fragments with P33-dATP. The end-labeled restriction fragments were then size-fractionated by gel electrophoresis and visualized by autoradiography. The digestion products were compared with the digestion products of the corresponding pre-Adenovirus plasmid (that had been digested with Pme1/HindIII prior to labeling) from which they were derived. The expected sizes were observed, indicating that the viruses had been successfully rescued.

### EXAMPLE 4

### Construction of pMRKAd26ΔE1ΔE3ΔE4Ad5Orf6

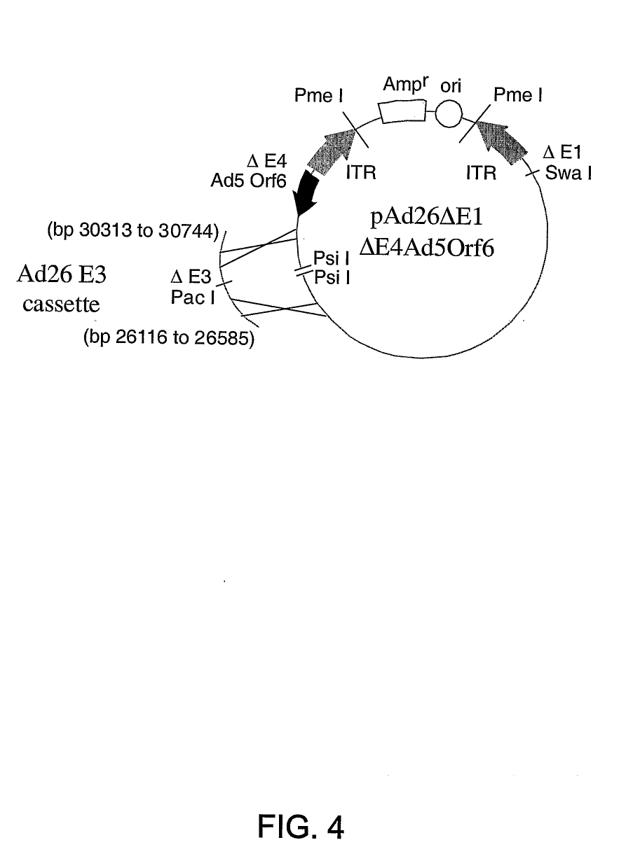

To increase the cloning capacity of our Ad26-based vectors, early region 3 (“E3”) was deleted, generating pre-Ad plasmid pAd26ΔE1ΔE3ΔE4Ad5Orf6. To construct pAd26ΔE1ΔE3ΔE4Ad5Orf6 (an Ad26 pre-Ad plasmid containing an E1 deletion, an E3 deletion and an E4 deletion substituted with Ad5 Orf6), an Ad26 E3 shuttle cassette was constructed containing sequences that defined the desired E3 deletion (bp 26116 to 26585 and bp 30313 to 30744). The two segments were generated by PCR and cloned sequentially into pNEB193, generating pNEBAd26E3-2. The E3 shuttle cassette contains a deletion of E3 sequences from Ad26 bp 26586 and bp 30312 with a unique Pac I restriction site located in the deletion. The E3 shuttle cassette was digested to liberate the Ad26 DNA defining the E3 deletion and the fragment was purified after electrophoresis on an agarose gel. Cotransformation of BJ 5183 bacteria with the shuttle vector fragment and pAd26ΔE1ΔE4Ad5Orf6 (linearized in the E3 region by digestion with Psi I) resulted in the generation of Ad26 pre-Adenovirus plasmid pAd26ΔE1ΔE3ΔE4Ad5Orf6 by homologous recombination (FIG. 4). Potential clones were screened by restriction analysis and one clone was selected as pAd26ΔE1ΔE3ΔE4Ad5Orf6. Pre-Adenovirus plasmid pAd26ΔE1ΔE3ΔE4Ad5Orf6 contains Ad26 sequences from bp 4 to 462; bp 3369 to bp 26585; bp 30313 to 32338; and bp 34687 to bp 35146 with Ad5 Orf6 cloned between bp 32338 and bp 34686. The bp numbering in the above description refers to the wt sequence for both Ad26 and Ad5.

### EXAMPLE 5

### In Vivo Studies

### A. Immunization

Cohorts of three rhesus macaques were immunized with either 10̂8 vp or 10̂10 Vp of Ad26ΔE1gagΔE4Ad5Orf6 (at weeks 0, 4). The animals were given a booster low dose of MRKAd5gag at week 27 (10̂7 vp; pMRKAd5gag disclosed in PCT/US01/28861, published Mar. 21, 2002). A third cohort received priming immunizations with 10̂8 vp MRKAd5 gag followed by a 10̂10 vp booster of Ad26ΔE1gagΔE4Ad5Orf6. Rhesus macaques were between 3-10 kg in weight. In all cases, the total dose of each vaccine was suspended in 1 mL of buffer. The macaques were anesthetized (ketamine/xylazine) and the vaccines were delivered intramuscularly (“i.m.”) in 0.5-mL aliquots into both deltoid muscles using tuberculin syringes (Becton-Dickinson, Franklin Lakes, N.J.). Plasma and peripheral blood mononuclear cells (PBMC) samples were collected following standard protocols.

### B. ELISPOT and ICS Assays

Ninety-six-well flat-bottomed plates (Millipore, Immobilon-P membrane) were coated with 1 μg/well of anti-gamma interferon (IFN-γ) mAb MD-1 (U-Cytech-BV) overnight at 4° C. The plates were then washed three times with PBS and were blocked with RIO medium (RPMI, 0.05 mM 2-mercaptoethanol, 1 mM sodium pyruvate, 2 mM L-glutamate, 10 mM HEPES, 10% fetal bovine serum) for 2 hours at 37° C. The medium was discarded from the plates, and freshly isolated PBMCs were added at 1-4×10⁵ cells/well. The cells were stimulated in the absence (mock) or presence of a gag peptide pool (4 μg/mL per peptide). The pool, which consisted of 15-aa peptides shifted by 4 aas (Synpep, CA), was constructed from the entire HIV-1 CAM1 gag sequence. The cells were then incubated for 20-24 hours at 37° C. in 5% CO₂. Plates were washed six times with PBST (PBS, 0.05% Tween 20) and 100 μL/well of 1:400 dilution of anti-IFN-γ polyclonal biotinylated detector antibody solution (U-Cytech-BV) was added, and the plates were incubated overnight at 37° C. The plates were washed six times with PBST. Color was developed by incubating in NBT/BCP (Pierce) for 10 minutes. Spots were counted under a dissecting microscope and normalized to 1×10⁶ PBMC.

Intracellular staining for IFN-γ production (ICS) was conducted following a previously established protocol; Casimiro et al. 2003 J. Virol. 77:6305-6313.

FIG. 5 lists, in tabular format, the mock-corrected levels of gag-specific T cells as measured by the IFN-γ ELIspot assay.

### C. Results

The Ad26-based vaccine vector was able to effectively prime gag-specific T cells at a dose level of 10̂10 vp. MRKAd5gag-primed animals exhibited responses of greater than 400 SFC/10A6 PBMC after the first dosing. These results indicate that the Ad26 is an effective vector for inducing HIV-specific immunity. For one, the vector is able to prime gag-specific T cells in animals that are boosted significantly using a low effective dose of MRKAd5gag. For group 1, the levels of gag-specific T cells increased 5-20 fold from the pre-boost values. Also, the Ad6 vector itself can be utilized to boost HIV-specific immunity elicited by priming with the MRKAd5gag vector (FIG. 6, group 3).

Analyses of the T cell subsets of the gag-specific immunity by intracellular cytokine staining (ICS) revealed that the Ad26 vector is capable of eliciting both cytotoxic and helper responses.

1. A recombinant adenoviral vector of serotype 26 which is at least partially deleted in E1 and devoid of E1 activity.

2. A recombinant adenoviral vector in accordance with claim 1 which comprises heterologous nucleic acid.

3. A recombinant adenoviral vector in accordance with claim 1 which comprises an E4 gene or a segment of an E4 gene comprising open reading frame 6 (“ORF6”) of an alternative serotype.

4. A recombinant adenoviral vector in accordance with claim 3 wherein the alternative serotype is adenovirus serotype 5.

5. A recombinant adenoviral vector in accordance with claim 2 comprising a gene expression cassette, which comprises:
a) nucleic acid encoding a protein or antigen of interest;
b) a heterologous promoter operatively linked to the nucleic acid of a); and
c) a transcription termination sequence.

6. A recombinant adenoviral vector in accordance with claim 5 wherein the heterologous nucleic acid comprises codons optimized for expression in a human host.

7. A recombinant adenoviral vector in accordance with claim 2 wherein the heterologous nucleic acid encodes an HIV-1 antigen.

8. A recombinant adenoviral vector in accordance with claim 7 wherein the heterologous nucleic acid encodes at least one antigen selected from the group consisting of: HIV-1 Gag, Nef, and Pol.

9. A population of cells comprising the recombinant adenoviral vector of claim 2.

10. A population of cells comprising the recombinant adenoviral vector of claim 3.

11. A method for producing recombinant, replication-defective adenovirus particles comprising:
a) transfecting a recombinant adenoviral vector of claim 2 into a population of cells; and
b) harvesting the resultant recombinant, replication-defective adenovirus.

12. A method for producing recombinant, replication-defective adenovirus particles comprising:
a) transfecting a recombinant adenoviral vector of claim 3 into a population of cells; and
b) harvesting the resultant recombinant, replication-defective adenovirus.

13. Purified recombinant, replication-defective adenovirus particles harvested in accordance with the method of claim 11.

14. Purified recombinant, replication-defective adenovirus particles harvested in accordance with the method of claim 12.

15. A composition comprising purified recombinant adenovirus particles in accordance with claim 13 and a physiologically acceptable carrier.

16. A composition comprising purified recombinant adenovirus particles in accordance with claim 14 and a physiologically acceptable carrier.

17-18. (canceled)

19. A method for delivery and expression of heterologous nucleic acid encoding a protein or antigen of interest, which comprises administering the composition of claim 15 to an individual.

20. A method in accordance with claim 19 wherein administration is preceded or followed by administration of heterologous nucleic acid encoding a protein or antigen of interest with a different vector.

21. A method in accordance with claim 20 wherein the different vector is an adenovirus of a distinct serotype.

22. A method for generating an immune response against an antigen in an individual, which comprises: administering to the individual a composition in accordance with claim 15 wherein the heterologous nucleic acid comprises nucleic acid encoding said antigen.

23. A method in accordance with claim 22 wherein the heterologous nucleic acid encodes at least one antigen selected from the group consisting of: HIV-1 Gag, Nef, and Pol.


SEQUENCE LISTING (17 sequences)

SEQ ID NO: 1 - DNA, 35149 bp, adenovirus serotype 26
catcatcaataatataccccacaaagtaaacaaaagttaatatgcaaatgagcttttgaattttaacggttttggggcggagccaacgctgattggacgagaaacggtgatgcaaatgacgtcacgacgcacggctaacggtcgccgcggaggcgtggcctagcccggaagcaagtcgcggggctgatgacgtataaaaaagcggactttagacccggaaacggccgattttcccgcggccacgcccggatatgaggtaattctgggcggatgcaagtgaaattaggtcattttggcgcgaaaactgaatgaggaagtgaaaagcgaaaaataccggtccctcccagggcggaatatttaccgagggccgagagactttgaccgattacgtgggggtttcgattgcggtgtttttttcgcgaatttccgcgtccgtgtcaaagtccggtgtttatgtcacagatcagctgatccgcagggtatttaaaccagtcgagtccgtcaagaggccactcttgagtgccagcgagtagagatttctctgagctccgctcccagagaccgagaaaaatgagacacctgcgcctcctgccttcaactgtgcccggtgagctggctgtgcttatgctggaggactttgtggatacagtattggaggacgaactgcatccaagtccgttcgagctgggacccacacttcaggatctctatgatctggaggtagatgcccatgatgacgaccctaacgaggaggctgtgaatttaatatttccagaatctatgattcttcaggctgacatagccaacgaatctactccacttcatacaccgactctgtcacccatacctgaattggaagaggaggacgaactagacctccggtgttatgaggaaggttttcctcccagcgattcagaggatgaacggggtgagcagaccatggctctgatctcagactatgcttgtgtgattgtggaggaacaagtagtgattgaaaattctaccgagccagtggagggctgtagaaaatgccagtaccaccgggataagtctggagacccgaacgcatcatgcgctttgtgctatatgaaacagactttcagctttatttacagtaagtggagtgaatgtgagagaggctgagtgcttaacacatcactgtgtattgcttgaacagctgtgctaagtgtggtttatttttgtttctaggtccggtgtcagaggatgagtcatcaccctcagaagaagaccacccgtctccccctgatctcacagatgacacgcccctgcaagtgcacagacccaccccagtcagagccagtggcgagaggcgagcagctgttgaaaaaattgaggacttgttacatgacatgggtggggatgaacctttggacctgagcttgaaacgccccaggaactaggcgcagctgcgcttagtcatgtgtaaataaagttgtacaataaaagtatatgtgacgcatgcaaggtgtggtttatgactcatgggcggggcttagtcctatataagtggcaacacctgggcactgggcacagaccttcagggagttcctgatggatgtgtggactatccttgcagactttagcaagacacgccggcttgtagaggatagttcagacgggtgctccgggttctggagacactggtttggaactcctctatctcgcctggtgtacacagttaagaaggattataaagaggaatttgaaaatatttttgctgactgctctggcctgctagattctctgaatcttggccaccagtcccttttccaggaaagggtactccacagccttgatttttccagcccagggcgcactacagccggggttgcttttgtggtttttctggttgacaaatggagccaggacacccaactgagcaggggctacatcctggacttcgcagccatgcacctgtggagggcctggatcaggcagcggggacagagaatcttgaattactggcttctacagccagcagctccgggtcttcttcgtctacacagacaaacatccatgttggaggaagaaatgaggcaggccatggacgagaacccgaggagcggcctggaccctccgtcggaagaggagctggattgaatcaggtatccagcctgtacccagagcttagcaaggtgctgacatccatggccaggggagttaagagggagaggagcgatgggggtaataccgggatgatgaccgagctgacggccagcctgatgaatcggaagcgcccagagcgccttacctggtacgagctacagcaggagtgcagggatgagttgggcctgatgcaggataaatatggcctggagcagataaaaacccattggttgaacccagatgaggattgggaggaggctattaagaagtatgccaagatagccctgcgcccagattgcaagtacatagtgaccaagaccgtgaatatcagacatgcctgctacatctcggggaacggggcagaggtggtcatcgataccctggacaaggccgccttcaggtgttgcatgatgggaatgagagcaggagtgatgaatatgaattccatgatcttcatgaacatgaagttcaatggagagaagtttaatggggtgctgttcatggccaacagccacatgaccctgcatggctgcagtttcttcggcttcaacaatatgtgcgcagaggtctggggcgcttccaagatcaggggatgtaagttttatggctgctggatgggcgtggtcggaagacccaagagcgagatgtctgtgaagcagtgtgtgtttgagaaatgctacctgggagtctctaccgagggcaatgctagagtgagacactgctcttccctggagacgggctgcttctgcctggtgaagggcacagcctctctgaagcataatatggtgaagggctgcacggatgagcgcatgtacaacatgctgacctgcgattcgggggtctgccatatcctgaagaacatccatgtgacctcccaccccagaaagaagtggccagtgtttgagaataacctgctgatcaagtgccatatgcacctgggagccagaaggggcaccttccagccgtaccagtgcaactttagccagaccaagctgctgttggagaacgatgccttctccagggtgaacctgaacggcatctttgacatggatgtctcggtgtacaagatcctgagatacgatgagaccaagtccagggtgcgcgcttgcgagtgcgggggcagacacaccaggatgcagccagtggccctggatgtgaccgaggagctgagaccagaccacctggtgatggcctgtaccgggaccgagttcagctccagtggggaggatacagattagaggtaggtttgagtagtgggcgtggctaaggtgactataaaggcgggtgtcttacgagggtctttttgcttttctgcagacatcatgaacgggactggcggggccttcgaaggggggctttttagcccttatttgacaacccgcctgccgggatgggccggagttcgtcagaatgtgatgggatcgacggtggatgggcgcccagtgcttccagcaaattcctcgaccatgacctacgcgaccgtggggaactcgtcgctcgacagcaccgccgcagccgcggcagccgcagccgccatgacagcgacgagactggcctcgagctacatgcccagcagcggtagtagcccctctgtgcccagttccatcatcgccgaggagaaactgctggccctgctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgtgggtggaggtagcaccactgcatggcctcgtgctctggggtcgtgttgtagatgatccagtcatagcaggggcgctgggcgtggtgctggatgatgtccttgaggaggagactgatggccacggggagccccttggtgtaggtgttggcaaaacggttgagctgggagggatgcatgcggggggagatgatgtgcagtttggcctggatcttgaggttggcgatgttgccacccagatcccgccgggggttcatgttgtgcaggaccaccagaacggtgtagcccgtgcacttggggaacttgtcatgcaacttggaagggaatgcgtggaagaatttggagacgcccttgtgcccgcccaggttttccatgcactcatccatgatgatggcaatgggcccgtgggctgcggctttggcaaagacgtttctggggtcagagacatcgtaattatgctcctgggtgagatcatcataagacattttaatgaatttggggcggagggtgccagattgggggacgatggttccctcgggccccggggcgaagttcccctcgcagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcttgtctctgacttggaggttttcccggacgagctcgccgaggaggcggtccccgcccagcgagagaagctcttgcagggaagcaaagtttttcaggggcttgagcccgtcggccatgggcatcttggcgagggtctgcgagaggagctccaggcggtcccagagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgcgtgagggtggtctccgtcacggtgaaggggtgggccgcgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaggcgtccgctccgcagtgggcgcagacggtctcgcactcgactagccaggtgagctcgggctgctcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcagaccactctgagacgaaggcgcgcgtccacgccaagacaaaggaggccacgtgcgaggggtagcggtcgttgtccaccagggggtccaccttttccacggtatgcaggcacatgtccccctcctccgcatccaagaaggtgattggcttgtaggtgtaggccacgtgacctggggttcccgacgggggggtataaaagggggcgggtctgtgctcgtcctcactctcttccgcgtcgctgtccacgagcgccagctgttggggtaggtattccctctcaagagcgggcatgacctcggcactcaggttgtcagtttctagaaacgaggaggatttgatgtgggcctgccctgccgcgatgctttttaggagactttcatccatctggtcagaaaagactatttttttattgtcaagcttggtggcgaaggagccatagagggcgtttgagagaagcttggcgatggatctcatggtctgatttttgtcacggtcggcgcgctccttggccgcgatgttgagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtctgccgcccttgcgcgagcagaacgggggcagcacatcaagcagatgctcgtcaggggggtccgcatcgatggtgaagatgcccggacagagttccttgtcaaaataatcgatttttgaggatgcatcgtccaaggccatctgccactcgcgggcggccagcgctcgctcgtaggggttgaggggcggaccccaaggcatgggatgcgtgagggcggaggcgtacatgccgcagatgtcatagacatagatgggctccgagaggatgccgatgtaggtgggatagcagcgccccccgcggatgcttgcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtgaggcaggcggaccgagtcgcggatgaagtgcgcgtaggagtcttgcagcttggcgacgagctcggcggtgacgaggacgtccatggcgcagtagtccagcgtttcgcggatgatgtcataactcgcctctcctttcttctcccacagctcgcggttgagggcgtattcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcaaaggtgtccctgaccatgactttcaagaactggtacttgaagtccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcaaagccgttgatgttgtgcccgacgatgtagagttccatgaatcgcgggcggcctttgatgtgcggcagctttttgagctcctcgtaggtgaggtcctcggggcattgcaggccgtgctgctcgagcgcccactcctggagatgtgggttggcttgcatgaaggaagcccagagctcgcgggccatgagggtctggagctcgtcgcgaaagaggcggaactgctggcccacggccatcttttctggggtgacgcagtagaaggtgagggggtcccgctcccagcgatcccagcgtaaacgcacggcgagatcgcgagcgagggcgaccagctctgggtccccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggtcgtgtccacgcattgcatgagcccgtagctgcgcggggccacgaccgtgccgcggtgcgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctctgcgtcattgacggcggcctgacgcaggatctcttgcacgtcgcccgagttgtcctggtaggcgatctcggacatgaactgttcgatctcctcctcctggagatcgccgcggcccgcgcgctccacggtggcggcgaggtcattggagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcaaagacggcgtagttgcgcaggcgctggaagaggtagttgagggtggtggcgatgtgctcggtgacgaagaagtacatgatccagcggcgcaggggcatctcgctgatgtcgccgatggcttccagcctttccatggcctcgtagaagtccacggcgaagttgaaaaactgggcgttgcgggccgagaccgtgagctcgtcttccaggagccggatgagttcggcgatggtggcgcgcacctcgcgctcgaaatccccgggggcctcctcctcttcctcttcttccatgacgacctcttcttctatttcttcctctgggggcggtggtggtggcgggggccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccattgggcagcgatagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgcggacgctgttagaattgcggttgctgatgatgtaattgaagtaggcgtttttgaggcggcggatggtggcgaggaggaccaggtccttgggtccagcttgctggatgcggagccgctcggccatgccccaggcctggccctgacaccggctcaggttcttgtagtagtcatgcatgagcctctcaatgtcatcactggctgaggcggagtcttccatgcgggtgaccccgacgcccctgagcggctgcacgagcgccaggtcggcgacgacgcgctcggcgaggatggcctgttgcacgcgggtgagggtgtcctggaagtcgtccatgtcgacgaagcggtgataggccccggtgttgatggtgtaggtgcagttggccatgagcgaccagttgacggtctgcaggcctggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccgagcccgatagccgccaggatacggcggagagccctttttgctggccgaggggggtcgctagacttgaaagcgaccgaaaaccctgccgggtagtggctcgcgcccgtagtctggagaagcatcgccagggttgagtcgcggcagaacccggttcgaggacggccgcggcgagcgggacttggtcaccccgccgatataaagacccacagccagccgacttctccagttacgggagcgagcccccttttttctttttgccagatgcatcccgtcctgcgccaaatgcgtcccacccccccggcgaccaccgcgaccgcggccgtagcaggcgccggcgctagccagccaccacagacagagatggacttggaagagggcgaagggctggcaagactgggggcgccgtccccggagcgacatccccgcgtgcagctgcagaaggacgtgcgcccggcgtacgtgcctacgcagaacctgttcagggaccgcagcggggaggagcccgaggagatgcgcgactgccggtttcgggcgggcagggagctgcgcgagggcctggaccgccagcgcgtgctgcgcgacgaggatttcgagccgaacgagcagacggggatcagccccgcacgcgcgcacgtggcggcagccaacctggtgacggcctacgagcagacggtgaagcaggagcgcaacttccaaaagagtttcaacaaccacgtgcgcaccctgatcgcgcgcgaggaggtggccctgggcctgatgcacctgtgggacctggcggaggccatcgtgcagaacccggacagcaagcctctgacggcgcagctgttcctggtggtgcagcacagcagggacaacgaggcgttcagggaggcgctgctgaacatcgccgagcccgagggtcgctggctgctggagctgattaacatcttgcagagcatcgtagtgcaggagcgcagcctgagcctggccgagaaggtggcggcgatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgacctgggcgtgtaccgcaacgaccgcatccacaaggccgtgagcacgagccggcggcgcgagctaagcgaccgcgagctgatgctgagtctgcgccgggcgctggtagggggcgccgccggcggcgaggagtcctacttcgacatgggtgcggacctgcattggcagccgagccggcgcgccttggaggccgcctacggttcagaggacttggatgaggaagaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacgaacgtgcagtccaacctggatcggctggtgacggacgtgcgcgaggccgtggcgcagcgcgagcggttcaagaacgagggcctgggctcgctggtggcgctgaacgccttcctggcaacgcagccggcgaacgtgccgcgcgggcaggacgattacaccaactttatcagcgcgctgcggctgatggtgaccgaggtgccccagagcgaggtgtaccagtctggcccggactactttttccagacgagccggcagggcttgcagacggtgaacctgagccaggctttcaagaatctgcgcgggctgtggggcgtgcaggcgcccgtgggcgaccggtcaacggtgagcagcttgctgacgcccaactcgcggctgctgctgctgctgatcgcgcccttcaccgacagcggcagcgtgaaccgcaactcgtacctgggccatctgctgacgctgtaccgcgaggccataggccaggcgcaggtggacgagcagaccttccaggagatcactagcgtgagccgcgcgctggggcagaacgacaccgacagtctgagggccaccctgaactttttgctgaccaatagacagcagaagatcccggcgcagtacgcactgtcggccgaggaggaaaggattctgagatatgtgcagcagagcgtagggctgttcctgatgcaggagggtgccacccccagcgccgcgctggacatgaccgcgcgcaacatggaacctagcatgtacgccgccaaccggccgttcatcaataagctgatggactacttgcaccgcgcggcggccatgaacacggactactttaccaacgccatcctgaacccgcactggctcccgccgccggggttctacacgggcgagtacgacatgcccgaccccaacgacgggttcctgtgggacgacgtggacagcgcggtgttctcgccgacctttcaaaagcgccaggaggcgccgccgagcgagggcgcggtggggaggagcccctttcctagcttagggagtttgcatagcttgccgggctcggtgaacagcggcagggtgagccggccgcgcttgctgggcgaggacgagtacctgaacgactcgctgctgcagccgccgcgggccaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggtcaacccgttcgcgcatctgcagcccaaactggggcgacggatgttttgaatgaaataaaactcaccaaggccatagcgtgcgttctcttccttgttagagatgaggcgcgcggtggtgtcttcctctcctcctccctcgtacgagagcgtgatggcgcaggcgaccctggaggttccgtttgtgcctccgcggtatatggctcctacggagggcagaaacagcattcgttactcggagctggctccgcagtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacactaacatgcccaatgtgaacgagtacatgttcaccagcaagtttaaggcgcgggtgatggtgtctaggaagcatccagagggggtagttgaaacagatttgagtcaggataagcttgaatatgagtggtttgagtttaccctgcccgagggaaacttttccgagaccatgaccatagacctgatgaacaacgccatcttggaaaactacttgcaagtggggcggcagaatggcgtgctggagagcgatatcggagtcaagtttgacagcagaaatttcaagctgggctgggacccggtgaccaagctggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaggatctagaaggtggcaacatccccgccctccttgatgtgcccaagtacttggaaagcaagaagaaagttgaagacgaaactaaaaatgcagctgcggccacagccgatacaaccactaggggtgatacatttgcaactccagcgcaagagacagcagctgataagaaggtagaagtcttgcccattgaaaaggatgagagtggtagaagttacaacctgatccaggggacccacgacacgctgtaccgcagttggtacctgtcctatacctacggggaccccgagaagggggtgcagtcgtggacgctgctcaccaccccggacgttacctgcggcgcggagcaagtctactggtcactgccggacctcatgcaagaccccgtcaccttccgctccacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctttccagtcgcaccttctaaaaaaatgtctattctcatctcgcccagcaataacaccggctggggtcttactagacccagcaccatgtacggaggagccaagaagcgctcccagcagcaccccgtccgcgtccgcggccacttccgcgctccctggggcgcttacaagcgcgggcggacttccaccgccgtgcgcaccaccgtcgacgacgtcatcgactcggtggtcgccgacgcgcgcaactacactcccgccccctccaccgtggacgcggtcatcgacagcgtggtggccgacgcgcgcgactatgccagacgcaagagccggcggcgacggatcgccaggcgccaccggagcacgcccgccatgcgcgccgcccgggctctgctgcgccgcgccagacgcacgggccgccgggccatgatgcgagccgcgcgccgcgctgccactgcacccacccccgcaggcaggactcgcagacgagcggccgccgccgccgctgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaccccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggtcaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcaaaccgtggacattcagatccccaccgacatggatgtcgacaaaaaaccctcgaccagcatcgaggtgcaaaccgacccctggctcccagcctccaccgctaccgtctccacttctaccgccgccacggctaccgagcctcccaggaggcgaagatggggcgccgccagccggctgatgcccaactacgtgttgcatccttccatcatcccgacgccgggctaccgcggcacccggtactacgccagccgccggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaattcgtgtgctgtgatactgttgcagagagatggctctcacttgccgcctgcgcatccccgtcccgaattaccgaggaagatcccgccgcaggagaggcatggcaggcagcggcctgaaccgccgccggcggcgggccatgcgcaggcgcctgagtggcggctttctgcccgcgctcatccccataatcgccgcggccattggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccaaaacttccagcagaaggtggtggacgggctggcctcgggcattaacggggtggtggacatcgcgaaccaggccgtgcagcgcgagataaacagccgcctggacccgcggccgcccacggtggtggagatggaagatgcaactcttccgccgcccaaaggcgaaaagcggccgcggcccgacgcggaggagacgatcctgcaggtggacgagccgccctcgtacgaggaggccgtcaaggccggcatgcccaccacgcgcatcatcgcgccgctggccacgggtgtaatgaaacccgccacccttgacctgcctccaccacccgcgcccgctccaccgaaggcaactccggttgtgcaggcccccccggtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagcctggctcccaagggtgcccccaatcccagtcagtgggaaacaaaagaaaagcaaggaactactggaggagtgcagcaagaaaaagatgtcacaaaaacatttggtgtggctgccaccggcggaattaatataacaaaccagggtctgttactaggaactgacgaaaccgctgagaatggcaaaaaagacatttatgcagacaagactttccagccagaacctcaagttggagaagaaaactggcaggaaaatgaagccttctatggaggaagggctcttaaaaaggacactaaaatgaaaccatgctatggatcttttgctagacctactaatgagaaaggaggtcaggcaaagttcaaaccagttaatgaaggagaacaacctaaagatctggatatagattttgcttactttgacgtccctggcggaagtcctccagcaggtggtagtggggaagaatacaaagcagatataattttgtacactgaaaatgttaatcttgaaacaccagacactcatgtggtttacaagccaggaacttcagataacagttcagaaatcaatctggttcagcagtccatgccaaacagacccaactacattggctttagggacaactttgtaggtctcatgtattacaacagcaccggaaatatgggtgtgctggctggtcaggcttctcagttgaacgctgtggtcgacttgcaagacagaaacaccgagttatcttaccagctattgctagattctctgggtgacagaaccagatactttagcatgtggaactctgcggtggacagttacgatccagatgtcaggatcattgaaaatcacggtgtggaagatgaacttccaaactattgcttcccattgaatggcactggaaccaattccacttatcaaggtgtaaagattacaaatggtaatgatggtgctgaagaaagtgagtgggagaaagacgatgcaatttctagacaaaaccaaatctgcaagggcaatgtctacgccatggagatcaacctgcaggccaacctgtggaagagttttctgtactcgaacgtggccctgtacctgcccgactcctacaagtacacgccggccaacgtcaagctgcccgccaacaccaacacctacgagtacatgaacggccgcgtggtagccccatccctggtggacgcctacatcaacatcggcgcccgctggtcgttggaccccatggacaacgtcaaccccttcaaccaccaccgcaatgcgggcctgcgctaccgctccatgctgctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgcttcgacagcgtcaacctatacgccactttcttccccatggcgcacaacaccgcttcaaccttggaagccatgctgcgcaacgacaccaacgaccagtccttcaacgactacctctcggccgccaacatgctctaccccatcccggccaaggccaccaacgtgcccatctccatcccatcgcgcaactgggccgccttccgcggctggagtttcacccggctcaagaccaaggaaactccttccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaataactcgggcttcaccggctacctcgcacccaccatgcgccaggggcagccctaccccgccaacttcccctacccgctcatcggtcagacagccgtgccctccgtcacccagaaaaagttcctctgcgacagggtcatgtggcgcatcccattctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtacaccagccgcaccgcggcgtcatcgaggccgtctacctgcgcacgcccttctccgccggcaacgccaccacctaagcatgagcggctccagcgaacgagagctcgcggccatcgtgcgcgacctgggctgcgggccctactttttgggcacccacgacaagcgcttcccgggctttctcgccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgcagcgccctggcctcctcgcccgaccgctgtctcagcctcgagcagtccactcagaccgtgcaggggcccgactccgccgcctgcggactcttctgttgcatgttcttgcatgccttcgtgcactggcccgaccgacccatggacggaaaccccaccatgaacttgctgacgggggtgcccaacggcatgctacaatcgccacaggtgctgcccaccctcaggcgcaaccaggaggaactctaccgcttcctcgcgcgccactccccttactttcgctcccaccgcgccgccatcgaacacgccaccgcttttgacaaaatgaaacaactgcgtgtatctcaataaacagcacttttattttacatgcactggagtatatgcaagttatttaaaagtcgaaggggttctcgcgctcgtcgttgtgcgccgcgctggggagggccacgttgcggtactggtacttgggctgccacttgaactcggggatcaccagtttgggcactggggtctcggggaaggtctcgctccacatgcgccggctcatctgcagggcgcccagcatgtccggggcggagatcttgaaatcgcagttggggccggtgctctgcgcgcgcgagttgcggtacacggggttgcagcactggaacaccatcagactggggtacttcacactagccagcacgctcttgtcgctgatctgatccttgtccagatcctcggcgttgctcaggccgaacggggtcatcttgcacagctggcgtcccaggaagggcacgctctgaggcttgtggttacactcgcagtgcacgggcatcagcatcatccccgcgccgcgctgcatattcgggtagagggccttgacaaaggccgcgatctgcttgaaagcttgctgggccttggccccctcgctgaaaaacaggccgcagctcttcccgctgaactggttattcccacacccggcatcctgcacgcagcagcgcgcgtcatggctggtcagttgcaccacgctccgtccccagcggttctgggtcaccttagccttgctgggctgctccttcaacgcgcgctgcccgttctcgctggtcacatccatctccaccacgtggtccttgtggatcatcatcgtcccgtgcagacacttgagctggccttccacctcggtgcagccgtgatcccacagggcgcaaccggtgcactcccagttcttgtgcgcaatcccgctgtggctgaagatgtaaccttgcaacatgcggcccatgatggtgctaaatgctttctgggtggtgaaggtcagttgcatcccgcgggcctcctcgttcatccaggtctggcacatcttctggaagatctcggtctgctcgggcatgagcttgtaagcatcgcgcaggccgctgtcgacgcggtagcgttccatcagcacgttcatggtatccatgcccttctcccaggacgagaccagaggcagactcagagggttgcgtacgttcaggacaccgggggtcgcgggctcgacgatgcgttttccgtccttgccttccttcaatagaaccggcggctggctgaatcccactcccacgatcacggcatcttcctggggcatctcttcgtcggggtctaccttggtcacatgcttggtctttctggcttgcttcttttttggagggctgtccacggggagcacgtcctcctcggaagacccggagcccacccgctgatactttcggcgcttggtgggcagaggaggtggcggcgaggggctcctctcctgctccggcggatagcgcgccgacccgtggccccggggcggagtggcctctcggcccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctcgagcatggctacctgggaggagaggaggatgtgctgctgaaacacctgcagcgccagtccctcatcctccgggacgccctggccgaccggagcgaaacccccctcagcgtcgaggagctgtgtcgggcctacgagctcaacctcttctcgccgcgcgtaccccccaaacgccagcccaacggcacctgcgagcccaacccgcgtctcaacttctatcccgtctttgcggtccccgaagccctcgccacctatcacatctttttcaagaaccaaaagatccccgtctcctgccgcgccaaccgcaccagcgccgacgcgctcctcgctctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagctcatcatgccccacatcgaggccctcgatgaaagtcaggagcagcgccccgaggacgcccggcccgtggtcagcgacgagatgctcgcgcgctggctcgggacccacgacccccaggctttggaacagcggcgcaagctcatgctggccgtggtcctggttaccctcgagctggaatgcatgcgccgcttcttcagcgaccccgagaccctgcgcaaggtcgaggagaccctgcactacactttcagacacggtttcgtcaggcaggcctgcaagatctccaacgtggagctgaccaacctggtctcctgcctggggatcctgcacgagaaccgcctggggcagaccgtgctccactctaccctgaagggcgaggcgcggcgggactatgtccgcgactgcgtctttctatttctttgccacacatggcaagcagccatgggcgtgtggcaacagtgtctcgaggacgataacctgaaggagctggacaagcttcttgctagaaatcttaaaaagctgtggacgggcttcgacgagcgcaccgtcgcctcggacctggccgagatcgtgttccccgagcgcctgaggcagacgctgaaaggcgggctgcccgacttcatgagccagagcatgttgcaaaactaccgcactttcattctcgagcgatctgggatgctgcccgccacctgcaacgctttcccctccgactttgtcccgctgagctaccgcgagtgtcccccgccgctgtggagccactgctacctcttgcagctggccaactacatcgcctaccactcggacgtgatcgaggacgtgagcggcgaggggctgctcgagtgccactgccgctgcaacctgtgctccccgcaccgctccctggtctgcaacccccagctactaagcgagacccaggtcatcggtacctttgagctgcaaggtccgcaggagtccaccgctccgctgaaactcacgccggggttgtggacttccgcgtacctgcgcaaatttgtacccgaggactaccacgcccacgagataaagttcttcgaggaccaatcgcgtccgcagcacgcggatctcacggcctgcgtcatcacccagggcgcaatcctcgcccaattgcacgccatccaaaaatcccgccaagagtttcttctgaaaaagggtagaggggtctacctggacccccagacgggcgaagtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagaggagcaggcaacagagcagcccgtcgccgcaccatccgcgccggcagccccgccggtcacggatacaacctccgcagctccggccaagcctcctcgtagatgggatcgagtgaagggtgacggtaagcacgagcggcagggctaccgatcatggagggcccacaaagccgcgatcatcgcctgcttgcaagactgcggggggaacatcgctttcgcccgccgctacctgctcttccaccgcggggtaaacatcccccgcaacgtgttgcattactaccgtcaccttcacagctaagaaaaagcaagtaaaaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccaaatgggcctggccgcgggcgcctcccaggactactccacccgcatgaactggctcagtgccggcccctcgatgatctcacgggtcaacggggtccgcagtcatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagctcaacgacgagttggtgagctcttcgatcggtctgcgaccggacggagtgttccaactagccggagccgggagatcctccttcactcccaaccaggcctacctgaccttgcagagcagctcttcggagcctcgctccggaggcatcggaaccctccagtttgtggaggagtttgtgccctcggtctacttcaaccccttctcgggatcgccaggcctctacccggacgagttcataccgaacttcgacgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtccaaccccgaagttgcatgagaatttttgttgtactctttgtggtgagtttaataaaagctaaactcttgcaatactctggaccttgtcgtcgtcaactcaacgagaccgtctacctcaccaaccagactgaggtaaaactcacctgcagaccacacaagacctatatcatctggttcttcgagaacacctcatttgcagtctccaacactcactgcaacgacggtgttgaacttcccaacaacctttccagtggactgagttacgatacacatagagctaagctcgtcctctacaatccttttgtagagggaacctaccagtgccagagcggaccttgtactcacaccttccatttggtgaacgtcaccagcagcagcaacagctcagaaactaaccttccttctgatactaacaaacctcgtttcggaggtgagctaaggcttcccccttctgaggagggggttagcccatacgaagtggtcgggtatttgattttaggggtggtcctgggtgggtgcatagcggtgctagctcagctgccttgctgggtggaaatcaaaatctttatatgctgggtcagatattgtggggaggaaccatgaaggggcttttgctgattatccttttcatggtggggggtgtactgtcatgccacgaacagccacgatgtaacatcaccacaggcaatcatatgagcagagagtgcactgtagtcatcaaatgcgagcacgactgcccactaaacattacattcaagaataacaccatgggaaatgtatgggtgggtttctgggaaccaggagatgagcagaactacacggtcactgtccatggtagcaatggaaatcacactttcggtttcaaattcatttttgaagtcatgtgtgatatcacactgcatgtggctagacttcatggcttgtggccccctaccaaggagaacatggttgggttttctttggcttttgtgatcatggcctgcttgatgtcaggtctgctggtaggggctttagtgtggttcctgaagcgcaagcctaggtacggaaatgaagaaaaggaaaaattgctataatctttttctttttcacagaaccatgaatgctttgaccagtgtcgtgctgctctctcttcttgtagcttttagtaatggggaagctgaaactgtagttgtaaatgttaaatctggtacaaaccacacccttgaaggtcctagaaaaactccagttcagtggtatgggggtgctaactttgacatgttttgcaatggctctaaaatacatcacaatgaattgaatcacacttgctctattcagaacataactcttacattcataaacagaacacatcatggaacatactatggttttggctctgacaatcaaaattcaaaagtgtatcatgtcagagtagatgtagagcctcctagaccccgtgctactttggctcctcctcaggacataactattaagtatggctcaaatagaacattgcagggcccaagtgttactccagttagttggtatgatggtgaaggaaatcggttttgcgatggcgataaaattgatcatacagaaattaatcacacttgcaatgctcaaaaccttactttgctgtttgtgaatgaaacacatgaaagaacatattatggaattagtggtgattggaaacagcgaaatgagtatgatgttactgttacaaagacacaattaaatattaaaaatttgggccaacgcaaaactgatgaaaaccataaaaatggaatgcatcagaaagtcgaacaaaatcctgaaactaagaaagaacagaagccttcaaaaagacctagacaaaaaacattgcaaactacaattcaggttatgattcctattggaactaattatactttagtggggccttcgccaccagtgagctggcatactacaaaaaatggcttaacagaactctgtaatggaaaccctattttaagacacacttgtgatgggcaaaatattacacttattaatgttaatgctacatttgaggctgattactatggctcgaacaataagagtgaatcaaaacactacagagtcaaggttttcaaagaaagaaaagatcaggcactattattcagaccgcttactaccaaaggaagcatgatcattactactgaaaatcaaaactttgaattacaacaaggtgacaatcaagatgatgacaaaattccatcaactactgtggcaatcgtggtgggtgtgattgcgggctttgtgactctgatcattgtcttcatatgctacatctgctgccgcaagcgtcccaggtcatacaatcatatggtagacccactactcagcttctcttactgaaactcagtcactctcatttcagaaccatgaaggctttcacagcttgcgttctgattagcatagtcacacttagtgcagctgaagctaaatgctttcatacttataacttaactagaggggaaaatattacattagcaggtgctggcttaaacacaacatgggaagcatatcacaatggatggaaacaagtttgtccatggaatgacggtcgctatgtgtgcgttggaaacagcagtaccataactaatcttacagttgtagctaatgcaaatttatcatcaactgttaaatttagagctgaaagtttatacattggaacagatggatatgaaagcaatccatcatgcttttatactatcaatgtaattgagcttccaaccaccagatcgccaactaccaccacggtcagtacaactactgagaccacaactcacactacacagttagacactacagtgcagaatagtactgtattggttaggtatttgttaagggaggaaagtactactgaacagacagaggctacctcaagcgccttcagcagcacttcaaatttaacttcgcttgcttggactaatgaaaccggagtatcattgatgcatggccagccttactcaggtttggatattcaaattacttttctggttgtctgtgggatctttattcttgtggttcttctgtactttgtctgctgcaaagccagagaaaaatctaggcggcccatctacaggccagtaatcggggaacctcagccactccaagtggatggaggcttaaggaatcttcttttctcttttacagtatggtgatcagccatgattcctaggttcttcctatttaacatcctcttctgtctcttcaacgtgtgcgctgccttcgcggccgtctcgcacgcctcacccgactgtctcgggcccttccccacctacctcctctttgccctgctcacctgcacctgcgtctgcagcattgtctgcctggtcatcaccttcctgcagctcatcgactggtgctgcgcgcgctacaattacctgcatcatagtcccgaatacagggacgagaacgtagccagaatcttaaggctcatatgaccatgcagactctgctcatactgctatccctcctatcccctaccctcgccacttctgctgattactctaaatgcaaattcgcggacatatggaatttcttagactgctatcaggagaaaattgacatgccctcctattacttggtgattgtgggaatagttatggtctgctcctgcactttctttgccatcatgatctacccctgttttgatctcggctggaactctgttgaagcattcacatacacactagaaagcagttcactagcctccacgccaccacccacaccgcctccccgcagaaatcagtttcccatgattcagtacttagaagagccccctccccgacccccttccactgttagctactttcacataaccggcggcgatgactgaccaccacctggacctcgagatggacggccaggcctccgagcagcgcatcctgcaactgcgcgtccgtcagcagcaggagcgtgccgccaaggagctcctcgatgccatcaacatccaccagtgcaagaagggcatcttctgcctggtcaaacaggcaaagatcacctacgagctcgtgtccggcggcaagcagcatcgcctcgcctatgagctgccccagcagaagcagaagttcacctgcatggtgggcgtcaaccccatagtcatcacccagcagtcgggcgagaccagcggctgcatccactgctcctgcgaaagccccgagtgcatctactccctgctcaagaccctttgcggactccgcgacctcctccccatgaactgatgttgattaaaagcccaaaaaccaatcagccccttcccccatttccccatcccccaattactcataaaaaataaatcattggaattaatcattcaataaagatcacttacttgaaatctgaaagtatgtctctggtgtagttgttcagcagcacctcggtaccctcctcccagctctggtactccagtccccggcgggcggcgaacttcctccacaccttgaaagggatgtcaaattcctggtccacaattttcattgtcttccctctcagatggcaaagaggctccgggtggaagatgacttcaaccccgtctacccctatggctacgcgcggaatcagaatatccccttcctcactcccccctttgtctcctccgatggattcaaaaacttcccccctggggtcctgtcacttaaactggctgatccaatcaccatcaacaatggggatgtctcacttaaggtgggagggggacttgctgtagagcaacagactggtaacctaagcgtaaaccctgatgcacccttgcaagttgcaagtgataagctacagcttgctctggctcctccattcgaggtcagagatggaaagcttgctttaaaggcaggtaatggattaaaagtactagataattccattactggattgactggattattgaatacacttgtggtattaactggaaggggaataggaacggaggaattaaaaaatgacgatggtgtaacaaacaaaggagtcggcttgcgtgtaagacttggagatgacggcgggctgacatttgataaaaagggtgatttagtagcctggaataaaaaagatgacaggcgcaccctgtggacaacccctgacacatctccaaattgcaaaatgagtacagaaaaggattctaaacttacgttgacacttacaaagtgtggaagtcaggttctgggaaatgtatctttacttgcagttacaggtgaatatcatcaaatgactgctactacaaagaaggatgtaaaaatatctttactatttgatgagaatggaattctattaccatcttcgtcccttagcaaagattattggaattacagaagtgatgattctattgtatctcaaaaatataataatgcagttccattcatgccaaacctgacagcttatccaaaaccaagcgctcaaaatgcaaaaaactattcaagaactaaaatcataagtaatgtctacttaggtgctcttacctaccaacctgtaattatcactattgcatttaatcaggaaactgaaaatggatgtgcttattctataacatttaccttcacttggcaaaaagactattctgcccaacagtttgatgttacatcttttaccttctcatatcttacccaagagaacaaagacaaagactaataaaatgttttgaactgaatttatgaatctttatttatttttacaccagcacgggtagtcagtttcccaccaccagcccatttcacagtgtaaacaattctctcagcacgggtggccttaaatagggaaatgttctgattagtgcgggaactggacttggggtctataatccacacagtttcctggcgagccaaacgggggtcggtgattgagatgaagccgtcctctgaaaagtcatccaagcgggcctcacagtccaaggtcacagtctggtggaatgagaagaacgcacagattcatactcggaaaacaggatgggtctgtgcctctccatcagcgccctcaacagtctttgccgccggggctcggtgcggctgctgcagatgggatcgggatcgcaagtctctctgactatgatccccacagccttcagcaacagtctcctggtgcgtcgggcacagcaccgcatcctgatctctgccatgttctcacagtaagtgcagcacataatcaccatgttattcagcagcccataattcagggcgctccaaccaaagctcatgttggggatgatggaacccacgtgaccatcgtaccagatgcggcagtatatcaggtgcctgcccctcatgaacacactgcccatatacatgatctctttgggcatgtttctgttcacaatctgccggtaccatgggaatcgctggttgaacatgcacccgtaaatgactctcctgaaccacacggccagcatggtgcctcccgcccgacactgcagggatcccggggctgaacagtggcaatgcaggatccagcgctcgtacccgctcaccatctgagctctcaccaagtccagggtagcggggcacaggcacactgacatacatctttttaaaatttttatttcctctggggtcaggatcatatcccaggggactggaaactcttggagcagggtaaagccagcagcacatggtaatccacggacagaacttacattatgataatctgcatgatcacaatcgggcaacagggggtgttgttcagttagtgaggccctagtctcctcctcacatcgtggtaaacgggccctgcggtaaggatgatggcggagcgagctcgactgttcctcggtggacattgaaatggattctcttgcgtaccttgtcgtacttctgccagcagaaagtggctcgggaacagcagatacctttcctcctgctgtccttccgctgctgacgctcagtcatccaactgaagtacagccattcccgcaggttctccagcagctcctgtgcatctgatgaaacaaaagtcccgtcgatgcggattccccttaaaacatcagccaggacattgtaggccatcccaatccagttaatgcatcctgatctatcatgaagaggaggtgggggaagaactggaagaaccatttttattccaagcggtctcgaaggacgataaagtgcaagtcacgcaggtgacagcgttccccgccgctgtgctggtggaaacagacagccaggtcaaaacccactctattttcaaggtgctcgactgtggcttcgagcagtggctctacgcgtacatccagcataagaatcacattaaaggctggacctccatcgatttcatcaatcatcaggttacactcattcaccatccccaggtaattctcatttttccagccttggattatttctacaaattgttggtgtaagtccactccgcacatgtggaaaagttcccacagcgccccctccactttcataatcaggcagaccttcatattagaaacagatcctgctgctccaccacctgcagcgtgttcaaaacaacaagattcaatgaggttctgccctctgccctcagctcacgtctcagcgtcagctgcaaaaagtcactcaagtcctcagccactacagctgacaattcagagccagggctaagcgtgggactggcaagcgtgagtgagtactttaatgctccaaagctagcacccaaaaactgcatgctggaataagctctctttgtgtcaccggtgatgccttccaataggtgagtgataaagcgaggtagtttttctttaatcatttgagtaatagaaaagtcctctaaataagtcactaggaccccaggaaccacaatgtggtagctgacagcgtgtcgctcaagcatggttagtagagatgagagtctgaaaaacagaaagcatgcactaaaccagagttgccagtctcactgaaggaaaaatcactctctccagcagcaaagtgcccactgggtggccctctcggacatacaaaaatcgatccgtgtggttaaagagcagcacagttagctcctgtcttctcccagcaaagatcacatcggactgggttagtatgcccctggaatggtagtcattcaaggccataaatctgccttggtagccattaggaatcagcacgctcactctcaagtgaaccaaaaccaccccatgcggaggaatgtggaaagattctgggcaaaaaaaggtatatctattgctagtcccttcctggacgggagcaatccctccagggctatctatgaaagcatacagagattcagccatagctcagcccgcttaccagtagacagagagcacagcagtacaagcgccaacagcagcgactgactacccactgacccagctccctatttaaaggcaccttacactgacgtaatgaccaaaggtctaaaaaccccgccaaaaaaacacacacgccctgggtgtttttcgcgaaaacacttccgcgttctcacttcctcgtatcgatttcgtgactcaacttccgggttcccacgttacgtcacttctgcccttacatgtaactcagccgtagggcgccatcttgcccacgtccaaaatggcttccatgtccggccacgcctccgcggcgaccgttagccgtgcgtcgtgacgtcatttgcatcaccgtttctcgtccaatcagcgttggctccgccccaaaaccgttaaaattcaaaagctcatttgcatattaacttttgtttactttgtggggtatattattgatgatg

SEQ ID NO: 2 - DNA, 30729 bp, Artificial Sequence
  pAd26DE1DE4Ad5Orf6
ttaaacatcaataatataccccacaaagtaaacaaaagttaatatgcaaatgagcttttgaattttaacggttttggggcggagccaacgctgattggacgagaaacggtgatgcaaatgacgtcacgacgcacggctaacggtcgccgcggaggcgtggcctagcccggaagcaagtcgcggggctgatgacgtataaaaaagcggactttagacccggaaacggccgattttcccgcggccacgcccggatatgaggtaattctgggcggatgcaagtgaaattaggtcattttggcgcgaaaactgaatgaggaagtgaaaagcgaaaaataccggtccctcccagggcggaatatttaccgagggccgagagactttgaccgattacgtgggggtttcgattgcggtgtttttttcgcgaatttccgcgtccgtgtcaaagtccggtgtttatgtcacagcggccgcatttaaatggcgcgcctaggtttgagtagtgggcgtggctaaggtgactataaaggcgggtgtcttacgagggtctttttgcttttctgcagacatcatgaacgggactggcggggccttcgaaggggggctttttagcccttatttgacaacccgcctgccgggatgggccggagttcgtcagaatgtgatgggatcgacggtggatgggcgcccagtgcttccagcaaattcctcgaccatgacctacgcgaccgtggggaactcgtcgctcgacagcaccgccgcagccgcggcagccgcagccgccatgacagcgacgagactggcctcgagctacatgcccagcagcggtagtagcccctctgtgcccagttccatcatcgccgaggagaaactgctggccctgctggccgagctggaagccctgagccgccagctggccgccctgacccagcaggtgtccgagctccgcgaacagcagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgtgggtggaggtagcaccactgcatggcctcgtgctctggggtcgtgttgtagatgatccagtcatagcaggggcgctgggcgtggtgctggatgatgtccttgaggaggagactgatggccacggggagccccttggtgtaggtgttggcaaaacggttgagctgggagggatgcatgcggggggagatgatgtgcagtttggcctggatcttgaggttggcgatgttgccacccagatcccgccgggggttcatgttgtgcaggaccaccagaacggtgtagcccgtgcacttggggaacttgtcatgcaacttggaagggaatgcgtggaagaatttggagacgcccttgtgcccgcccaggttttccatgcactcatccatgatgatggcaatgggcccgtgggctgcggctttggcaaagacgtttctggggtcagagacatcgtaattatgctcctgggtgagatcatcataagacattttaatgaatttggggcggagggtgccagattgggggacgatggttccctcgggccccggggcgaagttcccctcgcagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcttgtctctgacttggaggttttcccggacgagctcgccgaggaggcggtccccgcccagcgagagaagctcttgcagggaagcaaagtttttcaggggcttgagcccgtcggccatgggcatcttggcgagggtctgcgagaggagctccaggcggtcccagagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgcgtgagggtggtctccgtcacggtgaaggggtgggccgcgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagaggagggattgcagggcgtagagcttgggcgcgagaaagacggactcgggggcgaaggcgtccgctccgcagtgggcgcagacggtctcgcactcgactagccaggtgagctcgggctgctcggggtcaaaaaccagttttcccccgttctttttgatgcgcttcttacctcgcgtctccatgagtctgtgtccgcgctcggtgacaaacaggctgtctgtgtccccgtagacggacttgatgggcctgtcctgcaggggcgtcccgcggtcctcctcgtagagaaactcagaccactctgagacgaaggcgcgcgtccacgccaagacaaaggaggccacgtgcgaggggtagcggtcgttgtccaccagggggtccaccttttccacggtatgcaggcacatgtccccctcctccgcatccaagaaggtgattggcttgtaggtgtaggccacgtgacctggggttcccgacgggggggtataaaagggggcgggtctgtgctcgtcctcactctcttccgcgtcgctgtccacgagcgccagctgttggggtaggtattccctctcaagagcgggcatgacctcggcactcaggttgtcagtttctagaaacgaggaggatttgatgtgggcctgccctgccgcgatgctttttaggagactttcatccatctggtcagaaaagactatttttttattgtcaagcttggtggcgaaggagccatagagggcgtttgagagaagcttggcgatggatctcatggtctgatttttgtcacggtcggcgcgctccttggccgcgatgttgagctggacatattcgcgcgcgacacacttccattcggggaagacggtggtgcgctcgtcgggcacgatcctgacgcgccagccgcggttatgcagggtgaccaggtccacgctggtggccacctcgccgcgcaggggctcgttggtccagcagagtctgccgcccttgcgcgagcagaacgggggcagcacatcaagcagatgctcgtcaggggggtccgcatcgatggtgaagatgcccggacagagttccttgtcaaaataatcgatttttgaggatgcatcgtccaaggccatctgccactcgcgggcggccagcgctcgctcgtaggggttgaggggcggaccccaaggcatgggatgcgtgagggcggaggcgtacatgccgcagatgtcatagacatagatgggctccgagaggatgccgatgtaggtgggatagcagcgccccccgcggatgcttgcgcgcacgtagtcatacaactcgtgcgagggggccaagaaggcggggccgagattggtgcgctggggctgctcggcgcggaagacgatctggcgaaagatggcgtgcgagttggaggagatggtgggccgttggaagatgttaaagtgggcgtgaggcaggcggaccgagtcgcggatgaagtgcgcgtaggagtcttgcagcttggcgacgagctcggcggtgacgaggacgtccatggcgcagtagtccagcgtttcgcggatgatgtcataactcgcctctcctttcttctcccacagctcgcggttgagggcgtattcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcaaaggtgtccctgaccatgactttcaagaactggtacttgaagtccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgaggacgatctcgtcaaagccgttgatgttgtgcccgacgatgtagagttccatgaatcgcgggcggcctttgatgtgcggcagctttttgagctcctcgtaggtgaggtcctcggggcattgcaggccgtgctgctcgagcgcccactcctggagatgtgggttggcttgcatgaaggaagcccagagctcgcgggccatgagggtctggagctcgtcgcgaaagaggcggaactgctggcccacggccatcttttctggggtgacgcagtagaaggtgagggggtcccgctcccagcgatcccagcgtaaacgcacggcgagatcgcgagcgagggcgaccagctctgggtccccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaggcgggtgagggcgtgcttgagatgcagatggtacttgatttctacgggtgagttggtggtcgtgtccacgcattgcatgagcccgtagctgcgcggggccacgaccgtgccgcggtgcgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctctgcgtcattgacggcggcctgacgcaggatctcttgcacgtcgcccgagttgtcctggtaggcgatctcggacatgaactgttcgatctcctcctcctggagatcgccgcggcccgcgcgctccacggtggcggcgaggtcattggagatgcgacccatgagctgcgagaaggcgcccaggccgctctcgttccagacgcggctgtagaccacgtccccgtcggcgtcgcgcgcgcgcatgaccacctgcgcgaggttgagctccacgtgccgcgcaaagacggcgtagttgcgcaggcgctggaagaggtagttgagggtggtggcgatgtgctcggtgacgaagaagtacatgatccagcggcgcaggggcatctcgctgatgtcgccgatggcttccagcctttccatggcctcgtagaagtccacggcgaagttgaaaaactgggcgttgcgggccgagaccgtgagctcgtcttccaggagccggatgagttcggcgatggtggcgcgcacctcgcgctcgaaatccccgggggcctcctcctcttcctcttcttccatgacgacctcttcttctatttcttcctctgggggcggtggtggtggcgggggccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccattgggcagcgatagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgcggacgctgttagaattgcggttgctgatgatgtaattgaagtaggcgtttttgaggcggcggatggtggcgaggaggaccaggtccttgggtccagcttgctggatgcggagccgctcggccatgccccaggcctggccctgacaccggctcaggttcttgtagtagtcatgcatgagcctctcaatgtcatcactggctgaggcggagtcttccatgcgggtgaccccgacgcccctgagcggctgcacgagcgccaggtcggcgacgacgcgctcggcgaggatggcctgttgcacgcgggtgagggtgtcctggaagtcgtccatgtcgacgaagcggtgataggccccggtgttgatggtgtaggtgcagttggccatgagcgaccagttgacggtctgcaggcctggctgcacgacctcggagtacctgagccgcgagaaggcgcgcgagtcgaagacgtagtcgttgcaggtgcgcacgaggtactggtatccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaatagtccatggtcggcacggtctggccggtgagacgcgcgcagtcattgacgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccgagcccgatagccgccaggatacggcggagagccctttttgctggccgaggggggtcgctagacttgaaagcgaccgaaaaccctgccgggtagtggctcgcgcccgtagtctggagaagcatcgccagggttgagtcgcggcagaacccggttcgaggacggccgcggcgagcgggacttggtcaccccgccgatataaagacccacagccagccgacttctccagttacgggagcgagcccccttttttctttttgccagatgcatcccgtcctgcgccaaatgcgtcccacccccccggcgaccaccgcgaccgcggccgtagcaggcgccggcgctagccagccaccacagacagagatggacttggaagagggcgaagggctggcaagactgggggcgccgtccccggagcgacatccccgcgtgcagctgcagaaggacgtgcgcccggcgtacgtgcctacgcagaacctgttcagggaccgcagcggggaggagcccgaggagatgcgcgactgccggtttcgggcgggcagggagctgcgcgagggcctggaccgccagcgcgtgctgcgcgacgaggatttcgagccgaacgagcagacggggatcagccccgcacgcgcgcacgtggcggcagccaacctggtgacggcctacgagcagacggtgaagcaggagcgcaacttccaaaagagtttcaacaaccacgtgcgcaccctgatcgcgcgcgaggaggtggccctgggcctgatgcacctgtgggacctggcggaggccatcgtgcagaacccggacagcaagcctctgacggcgcagctgttcctggtggtgcagcacagcagggacaacgaggcgttcagggaggcgctgctgaacatcgccgagcccgagggtcgctggctgctggagctgattaacatcttgcagagcatcgtagtgcaggagcgcagcctgagcctggccgagaaggtggcggcgatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgacctgggcgtgtaccgcaacgaccgcatccacaaggccgtgagcacgagccggcggcgcgagctaagcgaccgcgagctgatgctgagtctgcgccgggcgctggtagggggcgccgccggcggcgaggagtcctacttcgacatgggtgcggacctgcattggcagccgagccggcgcgccttggaggccgcctacggttcagaggacttggatgaggaagaggaagaggaggaggatgcacccgctgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacgaacgtgcagtccaacctggatcggctggtgacggacgtgcgcgaggccgtggcgcagcgcgagcggttcaagaacgagggcctgggctcgctggtggcgctgaacgccttcctggcaacgcagccggcgaacgtgccgcgcgggcaggacgattacaccaactttatcagcgcgctgcggctgatggtgaccgaggtgccccagagcgaggtgtaccagtctggcccggactactttttccagacgagccggcagggcttgcagacggtgaacctgagccaggctttcaagaatctgcgcgggctgtggggcgtgcaggcgcccgtgggcgaccggtcaacggtgagcagcttgctgacgcccaactcgcggctgctgctgctgctgatcgcgcccttcaccgacagcggcagcgtgaaccgcaactcgtacctgggccatctgctgacgctgtaccgcgaggccataggccaggcgcaggtggacgagcagaccttccaggagatcactagcgtgagccgcgcgctggggcagaacgacaccgacagtctgagggccaccctgaactttttgctgaccaatagacagcagaagatcccggcgcagtacgcactgtcggccgaggaggaaaggattctgagatatgtgcagcagagcgtagggctgttcctgatgcaggagggtgccacccccagcgccgcgctggacatgaccgcgcgcaacatggaacctagcatgtacgccgccaaccggccgttcatcaataagctgatggactacttgcaccgcgcggcggccatgaacacggactactttaccaacgccatcctgaacccgcactggctcccgccgccggggttctacacgggcgagtacgacatgcccgaccccaacgacgggttcctgtgggacgacgtggacagcgcggtgttctcgccgacctttcaaaagcgccaggaggcgccgccgagcgagggcgcggtggggaggagcccctttcctagcttagggagtttgcatagcttgccgggctcggtgaacagcggcagggtgagccggccgcgcttgctgggcgaggacgagtacctgaacgactcgctgctgcagccgccgcgggccaagaacgccatggccaataacgggatagagagtctggtggacaaactgaaccgctggaagacctacgctcaggaccatagggacgcgcccgcgccgcggcgacagcgccacgaccggcagcggggcctggtgtgggacgacgaggactcggccgacgatagcagcgtgttggacttgggcgggagcggtggggtcaacccgttcgcgcatctgcagcccaaactggggcgacggatgttttgaatgaaataaaactcaccaaggccatagcgtgcgttctcttccttgttagagatgaggcgcgcggtggtgtcttcctctcctcctccctcgtacgagagcgtgatggcgcaggcgaccctggaggttccgtttgtgcctccgcggtatatggctcctacggagggcagaaacagcattcgttactcggagctggctccgcagtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacactaacatgcccaatgtgaacgagtacatgttcaccagcaagtttaaggcgcgggtgatggtgtctaggaagcatccagagggggtagttgaaacagatttgagtcaggataagcttgaatatgagtggtttgagtttaccctgcccgagggaaacttttccgagaccatgaccatagacctgatgaacaacgccatcttggaaaactacttgcaagtggggcggcagaatggcgtgctggagagcgatatcggagtcaagtttgacagcagaaatttcaagctgggctgggacccggtgaccaagctggtgatgccaggggtctacacctacgaggccttccacccggacgtggtgctgctgccgggctgcggggtggacttcaccgagagccgcctgagcaacctcctgggcattcgcaagaagcaacctttccaagagggcttcagaatcatgtatgaggatctagaaggtggcaacatccccgccctccttgatgtgcccaagtacttggaaagcaagaagaaagttgaagacgaaactaaaaatgcagctgcggccacagccgatacaaccactaggggtgatacatttgcaactccagcgcaagagacagcagctgataagaaggtagaagtcttgcccattgaaaaggatgagagtggtagaagttacaacctgatccaggggacccacgacacgctgtaccgcagttggtacctgtcctatacctacggggaccccgagaagggggtgcagtcgtggacgctgctcaccaccccggacgttacctgcggcgcggagcaagtctactggtcactgccggacctcatgcaagaccccgtcaccttccgctccacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgctaccgctgcgcagcagtatccgcggagtccagcgagtgaccgtcactgacgcccgtcgccgcacctgtccctacgtctacaaggccctgggcatagtcgcgccgcgcgtgctttccagtcgcaccttctaaaaaaatgtctattctcatctcgcccagcaataacaccggctggggtcttactagacccagcaccatgtacggaggagccaagaagcgctcccagcagcaccccgtccgcgtccgcggccacttccgcgctccctggggcgcttacaagcgcgggcggacttccaccgccgtgcgcaccaccgtcgacgacgtcatcgactcggtggtcgccgacgcgcgcaactacactcccgccccctccaccgtggacgcggtcatcgacagcgtggtggccgacgcgcgcgactatgccagacgcaagagccggcggcgacggatcgccaggcgccaccggagcacgcccgccatgcgcgccgcccgggctctgctgcgccgcgccagacgcacgggccgccgggccatgatgcgagccgcgcgccgcgctgccactgcacccacccccgcaggcaggactcgcagacgagcggccgccgccgccgctgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggaccaccccaggcggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggtcaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcaaaccgtggacattcagatccccaccgacatggatgtcgacaaaaaaccctcgaccagcatcgaggtgcaaaccgacccctggctcccagcctccaccgctaccgtctccacttctaccgccgccacggctaccgagcctcccaggaggcgaagatggggcgccgccagccggctgatgcccaactacgtgttgcatccttccatcatcccgacgccgggctaccgcggcacccggtactacgccagccgccggcgcccagccagcaaacgccgccgccgcaccgccacccgccgccgtctggcccccgcccgcgtgcgccgcgtgaccacgcgccggggccgctcgctcgttctgcccaccgtgcgctaccaccccagcatcctttaattcgtgtgctgtgatactgttgcagagagatggctctcacttgccgcctgcgcatccccgtcccgaattaccgaggaagatcccgccgcaggagaggcatggcaggcagcggcctgaaccgccgccggcggcgggccatgcgcaggcgcctgagtggcggctttctgcccgcgctcatccccataatcgccgcggccattggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccaaaacttccagcagaaggtggtggacgggctggcctcgggcattaacggggtggtggacatcgcgaaccaggccgtgcagcgcgagataaacagccgcctggacccgcggccgcccacggtggtggagatggaagatgcaactcttccgccgcccaaaggcgaaaagcggccgcggcccgacgcggaggagacgatcctgcaggtggacgagccgccctcgtacgaggaggccgtcaaggccggcatgcccaccacgcgcatcatcgcgccgctggccacgggtgtaatgaaacccgccacccttgacctgcctccaccacccgcgcccgctccaccgaaggcaactccggttgtgcaggcccccccggtggcgaccgccgtgcgccgcgtccccgcccgccgccaggcccagaactggcagagcacgctgcacagtatcgtgggcctgggagtgaaaagtctgaagcgccgccgatgctattgagagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccacgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacttactttgacatccgcggcgtcctggaccgcggtcccagcttcaaaccctactcgggcacggcctacaacagcctggctcccaagggtgcccccaatcccagtcagtgggaaacaaaagaaaagcaaggaactactggaggagtgcagcaagaaaaagatgtcacaaaaacatttggtgtggctgccaccggcggaattaatataacaaaccagggtctgttactaggaactgacgaaaccgctgagaatggcaaaaaagacatttatgcagacaagactttccagccagaacctcaagttggagaagaaaactggcaggaaaatgaagccttctatggaggaagggctcttaaaaaggacactaaaatgaaaccatgctatggatcttttgctagacctactaatgagaaaggaggtcaggcaaagttcaaaccagttaatgaaggagaacaacctaaagatctggatatagattttgcttactttgacgtccctggcggaagtcctccagcaggtggtagtggggaagaatacaaagcagatataattttgtacactgaaaatgttaatcttgaaacaccagacactcatgtggtttacaagccaggaacttcagataacagttcagaaatcaatctggttcagcagtccatgccaaacagacccaactacattggctttagggacaactttgtaggtctcatgtattacaacagcaccggaaatatgggtgtgctggctggtcaggcttctcagttgaacgctgtggtcgacttgcaagacagaaacaccgagttatcttaccagctattgctagattctctgggtgacagaaccagatactttagcatgtggaactctgcggtggacagttacgatccagatgtcaggatcattgaaaatcacggtgtggaagatgaacttccaaactattgcttcccattgaatggcactggaaccaattccacttatcaaggtgtaaagattacaaatggtaatgatggtgctgaagaaagtgagtgggagaaagacgatgcaatttctagacaaaaccaaatctgcaagggcaatgtctacgccatggagatcaacctgcaggccaacctgtggaagagttttctgtactcgaacgtggccctgtacctgcccgactcctacaagtacacgccggccaacgtcaagctgcccgccaacaccaacacctacgagtacatgaacggccgcgtggtagccccatccctggtggacgcctacatcaacatcggcgcccgctggtcgttggaccccatggacaacgtcaaccccttcaaccaccaccgcaatgcgggcctgcgctaccgctccatgctgctgggcaacggccgctacgtgcccttccacatccaagtgccccaaaagttctttgccatcaagaacctgctcctgctcccgggctcctacacctacgagtggaacttccgcaaggacgtcaacatgatcctgcagagttccctcggcaacgacctgcgcgtcgacggcgcctccgtccgcttcgacagcgtcaacctctacgccactttcttccccatggcgcacaacaccgcctccaccctggaagccatgctgcgcaacgacaccaacgaccagtccttcaacgactacctctcggccgccaacatgctctaccccatcccggccaaggccaccaacgtgcccatctccatcccatcgcgcaactgggccgccttccgcggctggagtttcacccggctcaagaccaaggaaactccttccctcggctcgggtttcgacccctactttgtctactcgggctccatcccctacctcgacgggaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgaccaaggactggttcctcgtccagatgctctcccactacaacatcggctaccagggcttccacgtgcccgagggctacaaggaccgcatgtactccttcttccgcaacttccagcccatgagcaggcaggtggtcgatgagatcaactacaaggactacaaggccgtcaccctgcccttccagcacaataactcgggcttcaccggctacctcgcacccaccatgcgccaggggcagccctaccccgccaacttcccctacccgctcatcggtcagacagccgtgccctccgtcacccagaaaaagttcctctgcgacagggtcatgtggcgcatcccattctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtacaccagccgcaccgcggcgtcatcgaggccgtctacctgcgcacgcccttctccgccggcaacgccaccacctaagcatgagcggctccagcgaacgagagctcgcggccatcgtgcgcgacctgggctgcgggccctactttttgggcacccacgacaagcgcttcccgggctttctcgccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgcagcgccctggcctcctcgcccgaccgctgtctcagcctcgagcagtccactcagaccgtgcaggggcccgactccgccgcctgcggactcttctgttgcatgttcttgcatgccttcgtgcactggcccgaccgacccatggacggaaaccccaccatgaacttgctgacgggggtgcccaacggcatgctacaatcgccacaggtgctgcccaccctcaggcgcaaccaggaggaactctaccgcttcctcgcgcgccactccccttactttcgctcccaccgcgccgccatcgaacacgccaccgcttttgacaaaatgaaacaactgcgtgtatctcaataaacagcacttttattttacatgcactggagtatatgcaagttatttaaaagtcgaaggggttctcgcgctcgtcgttgtgcgccgcgctggggagggccacgttgcggtactggtacttgggctgccacttgaactcggggatcaccagtttgggcactggggtctcggggaaggtctcgctccacatgcgccggctcatctgcagggcgcccagcatgtccggggcggagatcttgaaatcgcagttggggccggtgctctgcgcgcgcgagttgcggtacacggggttgcagcactggaacaccatcagactggggtacttcacactagccagcacgctcttgtcgctgatctgatccttgtccagatcctcggcgttgctcaggccgaacggggtcatcttgcacagctggcgtcccaggaagggcacgctctgaggcttgtggttacactcgcagtgcacgggcatcagcatcatccccgcgccgcgctgcatattcgggtagagggccttgacaaaggccgcgatctgcttgaaagcttgctgggccttggccccctcgctgaaaaacaggccgcagctcttcccgctgaactggttattcccacacccggcatcctgcacgcagcagcgcgcgtcatggctggtcagttgcaccacgctccgtccccagcggttctgggtcaccttagccttgctgggctgctccttcaacgcgcgctgcccgttctcgctggtcacatccatctccaccacgtggtccttgtggatcatcatcgtcccgtgcagacacttgagctggccttccacctcggtgcagccgtgatcccacagggcgcaaccggtgcactcccagttcttgtgcgcaatcccgctgtggctgaagatgtaaccttgcaacatgcggcccatgatggtgctaaatgctttctgggtggtgaaggtcagttgcatcccgcgggcctcctcgttcatccaggtctggcacatcttctggaagatctcggtctgctcgggcatgagcttgtaagcatcgcgcaggccgctgtcgacgcggtagcgttccatcagcacgttcatggtatccatgcccttctcccaggacgagaccagaggcagactcagagggttgcgtacgttcaggacaccgggggtcgcgggctcgacgatgcgttttccgtccttgccttccttcaatagaaccggcggctggctgaatcccactcccacgatcacggcatcttcctggggcatctcttcgtcggggtctaccttggtcacatgcttggtctttctggcttgcttcttttttggagggctgtccacggggagcacgtcctcctcggaagacccggagcccacccgctgatactttcggcgcttggtgggcagaggaggtggcggcgaggggctcctctcctgctccggcggatagcgcgccgacccgtggccccggggcggagtggcctctcggcccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctcgagcatggctacctgggaggagaggaggatgtgctgctgaaacacctgcagcgccagtccctcatcctccgggacgccctggccgaccggagcgaaacccccctcagcgtcgaggagctgtgtcgggcctacgagctcaacctcttctcgccgcgcgtaccccccaaacgccagcccaacggcacctgcgagcccaacccgcgtctcaacttctatcccgtctttgcggtccccgaagccctcgccacctatcacatctttttcaagaaccaaaagatccccgtctcctgccgcgccaaccgcaccagcgccgacgcgctcctcgctctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggccgtgctcaagcgcagcgtcgagctcacccacttcgcctaccccgccgtcaacctcccgcccaaggtcatgcgtcgcatcatggatcagctcatcatgccccacatcgaggccctcgatgaaagtcaggagcagcgccccgaggacgcccggcccgtggtcagcgacgagatgctcgcgcgctggctcgggacccacgacccccaggctttggaacagcggcgcaagctcatgctggccgtggtcctggttaccctcgagctggaatgcatgcgccgcttcttcagcgaccccgagaccctgcgcaaggtcgaggagaccctgcactacactttcagacacggtttcgtcaggcaggcctgcaagatctccaacgtggagctgaccaacctggtctcctgcctggggatcctgcacgagaaccgcctggggcagaccgtgctccactctaccctgaagggcgaggcgcggcgggactatgtccgcgactgcgtctttctatttctttgccacacatggcaagcagccatgggcgtgtggcaacagtgtctcgaggacgataacctgaaggagctggacaagcttcttgctagaaatcttaaaaagctgtggacgggcttcgacgagcgcaccgtcgcctcggacctggccgagatcgtgttccccgagcgcctgaggcagacgctgaaaggcgggctgcccgacttcatgagccagagcatgttgcaaaactaccgcactttcattctcgagcgatctgggatgctgcccgccacctgcaacgctttcccctccgactttgtcccgctgagctaccgcgagtgtcccccgccgctgtggagccactgctacctcttgcagctggccaactacatcgcctaccactcggacgtgatcgaggacgtgagcggcgaggggctgctcgagtgccactgccgctgcaacctgtgctccccgcaccgctccctggtctgcaacccccagctactaagcgagacccaggtcatcggtacctttgagctgcaaggtccgcaggagtccaccgctccgctgaaactcacgccggggttgtggacttccgcgtacctgcgcaaatttgtacccgaggactaccacgcccacgagataaagttcttcgaggaccaatcgcgtccgcagcacgcggatctcacggcctgcgtcatcacccagggcgcaatcctcgcccaattgcacgccatccaaaaatcccgccaagagtttcttctgaaaaagggtagaggggtctacctggacccccagacgggcgaagtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagaggagcaggcaacagagcagcccgtcgccgcaccatccgcgccggcagccccgccggtcacggatacaacctccgcagctccggccaagcctcctcgtagatgggatcgagtgaagggtgacggtaagcacgagcggcagggctaccgatcatggagggcccacaaagccgcgatcatcgcctgcttgcaagactgcggggggaacatcgctttcgcccgccgctacctgctcttccaccgcggggtaaacatcccccgcaacgtgttgcattactaccgtcaccttcacagctaagaaaaagcaagtaaaaggagtcgccggaggaggaggaggcctgaggatcgcggcgaacgagcccttgaccaccagggagctgaggaaccggatcttccccactctttatgccatttttcagcagagtcgaggtcagcagcaagagctcaaagtaaaaaaccggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccaaatgggcctggccgcgggcgcctcccaggactactccacccgcatgaactggctcagtgccggcccctcgatgatctcacgggtcaacggggtccgcagtcatcgaaaccagatattgttggagcaggcggcggtcacctccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctggtgatccgaggcagaggcacacagctcaacgacgagttggtgagctcttcgatcggtctgcgaccggacggagtgttccaactagccggagccgggagatcctccttcactcccaaccaggcctacctgaccttgcagagcagctcttcggagcctcgctccggaggcatcggaaccctccagtttgtggaggagtttgtgccctcggtctacttcaaccccttctcgggatcgccaggcctctacccggacgagttcataccgaacttcgacgcagtgagagaagcggtggacggctacgactgaatgtcccatggtgactcggctgagctcgctcggttgaggcatctggaccactgccgccgcctgcgctgcttcgcccgggagagctgcggactcatctactttgagtttcccgaggagcaccccaacggccctgcacacggagtgcggatcaccgtagagggcaccaccgagtctcacctggtcaggttcttcacccagcaacccttcctggtcgagcgggaccggggcgccaccacctacaccgtctactgcatctgtccaaccccgaagttgcatgagaatttttgttgtactctttgtggtgagtttaataaaagctaaactcttgcaatactctggaccttgtcgtcgtcaactcaacgagaccgtctacctcaccaaccagactgaggtaaaactcacctgcagaccacacaagacctatatcatctggttcttcgagaacacctcatttgcagtctccaacactcactgcaacgacggtgttgaacttcccaacaacctttccagtggactgagttacgatacacatagagctaagctcgtcctctacaatccttttgtagagggaacctaccagtgccagagcggaccttgtactcacaccttccatttggtgaacgtcaccagcagcagcaacagctcagaaactaaccttccttctgatactaacaaacctcgtttcggaggtgagctaaggcttcccccttctgaggagggggttagcccatacgaagtggtcgggtatttgattttaggggtggtcctgggtgggtgcatagcggtgctagctcagctgccttgctgggtggaaatcaaaatctttatatgctgggtcagatattgtggggaggaaccatgaaggggcttttgctgattatccttttcatggtggggggtgtactgtcatgccacgaacagccacgatgtaacatcaccacaggcaatcatatgagcagagagtgcactgtagtcatcaaatgcgagcacgactgcccactaaacattacattcaagaataacaccatgggaaatgtatgggtgggtttctgggaaccaggagatgagcagaactacacggtcactgtccatggtagcaatggaaatcacactttcggtttcaaattcatttttgaagtcatgtgtgatatcacactgcatgtggctagacttcatggcttgtggccccctaccaaggagaacatggttgggttttctttggcttttgtgatcatggcctgcttgatgtcaggtctgctggtaggggctttagtgtggttcctgaagcgcaagcctaggtacggaaatgaagaaaaggaaaaattgctataatctttttctttttcacagaaccatgaatgctttgaccagtgtcgtgctgctctctcttcttgtagcttttagtaatggggaagctgaaactgtagttgtaaatgttaaatctggtacaaaccacacccttgaaggtcctagaaaaactccagttcagtggtatgggggtgctaactttgacatgttttgcaatggctctaaaatacatcacaatgaattgaatcacacttgctctattcagaacataactcttacattcataaacagaacacatcatggaacatactatggttttggctctgacaatcaaaattcaaaagtgtatcatgtcagagtagatgtagagcctcctagaccccgtgctactttggctcctcctcaggacataactattaagtatggctcaaatagaacattgcagggcccaagtgttactccagttagttggtatgatggtgaaggaaatcggttttgcgatggcgataaaattgatcatacagaaattaatcacacttgcaatgctcaaaaccttactttgctgtttgtgaatgaaacacatgaaagaacatattatggaattagtggtgattggaaacagcgaaatgagtatgatgttactgttacaaagacacaattaaatattaaaaatttgggccaacgcaaaactgatgaaaaccataaaaatggaatgcatcagaaagtcgaacaaaatcctgaaactaagaaagaacagaagccttcaaaaagacctagacaaaaaacattgcaaactacaattcaggttatgattcctattggaactaattatactttagtggggccttcgccaccagtgagctggcatactacaaaaaatggcttaacagaactctgtaatggaaaccctattttaagacacacttgtgatgggcaaaatattacacttattaatgttaatgctacatttgaggctgattactatggctcgaacaataagagtgaatcaaaacactacagagtcaaggttttcaaagaaagaaaagatcaggcactattattcagaccgcttactaccaaaggaagcatgatcattactactgaaaatcaaaactttgaattacaacaaggtgacaatcaagatgatgacaaaattccatcaactactgtggcaatcgtggtgggtgtgattgcgggctttgtgactctgatcattgtcttcatatgctacatctgctgccgcaagcgtcccaggtcatacaatcatatggtagacccactactcagcttctcttactgaaactcagtcactctcatttcagaaccatgaaggctttcacagcttgcgttctgattagcatagtcacacttagtgcagctgaagctaaatgctttcatacttataacttaactagaggggaaaatattacattagcaggtgctggcttaaacacaacatgggaagcatatcacaatggatggaaacaagtttgtccatggaatgacggtcgctatgtgtgcgttggaaacagcagtaccataactaatcttacagttgtagctaatgcaaatttatcatcaactgttaaatttagagctgaaagtttatacattggaacagatggatatgaaagcaatccatcatgcttttatactatcaatgtaattgagcttccaaccaccagatcgccaactaccaccacggtcagtacaactactgagaccacaactcacactacacagttagacactacagtgcagaatagtactgtattggttaggtatttgttaagggaggaaagtactactgaacagacagaggctacctcaagcgccttcagcagcacttcaaatttaacttcgcttgcttggactaatgaaaccggagtatcattgatgcatggccagccttactcaggtttggatattcaaattacttttctggttgtctgtgggatctttattcttgtggttcttctgtactttgtctgctgcaaagccagagaaaaatctaggcggcccatctacaggccagtaatcggggaacctcagccactccaagtggatggaggcttaaggaatcttcttttctcttttacagtatggtgatcagccatgattcctaggttcttcctatttaacatcctcttctgtctcttcaacgtgtgcgctgccttcgcggccgtctcgcacgcctcacccgactgtctcgggcccttccccacctacctcctctttgccctgctcacctgcacctgcgtctgcagcattgtctgcctggtcatcaccttcctgcagctcatcgactggtgctgcgcgcgctacaattacctgcatcatagtcccgaatacagggacgagaacgtagccagaatcttaaggctcatatgaccatgcagactctgctcatactgctatccctcctatcccctaccctcgccacttctgctgattactctaaatgcaaattcgcggacatatggaatttcttagactgctatcaggagaaaattgacatgccctcctattacttggtgattgtgggaatagttatggtctgctcctgcactttctttgccatcatgatctacccctgttttgatctcggctggaactctgttgaagcattcacatacacactagaaagcagttcactagcctccacgccaccacccacaccgcctccccgcagaaatcagtttcccatgattcagtacttagaagagccccctccccgacccccttccactgttagctactttcacataaccggcggcgatgactgaccaccacctggacctcgagatggacggccaggcctccgagcagcgcatcctgcaactgcgcgtccgtcagcagcaggagcgtgccgccaaggagctcctcgatgccatcaacatccaccagtgcaagaagggcatcttctgcctggtcaaacaggcaaagatcacctacgagctcgtgtccggcggcaagcagcatcgcctcgcctatgagctgccccagcagaagcagaagttcacctgcatggtgggcgtcaaccccatagtcatcacccagcagtcgggcgagaccagcggctgcatccactgctcctgcgaaagccccgagtgcatctactccctgctcaagaccctttgcggactccgcgacctcctccccatgaactgatgttgattaaaagcccaaaaaccaatcagccccttcccccatttccccatcccccaattactcataaaaaataaatcattggaattaatcattcaataaagatcacttacttgaaatctgaaagtatgtctctggtgtagttgttcagcagcacctcggtaccctcctcccagctctggtactccagtccccggcgggcggcgaacttcctccacaccttgaaagggatgtcaaattcctggtccacaattttcattgtcttccctctcagatggcaaagaggctccgggtggaagatgacttcaaccccgtctacccctatggctacgcgcggaatcagaatatccccttcctcactcccccctttgtctcctccgatggattcaaaaacttcccccctggggtcctgtcacttaaactggctgatccaatcaccatcaacaatggggatgtctcacttaaggtgggagggggacttgctgtagagcaacagactggtaacctaagcgtaaaccctgatgcacccttgcaagttgcaagtgataagctacagcttgctctggctcctccattcgaggtcagagatggaaagcttgctttaaaggcaggtaatggattaaaagtactagataattccattactggattgactggattattgaatacacttgtggtattaactggaaggggaataggaacggaggaattaaaaaatgacgatggtgtaacaaacaaaggagtcggcttgcgtgtaagacttggagatgacggcgggctgacatttgataaaaagggtgatttagtagcctggaataaaaaagatgacaggcgcaccctgtggacaacccctgacacatctccaaattgcaaaatgagtacagaaaaggattctaaacttacgttgacacttacaaagtgtggaagtcaggttctgggaaatgtatctttacttgcagttacaggtgaatatcatcaaatgactgctactacaaagaaggatgtaaaaatatctttactatttgatgagaatggaattctattaccatcttcgtcccttagcaaagattattggaattacagaagtgatgattctattgtatctcaaaaatataataatgcagttccattcatgccaaacctgacagcttatccaaaaccaagcgctcaaaatgcaaaaaactattcaagaactaaaatcataagtaatgtctacttaggtgctcttacctaccaacctgtaattatcactattgcatttaatcaggaaactgaaaatggatgtgcttattctataacatttaccttcacttggcaaaaagactattctgcccaacagtttgatgttacatcttttaccttctcatatcttacccaagagaacaaagacaaagactaataaaatgttttgaactgaatttatgaatctttatttatttttacaccagcacgggtagtcagtttcccaccaccagcccatttcacagtgtaaacaattctctcagcacgggtggccttaaatagggaaatgttctgattagtgcgggaactggagtcgacctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatagctagccccgcttaccagtagacagagagcacagcagtacaagcgccaacagcagcgactgactacccactgacccagctccctatttaaaggcaccttacactgacgtaatgaccaaaggtctaaaaaccccgccaaaaaaacacacacgccctgggtgtttttcgcgaaaacacttccgcgttctcacttcctcgtatcgatttcgtgactcaacttccgggttcccacgttacgtcacttctgcccttacatgtaactcagccgtagggcgccatcttgcccacgtccaaaatggcttccatgtccggccacgcctccgcggcgaccgttagccgtgcgtcgtgacgtcatttgcatcaccgtttctcgtccaatcagcgttggctccgccccaaaaccgttaaaattcaaaagctcatttgcatattaacttttgtttactttgtggggtatatattgatgtttaaacaagcttgg

SEQ ID NO: 3 - DNA, 1521 bp, Artificial Sequence
  codon optimized HIV-1gag sequence
atgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagtaaaataaagcccgggcagat

SEQ ID NO: 4 - DNA, 2550 bp, Artificial Sequence
  gag expression cassette
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgagatctaccatgggtgctagggcttctgtgctgtctggtggtgagctggacaagtgggagaagatcaggctgaggcctggtggcaagaagaagtacaagctaaagcacattgtgtgggcctccagggagctggagaggtttgctgtgaaccctggcctgctggagacctctgaggggtgcaggcagatcctgggccagctccagccctccctgcaaacaggctctgaggagctgaggtccctgtacaacacagtggctaccctgtactgtgtgcaccagaagattgatgtgaaggacaccaaggaggccctggagaagattgaggaggagcagaacaagtccaagaagaaggcccagcaggctgctgctggcacaggcaactccagccaggtgtcccagaactaccccattgtgcagaacctccagggccagatggtgcaccaggccatctccccccggaccctgaatgcctgggtgaaggtggtggaggagaaggccttctcccctgaggtgatccccatgttctctgccctgtctgagggtgccaccccccaggacctgaacaccatgctgaacacagtggggggccatcaggctgccatgcagatgctgaaggagaccatcaatgaggaggctgctgagtgggacaggctgcatcctgtgcacgctggccccattgcccccggccagatgagggagcccaggggctctgacattgctggcaccacctccaccctccaggagcagattggctggatgaccaacaacccccccatccctgtgggggaaatctacaagaggtggatcatcctgggcctgaacaagattgtgaggatgtactcccccacctccatcctggacatcaggcagggccccaaggagcccttcagggactatgtggacaggttctacaagaccctgagggctgagcaggcctcccaggaggtgaagaactggatgacagagaccctgctggtgcagaatgccaaccctgactgcaagaccatcctgaaggccctgggccctgctgccaccctggaggagatgatgacagcctgccagggggtggggggccctggtcacaaggccagggtgctggctgaggccatgtcccaggtgaccaactccgccaccatcatgatgcagaggggcaacttcaggaaccagaggaagacagtgaagtgcttcaactgtggcaaggtgggccacattgccaagaactgtagggcccccaggaagaagggctgctggaagtgtggcaaggagggccaccagatgaaggactgcaatgagaggcaggccaacttcctgggcaaaatctggccctcccacaagggcaggcctggcaacttcctccagtccaggcctgagcccacagcccctcccgaggagtccttcaggtttggggaggagaagaccacccccagccagaagcaggagcccattgacaaggagctgtaccccctggcctccctgaggtccctgtttggcaacgacccctcctcccagtaaaataaagcccgggcagatctgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctcta

SEQ ID NO: 5 - DNA, 49 bp, Artificial Sequence
  short synthetic polyA signal
aataaaagatctttattttcattagatctgtgtgttggttttttgtgtg

SEQ ID NO: 6 - DNA, 2577 bp, Artificial Sequence
  codon optimized pol sequence with deletions in protease
agatctaccatggcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggatgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggatgacctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggatggggctgccaacagggagaccaagctgggcaaggctggctatgtgaccaacaggggcaggcagaaggtggtgaccctgactgacaccaccaaccagaagactgagctccaggccatctacctggccctccaggactctggcctggaggtgaacattgtgactgactcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggactgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgacaatggctccaacttcactggggccacagtgagggctgcctgctggtgggctggcatcaagcaggagtttggcatcccctacaacccccagtcccagggggtggtggagtccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 7 - protein, 850 aa, Artificial Sequence
  pol with altered protease
MAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 8 - DNA, 2577 bp, Artificial Sequence
  sequence for IA-pol
agatctaccatggcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggctgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggctgccctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggctggggctgccaacagggagaccaagctgggcaaggctggctatgtgaccaacaggggcaggcagaaggtggtgaccctgactgacaccaccaaccagaagactgccctccaggccatctacctggccctccaggactctggcctggaggtgaacattgtgactgcctcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggcctgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgccaatggctccaacttcactggggccacagtgagggctgcctgctggtgggctggcatcaagcaggagtttggcatcccctacaacccccagtcccagggggtggtggcctccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 9 - protein, 932 aa, Artificial Sequence
  IA-pol
MAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLTGCTGAGGTGGGGCCTGACCACCCCTGACAAGAAGCACCAGAAGGAGCCCCCCTTCCTGTGGATGGGCTATGAGCTGCACLRWGLTTPDKKHQKEPPFLWMGYELHPDKWTVQPIVLPEKDSWTVNDIQKLVGKLNWASQIYPGIKVRQLCKLLRGTKALETEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVAGAANRETKLGKAGYVTNRGRQKVVTLTDTTNQKTALQAIYLALQDSGLEVNIVTASQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLACTHLEGKVILVAVHVASGYIEAEVIPAETGQETAYFLLKLAGRWPVKTIHTANGSNFTGATVRAACWWAGIKQEFGIPYNPQSQGVVASMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDEDX

SEQ ID NO: 10 - DNA, 671 bp, Artificial Sequence
  codon optimized sequence for HIV-1 jrfl net
gatctgccaccatgggcggcaagtggtccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcctgctgcaccccatgtcccagcacggcatcgaggaccccgagaaggaggtgctggagtggaggttcgactccaagctggccttccaccacgtggccagggagctgcaccccgagtactacaaggactgctaaagcccgggc

SEQ ID NO: 11 - protein, 216 aa, Artificial Sequence
  HIV-1 jrfl nef
MGGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDC

SEQ ID NO: 12 - DNA, 651 bp, Artificial Sequence
  wild type nef sequence
atgggtggcaagtggtcaaaacgtagtgtgcctggatggtctactgtaagggaaagaatgagacgagctgagccagcagcagatagggtgagacgaactgagccagcagcagtaggggtgggagcagtatctcgagacctggaaaaacatggagcaatcacaagtagcaatacagcagctaccaatgctgattgtgcctggctagaagcacaagaggatgaggaagtgggttttccagtcagacctcaggtacctttaagaccaatgacttacaagggagctgtagatcttagccactttttaaaagaaaaggggggactggaagggctaattcactcacagaaaagacaagatatccttgatctgtgggtctaccacacacaaggctacttccctgattggcagaactacacaccagggccaggaatcagatttccattgacctttggatggtgcttcaagctagtaccagttgagccagaaaaggtagaagaggccaatgaaggagagaacaactgcttgttacaccctatgagccagcatgggatagaggacccggagaaggaagtgttagagtggaggtttgacagcaagctagcatttcatcacgtggcccgagagctgcatccggagtactacaaggactgctga

SEQ ID NO: 13 - DNA, 671 bp, Artificial Sequence
  sequence for opt nef (G2A, LLAA)
gatctgccaccatggccggcaagtggtccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcgccgcccaccccatgtcccagcacggcatcgaggaccccgagaaggaggtgctggagtggaggttcgactccaagctggccttccaccacgtggccagggagctgcaccccgagtactacaaggactgctaaagcccgggc

SEQ ID NO: 14 - protein, 216 aa, Artificial Sequence
  opt nef (G2A, LLAA)
MAGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCAAHPMSQHGIEDPEKEVLEWRFDSKLAFHHVARELHPEYYKDC

SEQ ID NO: 15 - DNA, 671 bp, Artificial Sequence
  sequence for opt nef (G2A)
gatctgccaccatggccggcaagtggtccaagaggtccgtgcccggctggtccaccgtgagggagaggatgaggagggccgagcccgccgccgacagggtgaggaggaccgagcccgccgcagtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcctgctgcaccccatgtcccagcacggcatcgaggaccccgagaaggaggtgctggagtggaggttcgactccaagctggccttccaccacgtggccagggagctgcaccccgagtactacaaggactgctaaagcccgggc

SEQ ID NO: 16 - protein, 216 aa, Artificial Sequence
  opt nef (G2A)
MAGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDC

SEQ ID NO: 17 - DNA, 2662 bp, Artificial Sequence
  SEAP expression cassette
ccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgagatctaagtaagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtccccgcgttgcttcctctgctggccgggacatcaggtggcccccgctgaattggaatcgatcagaattcagtcgacgatatctgatcacgatctgatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctcta